(19) United States
(12) Patent Application Publication
(10) Pub. No.: US 2022/0257781 A1
(43) Pub. Date: Aug. 18, 2022

(54) POLYMER CONJUGATE BONDED TO TERTIARY AMINE COMPOUND OR IMINE COMPOUND AND PRODUCTION METHOD THEREFOR

(71) Applicant: Seikagaku Corporation, Tokyo (JP)

(72) Inventors: Nobuo Kobayashi, Tokyo (JP); Kenichi Namatsu, Tokyo (JP)

(73) Assignee: Seikagaku Corporation, Tokyo (JP)

(21) Appl. No.: 17/274,073

(22) PCT Filed: Sep. 5, 2019
(86) PCT No.: PCT/JP2019/035074

§ 371 Date: Mar. 5, 2021

(30) Foreign Application Priority Data
Sep. 6, 2018 (JP) 2018-166999

(51) Int. Cl. A61K 47/59; A61K 47/61

(52) U.S. Cl. CPC A61K 47/595; A61K 47/61

(57) ABSTRACT

Provided is a compound as a compound obtained by conjugating a tertiary amine compound or an imine compound, which is useful as a drug, with a polymer, in which a structure D⁺ forming a quaternary ammonium salt or an iminium salt from D, a tertiary amine compound or an imine compound and a polymer residue Poly having a carboxy group are bonded to each other via a structure —C(R¹)(R²)OC(═O)YANHC(═O)—.

### TECHNICAL FIELD

The present invention relates to a novel conjugate of a tertiary amine compound or an imine compound with a polymer and a production method therefor. Specifically, the present invention relates to a novel conjugate of a tertiary amine compound or an imine compound with a polymer using, as a linker, an aminoalkoxy carbonyloxymethyl group and (aminoalkyl) aminocarbonyloxymethyl group whose release rate can be controlled.

### BACKGROUND ART

A conjugate of a drug with a polymer has been widely reviewed in a field of a prodrug or drug delivery system (DDS), and is an important means for providing a function such as release control, absorption improvement, stabilization in a living body, or targeting to a target tissue.

For example, a conjugate of polyglutamic acid that is one of polyamine acids with a therapeutic drug has been reported in JP 2003-511423 A. A conjugate of sodium carboxymethyl cellulose that is used as a Pharmaceutical additive with gossypol has been reported in JP 5690944 B2. Alginic acid that is one of dietary fibers in polysaccharides has also been reviewed as a polymer used for the conjugate, and conjugates of alginic acid with various drugs have been reported in JP H08-24325 A. Further, glycosaminoglycan has also been widely reviewed as a polymer used in a conjugate, and a conjugate of hyaluronic acid or chondroitin sulfate with a peptide has been reported in U.S. Pat. No. 5,955,578. In addition, conjugates of heparin with various drugs have been reported in WO 93/18793 A. Further, hyaluronic acid has also been reviewed as a polymer used in a conjugate, a conjugate of hyaluronic acid with taxane has been reported in WO 2005/085294 A, and a conjugate of hyaluronic acid with a protein such as a serine protease inhibitor has been reported in JP 2006-504747 A.

Meanwhile, a method of conjugating a polymer with a drug is roughly classified into two types: 1) a method of directly bonding a polymer to a drug (JP 2006-504747 A and the like) and 2) a method of bonding a polymer to a drug via a linker (JP 2003-511423 A and the like).

When a structure of a drug to be bonded to and conjugated with a polymer is confirmed, a drug having an amino group, a carboxy group, or a hydroxyl group as a functional group in the molecule is utilized. In a bonding mode thereof, regarding a drug having a primary or secondary amino group, a method of bonding by reductive amination with a drug having a primary amino group (JP 2000-501082 A) and a method of forming an amide bond with a drug having a primary or secondary amino group (JP H08-24325 A) have been known.

### CITATION LIST

### Patent Literature

Patent Literature 1: JP 2003-511423 A

Patent Literature 2: JP 5690944 B2

Patent Literature 3: JP H08-24325 A

Patent Literature 4: U.S. Pat. No. 5,955,578

Patent Literature 5: WO 1993/18793 A

Patent Literature 6: WO 2005/085294 A.

Patent Literature 7: JP 2006-504747 A

Patent Literature 8: JP 2000-501082 A

### SUMMARY OF INVENTION

In a method of related art, a current situation is that it cannot be said that release control of a tertiary amine compound or an imine compound conjugated with a polymer is sufficiently achieved. Since conjugation reaction is selected depending on a functional group of a drug, a conjugate with the tertiary amine compound or the imine compound cannot be obtained by a method of the related art, and thus construction of a novel method has been desired.

An object of the present invention is to provide a novel conjugate of a tertiary amine compound or an imine compound with a polymer having a carboxy group, the conjugate whose release rate can be controlled, and a production method therefor.

The present inventors have conducted intensive studies on a linker capable of forming a conjugate of a tertiary amine compound or an imine compound with a polymer having a carboxy group, and as a result, have found aminoalkoxy carbonyloxymethyl group and (aminoalkyl) aminocarbonyloxyl nethyl group linkers whose release rate can be controlled. The present invention is based on the finding of a linker which has not existed hitherto and by which a tertiary amine compound or an imine compound can be bonded to a polymer having a carboxy group in the form in which a release rate can be controlled, and relates to a novel tertiary amine compound or imine compound-polymer conjugate and a production method therefor.

### BRIEF DESCRIPTION OF DRAWINGS

FIG. 1 is a graph showing a relation between time and a drug release ratio in a buffer solution having a pH of 7.0 in Examples 5, 13, 1, 16, and 2.

FIG. 2 is a graph showing a relation between time and a drug release ratio in a buffer solution having a pH of 7.0 in Examples 6 and 3.

FIG. 3 is a graph showing a relation between time and a drug release ratio in a buffer solution having a pH of 7.0 in Examples 11, 7, 10, 8, and 20.

FIG. 4 is a graph showing a relation between time and a drug release ratio in a buffer solution having a pH of 7.0 in Examples 15 and 17.

FIG. 5 is a graph showing a relation between time and a drug release ratio in a buffer solution having a pH of 7.0 in Examples 14 and 18.

FIG. 6 is a graph showing a relation between time and a drug release ratio in a buffer solution having a pH of 7.0 in Example 19.

FIG. 7 is a graph showing a relation between time and a drug release ratio in a buffer solution having a pH of 7.0 in Examples 21, 22, 23, and 26.

FIG. 8 is a graph showing a relation between time and a drug release ratio in a buffer solution having a pH of 7.0 in Examples 25 and 27.

### DESCRIPTION OF EMBODIMENTS

A conjugate (hereinafter, also referred to as “a tertiary amine compound or imine compound-polymer conjugate” in some cases) according to an aspect of the present invention is a compound having a structure represented by the following Formula (I) or a pharmaceutically acceptable salt thereof;

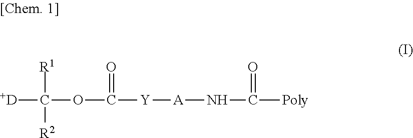

in Formula (I), D⁺ is a structure forming a quaternary ammonium salt or an iminium salt from a tertiary amine compound or an imine compound D; a nitrogen atom of D⁺ forming the quaternary ammonium salt or the iminium salt and a carbon atom to which R¹ and R² bond are bonded to each other; Y is O or NR³; R¹, R², and R³ are each independently a hydrogen atom, a substituted or unsubstituted alkyl group, a substituted or unsubstituted cycloalkyl group, a substituted or unsubstituted alkenyl group, a substituted or unsubstituted cycloalkenyl group, a substituted or unsubstituted alkynyl group, a substituted or unsubstituted aromatic group, or a substituted or unsubstituted heterocyclic group; A is a substituted or unsubstituted bivalent hydrocarbon group, may contain one or more heteroatoms at a position except for both ends which are bonded to —Y— or —NH—, and the heteroatoms are each independently selected from the group consisting of —O—, —NH— which may have a substituent, and —S—; any two or three groups of R¹, R², R³, and A may combine together to form a ring; and Poly is a polymer residue and —C(═O)— adjacent to Poly is derived from a carboxy group of the polymer.

A structure represented by Poly which is derived from a polymer having a carboxy group and a structure represented by D⁺ having a quaternary ammonium salt or iminium salt formed from a tertiary amine compound or an imine compound D are bonded to each other via a linker including Y and A to form a conjugate. The conjugate is preferably a conjugate in which a drug containing a tertiary amine or imine structure is D.

In a bioactive substance including a medical drug, extremely many tertiary amine compounds or imine compounds exist, but in the techniques of the related art, there is no means for bonding those compounds to a polymer having a carboxy group in the form in which a release rate can be controlled. The linker having the structure found in the present invention enables a conjugate of a tertiary amine compound or imine compound with a polymer having a carboxy group which cannot be prepared hitherto to be produced. Further, the technique according to the present invention provides a new function (for example, sustained-release performance or improved retention in blood or administered tissue) by conjugating various bioactive substances and considerably contributes to medical treatment and the like.

The conjugate bonds to a hydrocarbon chain (bivalent hydrocarbon group represented by A in the above Formula (I)) of the linker when the carboxy group of the polymer residue forms an amide bond.

In the present invention, by setting an amino group at the terminal of the hydrocarbon chain of the linker to be bonded to the polymer, it is possible to form a bond (amide bond) with a polymer even in a protic solvent such as water, and particularly, in a reaction in which a bonded form (amine form represented by Formula (III) or (V) described later) of the tertiary amine compound or imine compound with the linker is bonded with the polymer to complete the conjugate represented by the above Formula (I) or Formula (II) described later, it is possible to perform the reaction in a protic solvent such as water. That is, by setting an amino group at the terminal of the linker, even in a reaction in an environment where multiple and various functional groups coexist (a reaction in a protic solvent such as water or a reaction with a polymer having multiple types of functional groups), the polymer having a carboxy group and the amino group of the linker can be site-selectively amide-bonded. This leads to a decrease in waste liquid of an aprotic solvent, and the like.

The bivalent hydrocarbon group represented by A may be a carbon chain having carbon number of not less than 2 and may have a branched structure or a cyclic structure. Further, the bivalent hydrocarbon group represented by A may contain one or more heteroatoms at a position except for both ends which are bonded to —Y— or —NH—, and the heteroatoms are each independently selected from the group consisting of —O—, —NH— which may have a substituent and —S—. Furthermore, any two or three groups of R¹, R², R³, and A can also combine together to form a ring.

The conjugate represented by the above Formula (I) is preferably a compound represented by the following Formula (H) or a pharmaceutically acceptable salt thereof:

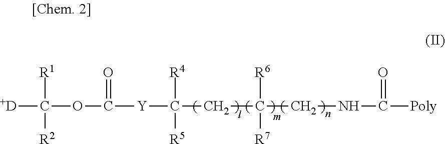

in Formula (H), D⁺, R¹, R², Y, and Poly are as defined above; R⁴, R⁵, R⁶, and R⁷ are each independently a hydrogen atom, a substituted or unsubstituted alkyl group, a substituted or unsubstituted cycloalkyl group, a substituted or substituted alkenyl group, a substituted or unsubstituted cycloalkenyl group, a substituted or unsubstituted alkynyl group, a substituted or unsubstituted aromatic group, or a substituted or substituted heterocyclic group; any two or three groups of R¹, R², R³, R⁴, R⁵, R⁶, and R⁷ may be combine together to form a ring; 1 and n are each independently are 0, 1, or 2; and m is 0 or 1.

Therefore, A is preferably a bivalent hydrocarbon group represented by C(R⁴)(R⁵)—(CH₂)ₗ—(C(R⁶)(R⁷))ₘ—(CH₂)ₙ as represented by the above Formula (II) (herein, R⁴, R⁵, R⁶, R⁷, l, m, and n are as defined above). From the viewpoint of ease of design and availability of raw materials, A is preferably a linear or branched alkylene group having carbon number of 2 to 10, and the carbon number of A is further preferably 2 to 6. Further, from the viewpoint of controlling the release rate, A in the above Formula (I) may have a branched chain or a substituent particularly in the carbon adjacent to Y. For example, at least one of R³, R⁴, R⁵, R⁶, and R⁷, particularly, at least one of R⁴ and R⁵ in the above Formula (II) may be a group other than hydrogen,

In the above Formula (I), A is bonded to a substituted or unsubstituted methylene group represented by —C(R¹)(R²)— via a carbonic ester bond or urethane bond represented by —O—C(═O)—Y—. In Formula (I), an oxygen atom of a carbonic ester bond or urethane bond-the methylene group-a nitrogen atom forming a quaternary ammonium salt or iminium salt are bonded in this order. The methylene group may be bonded to the bivalent hydrocarbon group to form a ring. The tertiary amine compound or imine compound exists in the structure of the conjugate as the quaternary ammonium salt or iminium salt by being bonded to a structure derived from a polymer having a carboxy group via the linker.

D can rapidly release the tertiary amine compound or imine compound D owing to the presence of an oxymethylene group to be bonded to D⁺. This mechanism will be described using the compound represented by Formula (I) as follows. Regarding the tertiary amine compound or imine compound-polymer conjugate represented by the above Formula (I), a carbonic ester bond moiety or urethane bond moiety is hydrolyzed in the presence of water to be decomposed into a hydroxymethyl form represented by Formula (VIII) and a carbonic monoester form or carbamic acid form represented by Formula (IX). Since the carbonic monoester form or carbamic acid form represented by Formula (IX) is unstable in terms of the structure, the carbonic monoester form or carbamic acid form is rapidly decomposed into an alcohol form or amine form represented by Formula (XI) and carbon dioxide. Furthermore, the hydroxymethyl form represented by Formula (VIII) is unstable in terms of the structure since the hydroxymethyl form has a quaternary ammonium or iminium structure, and thus the hydroxymethyl form is rapidly decomposed into the tertiary amine compound or imine compound D and an aldehyde form (or a ketone form) represented by Formula (X). At this time, the function of the generated tertiary amine compound or imine compound is exhibited, Therefore, the tertiary amine compound or imine compound-polymer conjugate represented by the above Formula (I) can control releasing of the tertiary amine compound or imine compound by controlling a hydrolysis rate of the carbonic ester bond moiety or urethane bond moiety, so that sustainability of the function of the tertiary amine compound or imine compound can be controlled. Further, as clearly shown from Examples described later, from the viewpoint that the release rate of the tertiary amine compound or imine compound can be reduced, in the case of selecting the tertiary amine compound or imine compound-polymer conjugate represented by the above Formula (I) or (II), it is preferable to have a urethane bond moiety represented by —O—C(═O)—NR³— (that is, the above Formula (I) or (II) in which Y is NR³).

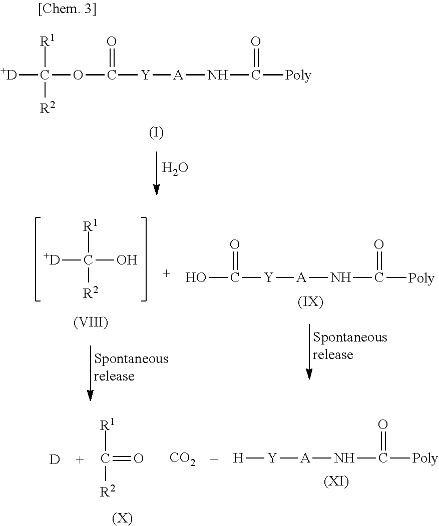

One embodiment of the tertiary amine compound or imine compound-polymer conjugate in the present invention is the compound represented by the above Formula (I) or (II), and the amine form that is an important intermediate of the compound structure represented by (I) or (II) is a compound represented by the following Formula (III) or (V).

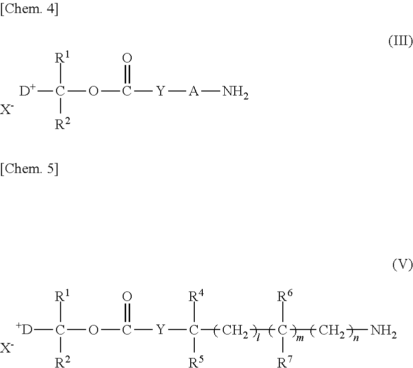

in Formula (III) or (V), D⁺, R¹, R², R⁴, R⁶, R⁷, Y, A, l, m, and n are as defined above; and X⁻ is a counter anion of the quaternary ammonium salt or the iminium salt in D⁺. The compound represented by the above Formula (III) or (V) may further form a salt with an inorganic acid or an organic acid.

Specific examples of the alkyl group, the cycloalkyl group, the alkenyl group, the cycloalkenyl group, the alkynyl group, the aromatic group, and the heterocyclic group included in the groups represented by the substituents R¹, R², R⁴, R⁵, R⁶, and R⁷ in Formulae (I), (II), (III), and (V) include the following groups.

As the alkyl group, any of a linear or branched chain alkyl group may be used. Further, the number of carbon atoms of the alkyl group is preferably 1, 2, 3, 4, 5, or 6. Examples of the alkyl group may include a methyl group, an ethyl group, an n-propyl group, a 2-propyl, a n-butyl group, a 1-methylpropyl group, a 1,1-dimethylethyl group, a 2-methylpropyl group, an n-pentyl group, a 1-methylbutyl group, a 2-methylbutyl group, a 3-methylbutyl group, a 1-ethylpropyl group, a 1,1-dimethylpropyl group, a 12-dimethylpropyl group, a 2,2-dhnethylpropyl group, a n-hexyl group, a 1-methylpentyl group, a 2-methylpentyl group, a 3-methylpentyl group, a 4-methylpentyl group, a 1-ethylbutyl group, a 2-ethylbutyl group, a 1,1-dimethylbutyl group, a 1,2-dimethylbutyl group, a 1,3-dimethylbutyl group, a 2,2-dimethylbutyl group, a 2,3-dimethylbutyl group, a 3,3-dimethylbutyl group, a 1,1,2-trimethylpropyl group, a 1-ethyl-1-methylpropyl group, and a 1-ethyl-2-methylpropyl group.

Any cycloalkyl group may be used as long as the carbon atom at the node is included as an atom constituting a ring, and the cycloalkyl group may be condensed with cycloalkane, cycloalkene an aromatic ring, or a hetero ring and may form a spiro ring. Further, the number of carbon atoms of the cycloalkyl group is preferably 3, 4, 5, 6, 7, or 8. Examples of the cycloalkyl group may include a cyclopropyl group, a cyclobutyl group, a cyclopentyl group, a cyclohexyl group, a cycloheptyl group, and a cyclooctyl group.

As the alkenyl group, any of a linear, branched, or cyclic alkenyl group may be used. Further, the number of carbon atoms of the alkenyl group is preferably 2, 3, 4, 5, or 6. Examples of the alkenyl group may include a vinyl group, a 1-propenyl group, a 2-propenyl group, a 1-methylvinyl group, a 1-butenyl group, a 2-butenyl group, a 3-butenyl group, a 1-ethylvinyl group, a 1-methyl-1-propenyl group, a 1-methyl-2-propenyl group, a 2-methyl-1-propenyl group, a 2-methyl-2-propenyl group, a 1-pentenyl group, a 2-pentenyl group, a 3-pentenyl group, a 4-pentenyl group, a 1-propylvinyl group, a 1-methyl-1-butenyl group, a 1-methyl-2-butenyl group, a 1-methyl-3-butenyl group, a 2-methyl-1-butenyl group, a 2-methyl-2-butenyl group, a 2-methyl-3-butenyl group, a 3-methyl-1-butenyl group, a 3-methyl-2-butenyl group, a 3-methyl-3-butenyl group, a 1-ethyl-1-propenyl group, a 1-ethyl-2-propenyl group, a 1-(2-methylethyl)vinyl group, a 1,2-dimethyl-1-propenyl group, a 1,2-dimethyl-2-propenyl group, a 1,1-dimethyl-2-propenyl group, a 1-hexenyl group, a 2-hexenyl group, a 3-hexenyl group, a 4-hexenyl group, a 5-hexenyl group, a 1-butylvinyl group, a 1-methyl-1-pentenyl group, a 1-methyl-2-pentenyl group, a 1-methyl-3-pentenyl group, a 1-methyl-4-pentenyl group, a 2-methyl-1-pentenyl group, a 2-methyl-2-pentenyl group, a 2-methyl-3-pentenyl group, a 2-methyl-4-pentenyl group, a 3-methyl-1-pentenyl group, a 3-methyl-2-pentenyl group, a 3-methyl-3-pentenyl group, a 3-methyl-4-pentenyl group, a 4-methyl-1-pentenyl group, a 4-methyl-2-pentenyl group, a 4-methyl-3-pentenyl group, a 4-methyl-4-pentenyl group, a 1-propyl-1-propenyl group, a 1-propyl-2-propenyl group, a 1-ethyl-1-butenyl group, a 1-ethyl-2-butenyl group, a 1-ethyl-3-butenyl group, a 2-ethyl-1-butenyl group, a 2-ethyl-2-butenyl group, a 2-ethyl-3-butenyl group, a 1-(2-methylpropyl)vinyl group, a 1,2-dimethyl-1-butenyl group, a 1,2-dimethyl-2-butenyl group, a 1,2-dimethyl-3-butenyl group, a 1-(3-methylpropyl)vinyl group, a 1,3-dimethyl-1-butenyl group, a 1,3-dimethyl-2-butenyl group, a 1,3-dimethyl-3-butenyl group, a 2,3-dimethyl-1-butenyl group, a 2,3-dimethyl-2-butenyl group, a 2,3-dimethyl-3-butenyl group, a 3,3-dimethyl-1-butenyl group, a 2,2-dimethyl-3-butenyl group, a 1,1-dimethyl-2-butenyl group, a 1,1-dimethyl-3-butenyl group, a 1,1,2-dimethyl-2-propenyl group, a 1-ethyl-1-methyl-2-propenyl group, a 1-ethyl-2-methyl-1-propenyl group, a 1-ethyl-2-methyl-2-propenyl group, a 1-(1-methylethyl)-1-propenyl group, and a 1-(1-methylethyl)-2-propenyl group.

Any cycloalkenyl group may be used as long as the carbon atom at the node and a C═C double bond are included as an atom constituting a ring, and the cycloalkenyl group may be condensed with cycloalkane, cycloalkene, an aromatic ring, or a hetero ring and may form a Spiro ring. Further, the number of carbon atoms of the cycloalkenyl group is preferably 3, 4, 5, 6, 7, or 8. Examples of the cycloalkenyl group may include a 1-cyclopropen-1-yl group, a 2-cyclopropen-1-yl group, a 1-cyclobuten-1-yl group, a 2-cyclobuten-1-yl group, a 1-cyclopenten-1-yl group, a 2-cyclopenten-1-yl group, a 3-cyclopenten-1-yl group, a 1-cyclohexen-1-yl group, a 2-cyclohexen-1-yl group, a 3-cyclohexen-1-yl group, a 1-cyclohepten-1-yl group, a 2-cyclohepten-1-yl group, a 3-cyclohepten-1-yl group, a 4-cyclohepten-1-yl group, a 1-cycloocten-1-yl group, a 2-cycloocten-1-yl group, a 3-cycloocten-1-yl group, a 4-cycloocten-1-yl group, a 1,3-cyclopentadien-1-yl group, a 2,4-cyclopentadien-1-yl group, a 1,3-cyclohexadien-1-yl group, a 1,4-cyclohexadien-1-yl group, a 1,5-cyclohexadien-1-yl group, a 2,4-cyclohexadien-1-yl group, a 2,5-cyclohexadien-1-yl group, a 1,3-cycloheptadien-1-yl group, a 1,4-cycloheptadien-1-yl group, a 1,5-cycloheptadien-1-yl group, a 1,6-cycloheptadien-1-yl group, a 2,4-cycloheptadien-1-yl group, a 2,5-cycloheptadien-1-yl group, a 2,6-cycloheptadien-1-yl group, a 1,4-cycloheptadien-1-yl group, a 1,5-cycloheptadien-1-yl group, a 3,5-cycloheptadien-1-yl group, a 1,3-cyclooctadien-1-yl group, a 1,4-cyclooctadien-1-yl group, a 1,5-cyclooctadien-1-yl group, a 1,6-cyclooctadien-1-yl group, a 1,7-cyclooctadien-1-yl group, a 2,4-cyclooctadien-1-yl group, a 2,5-cyclooctadien-1-yl group, a 2,6-cyclooctadien-1-yl group, a 2,7-cyclooctadien-1-yl group, a 3,5-cyclooctadien-1-yl group, a 3,6-cyclooctadien-1-yl group, a 1,3,5-cycloheptatrien-1-yl group, a 1,3,6-cycloheptatrien-1-yl group, a 1,4,6-cycloheptatrien-1-yl group, a 2,4,6-cycloheptatrien-1-yl group, a 1,3,5-cyclooctatrien-1-yl group, a 1,3,6-cyclooctatrien-1-yl group, a 1,3,7-cyclooctatrien-1-yl group, a 1,4,6-cyclooctatrien-1-yl group, a 1,4,7-cyclooctatrien-1-yl group, a 1,5,7-cyclooctatrien-1-yl group, a 2,4,6-cyclooctatrien-1-yl group, a 2,4,7-cyclooctatrien-1-yl group, and a cyclooctatetraen-1-yl group.

As the alkynyl group, any of a linear, branched chain, or cyclic alkynyl group may be used. Further, the number of carbon atoms of the alkynyl group is preferably 2, 3, 4, 5, or 6. Examples of the alkynyl group may include an ethynyl group, a 1-propynyl group, a 2-propynyl group, a 1-butynyl group, a 2-butynyl group, a 3-butynyl group, a 1-methyl-2-propynyl group, a 1-pentynyl group, a 2-pentynyl group, a 3-pentynyl group, a 4-pentynyl group, a 1-methyl-2-butyryl group, a 1-methyl-3-butynyl group, a 2-methyl-3-butynyl group, a 3-methyl-1-butyryl group, a 1-ethyl-2-propynyl group, a 1,1-dimethyl-2-propynyl group, a 1-hexynyl group, a 2-hexynyl group, a 3-hexynyl group, a 4-hexynyl group, a 1-methyl-2-pentynyl group, a 1-methyl-3-pentynyl group, a 1-methyl-4-pentynyl group, a 2-methyl-3-pentynyl group, a 2-methyl-4-pentynyl group, a 3-methyl-1-pentynyl group, a 3-methyl-4-pentynyl group, a 4-methyl-1-pentynyl group, a 4-methyl-2-pentynyl group, a 1-butyl-2-propynyl group, a 1-ethyl-2-butyryl group, a 1-ethyl-3-butyryl group, a 2-ethyl-3-butyryl group, a 1,1-dimethyl-2-butyryl group, a 1,1-dimethyl-3-butynyl group, a 1,2-dimethyl-3-butyryl group, a 2,2-dimethyl-3-butynyl group, a 3,3-dimethyl-1-butynyl group, a 1-ethyl-1-methyl-2-propynyl group, a 1-(2-methylethyl)-2-propynyl group, a 2-cyclohexin-1-yl group, and a 3-cyclohexin-1-yl group.

As the aromatic group, a monocyclic or polycyclic aromatic group may be used, and the aromatic group may be condensed with a cycloalkane, a cycloalkene, an aromatic ring, or a hetero ring. Further, the number of carbon atoms of the aromatic group is preferably 6, 7, 8, 9, 10, 11, 12, 13, or 14. Examples of the aromatic group may include a phenyl group, a naphthyl group, and an anthracenyl group.

The heterocyclic group contains at least one or more of heteroatoms such as a nitrogen atom, an oxygen atom, or a sulfur atom as a ring-constituting atom, and those atoms may be condensed with a cycloalkane, a cycloalkene, an aromatic ring, or a hetero ring or form a Spiro ring. Further, the size of the ring of the heterocyclic group is preferably a 3-, 4-, 5-, 6-, 7- or 8-membered ring. Examples of the heterocyclic group may include an aziridinyl group, an azetidinyl group, a diazetidinyl group, a pyrrolidinyl group, a piperidino group, a homopiperidino group, a pyrazolidinyl group, an imidazolidinyl group, a triazolidinyl group, a tetrazolidinyl group, an oxazolidinyl group, an isoxazolidinyl group, a thiazolidinyl group, an isothiazolidinyl group, an oxadiazolidinyl group, a thiadiazolidinyl group, a piperazinyl group, a homopiperazinyl group, a triazepanyl group, a morpholino group, a thiomorpholino group, a quinuclidinyl group, a tropanyl group, a pyrrolinyl group, a pyrazolinyl group, an imidazolinyl group, an oxazolinyl group, a thiazolinyl group, an isooxazolinyl group, an isothiazolinyl group, a pyrrolyl group, an imidazolyl group, a pyrazolyl group, an oxazolyl group, a dihydrooxazolyl group, a tetrahydrooxazolyl group, an isooxazolyl group, a dihydroisooxazolyl group, a tetrahydroisooxazolyl group, a thiazolyl group, a dihydrothiazolyl group, a tetrahydrothiazolyl group, an isothiazolyl group, a dihydroisothiazolyl group, a tetrahydroisothiazolyl group, a triazolinyl group, a triazolyl group, an oxodiazolyl group, a dihydrooxodiazolyl group, a tetrahydrooxodiazolyl group, a thiadiazolyl group, a dihydrothiadiazolyl group, a tetrahydrothiadiazolyl group, a tetrazolinyl group, a tetrazolyl group, a furazanyl group, a dihydrofurazanyl group, a tetrahydrofurazanyl group, a piperideinyl group, a triazinanyl group, a pyridyl group, a dihydropyridyl group, a tetrahydropyridyl group, a pyrazinyl group, a dihydropyrazinyl group, a tetrahydropyrazinyl group, a pyrimidinyl group, a dihydropyrimidinyl group, a tetrahydropyrimidinyl group, a perhydropyrimidinyl group, a pyridazinyl group, a dihydropyridazinyl group, a tetrahydropyridazinyl group, a perhydropyridazinyl group, a triazinyl group, a dihydrotriazinyl group, a tetrahydrotriazinyl group, an oxazinyl group, a dihydrooxazinyl group, a tetrahydrooxazinyl group, an oxadiazinyl group, a dihydrooxadiazinyl group, a tetrahydrooxadiazinyl group, a thiazinyl group, a dihydrothiazinyl group, a tetrahydrothiazinyl group, a thiadiazinyl group, a dihydrothiadiazinyl group, a tetrahydrothiadiazinyl group, an azepinyl group, a dihydroazepinyl group, a tetrahydroazepinyl group, a perhydroazepinyl group, a diazepinyl group, a dihydrodiazepinyl group, a tetrahydrodiazepinyl group, a perhydrodiazepinyl group, an oxazepinyl group, a dihydrooxazepinyl group, a tetrahydrooxazepinyl group, a perhydrooxazepinyl group, an oxadiazepinyl group, a dihydrooxadiazepinyl group, a tetrahydrooxadiazepinyl group, a perhydrooxadiazepinyl group, a thiazepinyl group, a dihydrothiazepinyl group, a tetrahydrothiazepinyl group, a perhydrothiazepinyl group, a thiadiazepinyl group, a dihydrothiadiazepinyl group, a tetrahydrothiadiazepinyl group, a perhydrothiadiazepinyl group, a triazepinyl group, a dihydrotriazepinyl group, a tetrahydrotriazepinyl group, a perhydrotriazepinyl group, an azocinyl group, a dihydroazocinyl group, a tetrahydroazocinyl group, an oxohydroazocinyl group, a perhydroazocinyl group, a morphanyl group, a benzazocinyl group, an azepindolyl group, an indolinyl group, an indoleninyl group, an isoindolinyl group, an isonadoleninyl group, an indolyl group, a perhydroindolyl group, an isoindolyl group, a perhydroisoindolyl group, an indolizinyl group, an indolizidinyl group, an imidazopyridino group, an indazolyl group, a dihydroindazolyl group, a perhydroindazolyl group, a benzimidazolyl group, a dihydrobenzinaidazolyl group, a perhydrobenzimidazolyl group, a benzoxazolyl group, a dihydrobenzoxazolyl group, a perhydrobenzoxazolyl group, a benzothiazolyl group, a dihydrobenzothiazolyl group, a perhydrobenzothiazolyl group, a benzoxadiazolyl group, a benzothiadiazolyl group, a benzotriazolyl group, a purinyl group, a quinolyl group, a dihydroquinolyl group, a tetrahydroquinolyl group, a perhydroquinolyl group, a quinolizinyl group, a dihydroquinolizinyl group, a tetrahydroquinolizinyl group, an isoquinolinyl group, a dihydroisoquinolinyl group, a tetrahydroisoquinolinyl group, a perhydroisoquinolinyl group, a cinnolinyl group, a dihydrocinnolinyl group, a tetrahydrocinnolinyl group, a perhydrocinnolinyl group, a quinazolinyl group, a dihydroquinazolinyl group, a tetrahydroquinazolinyl group, a perhydroquinazolinyl group, a phthalazinyl group, a dihydrophthalazinyl group, a tetrahydrophthalazinyl group, a perhydrophthalazinyl group, a quinoxalinyl group, a dihydroquinoxalinyl group, a tetrahydroquinoxalinyl group, a perhydroquinoxalinyl group, a naphthyridinyl group, a dihydronaphthyridinyl group, a tetrahydronaphthyridinyl group, a perhydronaphthyridinyl group, a pteridinyl group, a quinolizidinyl group, a dihydrobenzoxazinyl group, a dihydrobenzothiazinyl group, a benzazepinyl group, a dihydrobenzazepinyl group, a tetrahydrobenzazepinyl group, a benzodiazepinyl group, a dihydrobenzodiazepinyl group, a tetrahydrobenzodiazepinyl group, a henzoxazepinyl group, a dihydrobenzoxazepinyl group, a tetrahydrobenzoxazepinyl group, a benzothiazepinyl group, a dihydrobenzothiazepinyl group, a tetrahydrobenzothiazepinyl group, a benzoxadiazepinyl group, a benzothiadiazepinyl group, a benzazepinyl group, a pyridoazepinyl group, a carbazolyl group, a dihydrocarbazolyl group, a tetrahydrocarbazolyl group, a perhydrocarbazolyl group, a β-carbolinyl group, a dihydro β-carbolinyl group, a tetrabydro β-carbolinyl group, a perhydro β-carbolinyl group, an acridinyl group, a dihydroacridinyl group, a tetrahydroacridinyl group, a perhydroacridinyl group, a phenazinyl group, a dihydrophenazinyl group, a tetrahydrophenazinyl group, a perhydrophenazinyl group, a phenothiazinyl group, a dihydrohydrophenothiazinyl group, a tetrahydrophenothiazinyl group, a perhydrophenothiazinyl group, a phenoxazinyl group, a dihydrophenoxazinyl group, a tetrahydropbenoxazinyl group, a perhydrophenoxazinyl group, a phenarsazinyl group, a phenanthridinyl group, a dihydrophenanthridinyl group, a tetrahydrophenanthridinyl group, a perhydrophenanthridinyl group, a phenanthrolinyl group, a dihydrophenanthrolinyl group, a tetrahydrophenanthrolinyl group, a perhydrophenanthrolinyl group, a perirnidinyl group, a dihydroperirnidinyl group, a tetrahydroperimidinyl group, a perhydroperimidinyl group, a pterinyl group, a pyrrolylidinyl group, a morphinanyl group, a hasubananyl group, a furyl group, a dihydrofuryl group, a tetrahydrofuryl group, a pyranyl group, a dihydropyranyl group, a tetrahydropyranyl group, an oxepinyl group, a dihydrooxepinyl group, a tetrahydrooxepinyl group, a perhydrooxepinyl group, a thienyl group, a dihydrothienyl group, a tetrahydrothienyl group, a thiopyranyl group, a dihydrothiopyranyl group, a tetrahydrothiopyranyl group, a thiepinyl group, a dihydrothiepinyl group, a tetrahydrothiepinyl group, a perhydrothiepinyl group, a benzofaryl group, a dihydrobenzofuryl group, a tetrahydrobenzofuryl group, a perhydrobenzofuryl group, an isobenzofuryl group, a dihydroisobenzofuryl group, a tetrahydroisobenzothryl group, a perhydroisobenzofuryl group, a benzothienyl group, a dihydrobenzothienyl group, a tetrahydrobenzothienyl group, a perhydrobenzothienyl group, an isobenzothienyl group, a dihydroisobenzothienyl group, a tetrahydroisobenzothienyl group, a perhydroisobenzothienyl group, a benzopyranyl group, a dihydrobenzopyranyl group, a perhydrobenzopyranyl group, a benzothiopyranyl group, a dihydrobenzotbiopyranyl group, a perhydrobenzothiopyranyl group, a benzoxepinyl group, a dihydrobenzoxepinyl group, a tetrahydrobenzoxepinyl group, a perhydrobenzoxepinyl group, a benzothiepinyl group, a dihydrobenzothiepinyl group, a tetrahydrobenzothiepinyl group, a perhydrobenzothiepinyl group, a henzofuryl group, a dihydrodibenzofuryl group, a tetrahydrodibenzofuryl group, a perhydrodibenzofuryl group, a xanthenyl group, a dihydroxanthenyl group, a tetrahydroxanthenyl group, a perhydroxanthenyl group, a benzothienyl group, a dihydrodibenzothienyl group, a tetrahydrodibenzothienyl group, a perhydrodihenzothienyl group, a thioxantenyl group, a dihydrothioxantenyl group, a tetrahydrothioxantenyl group, a perhydrothioxantenyl group, a phenoxathiinyl group, a dihydrophenoxathiinyl group, a tetrahydrophenoxathiinyl group, a perhydrophenoxathiinyl group, a dibenzodioxinyl group, a dihydrodibenzodioxinyl group, a tetrahydrodibenzodioxinyl group, a perhydrodibenzodioxinyl group, a thianthrenyl group, a dihydrothianthrenyl group, a tetrahydrothianthrenyl group, a perhydrothianthrenyl group, an oxiranyl group, an oxetanyl group, a thiiranyl group, a thietanyl group, an oxathiinyl group, a dihydrooxathiinyl group, a tetrahydrooxathiinyl group, a benzoxathiinyl group, a dihydrobenzoxathiinyl group, a tetrahydrobenzoxathiinyl group, a perhydrobenzoxathiinyl group, a benzodioxepanyl group, a dioxolanyl group, a dioxatiyl group, a dithiolanyl group, a dithianyl group, a dioxoindanyl group, a benzodioxanyl group, a chromanyl group, a benzodithiolanyl group, a benzodithianyl group, and in the case of an unsaturated heterocyclic group, a heterocyclic group in which at least a part is hydrogenated is also included.

Further, any two or three groups of the substituents R², R³, R⁴, R⁵, R⁶, and R⁷ each may be combined together to form a ring. Examples of the ring may include cyclopropane, cyclopropene, cyclohexane, cyclobutene, cyclopentane, cycloperitene, cyclopentadiene, cyclohexane, cyclohexene, cyclohexadiene, cycloheptane, cycloheptene, cycloheptadiene, cycloheptatriene, cyclooctane, cyclooetene, cyclooctadiene, cyclooctatriene, aziridine, azetidine, diazetidine, pyrrolidine, piperidine, homopiperidine, pyrazolidine, imidazolidine, triazolidine, tetrazolidine, oxazolidine, isoxazolidine, thiazolidine, isothiazolidine, oxazodiazolidine, thiadiazolidine, piperazine, homopiperazine, triazepane, morpholine, thiomorpholine, quinuclidine, tropane, pyrroline, pyrazoline, imidazoline, oxazoline, thiazoline, isoxazoline, isothiazoline, dihydrooxazole, tetrahydrooxazole, dihydroisooxazole, tetrahydroisooxazole, dihydrothiazole, tetrahydrothiazole, dihydroisothiazole, tetrahydroisothiazole, triazotine, dihydrooxadiazole, tetrahydrooxadiazole, dihydrothiadiazole, tetrahydrothiadiazole, dihydrofurazan, tetrahydrofurazan, piperideine, triazinane, dihydropyridine, tetrahydropyridine, dihydropyrazine, tetrahydropyrazine, dihydropyrimidine, tetrahydropyrimidine, perhydropyrimidine, dihydropyridazine, tetrahydropyridazine, perhydropyridazine, oxazine, dihydro oxazine, tetrahydro oxazine, oxadiazine, dihydro oxadiazine, tetrahydro oxadiazine, thiazine, dihydrothiazine, tetrahydrothiazine, thiadiazine, dihydrothiadiazine, tetrahydrothiadiazine, dihydroazepine, tetrahydroazepine, perhydroazepine, dihydrodiazepine, tetrahydrodiazepine, perhydrodiazepine, oxazepine, dihydrooxazepine, tetrahydrooxazepine, perhydrooxazepine, oxadiazepine, dihydrooxadiazepine, tetrahydrooxadiazepine, perhydrooxadiazepine, thiazepine, dihydrothiazepine, tetrahydrothiazepine, perhydrothiazepine, thiadiazepine, dihydrothiadiazepine, tetrahydrothiadiazepine, perhydrothiadiazepine, triazepine, dihydrotriazepine, tetrahydrotriazepine, perhydrotriazepine, azocine, dihydroazocine, tetrahydroazocine, oxohydroazocine, perhydroazocine, morphan, azepindole, indoline, indolenine, isoindoline, isoindolenine, perhydroindole, perhydroisoindole, perhydroisoindole, indolizidine, dihydroindazole, perhydroindazole, dihydrohenzimidazole, perhydrohenzimidazole, dihydrobenzoxazole, perhydrobenzoxazole, dihydrobenzothiazole, perhydrobenzothiazole, dihydroquinoline, tetrahydroquinoline, perhydroquinoline, quinolizine, dihydroquinolizine, tetrahydroquinolizine, dihydroisoquinoline, tetrahydroisoquinoline, perhydroisoquinoline, dihydrocinnoline, tetrahydrocinnoline, perhydrocinnoline, dihydroquinazoline, tetrahydroquinazoline, perhydroquinazoline, dihydrophthalazine, tetrahydrophthalazine, perhydrophthalazine, dihydroquinoxaline, tetrahydroquinoxaline, perhydroquinoxaline, dihydronaphthyridine, tetrahydronaphthyridine, perhydronaphthyridine, quinolizidine, dihydrobenzoxazine, dihydrobenzothiazine, dihydrobenzazepine, tetrahydrobenzazepine, perhydrobenzazepine, dihydrobenzodiazepine, tetrahydrobenzodiazepine, petbydrobenzodiazepine, dihydrobenzoxazepine, tetrahydrobenzoxazepine, perhydrobenzoxazepine, dihydrobenzothiazepine, tetrahydrobenzothiazepine, perhydrobenzothiazepine, dihydrocarbazole, tetrahydrocarbazole, perhydrocarbazole, dihydro β-carboline, tetrahydro β-carboline, perhydro β-carboline, dihydroacridine, tetrahydroacridine, perhydroacridine, dihydrophenazine, tetrahydrophenazine, perhydrophenazine, dihydrohydrophenothiazine, tetrahydrophenothiazine, perhydrophenothiazine, dihydrophenoxazine, tetrahydrophenoxazine, perhydrophenoxazine, dihydrophenanthridine, tetrahydrophenanthridine, perhydrophenanthridine, dihydrophenanthroline, tetrahydrophenanthroline, perhydrophenanthroline, dihydroperimidine, tetrahydroperimidine, perhydroperimidine, pyrrolizidine, morphinan, hasubanan, dihydrofuran, tetrahydrofuran, pyran, dihydropyran, tetrahydropyran, dihydrooxepin, tetrahydrooxepin, perhydrooxepin, dihydrothiophene, tetrahydrothiophene, thiopyran, dihydrothiopyran, tetrahydrothiopyran, dihydrothiepine, tetrahydrothiepine, perhydrothiepine, dihydrobenzofuran, tetrahydrobenzofuran, perhydrobenzofuran, dihydroisobenzofuran, tetrahydroisobenzofuran, perhydroisohenzofuran, dihydrobenzothiophene, tetrahydrobenzothiophene, perhydrobenzothiophene, dihydroisobenzothiophene, tetrahydrobenzothiophene, perhydrobenzothiophene, benzopyran, dihydrobenzopyran, perhydroherizopyran, benzothiopyran, dihydrobenzothiopyran, perhydrobenzothiopyran, dihydrobenzoxepin, tetrahydrobenzoxepin, perhydrobenzoxepin, dihydrobenzothiepine, tetrahydrobenzothiepine, perhydrobenzothiepine, dihydrodibenzofuran, tetrahydrodibenzofuran, perhydrodibenzofuran, xanthene, dihydroxanthene, tetrahydroxanthene, perhydroxanthene, dihydrodibenzothiophene, tetrahydrodibenzothiophene, perhydrodibenzothiophene, thioxanthene, dihydrothioxanthene, tetrahydrothioxanthene, perhydrothioxanthene, dihydrophenoxathiin, tetrahydrophenoxathiin, perhydrophenoxathiin, dihydrodibenzodioxin, tetrahydrodibenzodioxin, perhydrodibenzodioxin, dihydrothianthrene, tetrahydrothianthrene, perhydrothianthrene, oxirane, oxetane, thiirane, thietane, dihydrooxathiin, tetrahydrooxathiin, dihydrobenzoxathiin, tetrahydrohenzoxathiin, perhydrobenzoxathiin, beuzodioxepane, dioxolan, dioxane, dithiolane, dithiane, dioxoindane, berizodioxane, chromane, benzodithiolane, benzodithiane, and in the ease of an unsaturated ring, a ring in which at least a part is hydrogenated is also included. Further, in the case of forming a ring, it is preferable that any two substituents of R³, R⁴, R⁵, R⁶, and R⁷ form a ring.

Further, examples of the substituent which the alkyl group, the cycloalkyl group, the alkenyl group, the cycloalkenyl group, the alkynyl group, the aromatic group, and the heterocyclic group may include groups selected from a hydroxyl group, an alkyl group, a cycloalkyl group, an alkenyl group, a cycloalkenyl group, an alkynyl group, a halogen atom, an aromatic group, a heterocyclic group, an alkoxy group, a guanidine group, an alkylthio group, an alkoxycarbonyl group, an aryloxy group, an arylthio group, an acyl group, a substituted sulfonyl group, a heterocyclyloxy group, a heterocyclyl thio group, an amide group, a ureido group, a carboxy group, a carbamoyl group, an oxo group, a thioxo group, a sulfamoyl group, a sulfo group, a cyano group, a nitro group, an acyloxy group, an azido group, a sulfonamide group, a mercapto group, an alkoxycarbonyl amino group, an aminocarbonyloxy group, a substituted sulfinyl group, a sulfamide group, an aminosulfonyloxy group, an alkoxysulfonyl amino group, a substituted sulfonyloxy group, an alkoxycarbonyl group, an alkoxycarbonyloxy group, an alkoxysulfonyl group, an imino group which may be substituted, an azo group which may be substituted, an Rx(Ry)N group, and an Rx(Ry)(Rz)N⁺ group. Herein, Rx, Ry, and Rz each independently represent a hydrogen atom, an alkyl group, a cycloalkyl group, an alkenyl group, a cycloalkenyl group, an alkynyl group, an aromatic hydrocarbon group, or a heterocyclic group. Further, two or more of Rx, Ry, and Rz may combine together to form a saturated or unsaturated hetero ring, and this ring can also form a condensed ring or a spire ring with an aliphatic ring or a hetero ring and can also form a condensed ring with an aromatic ring.

Incidentally, Rx, Ry, Rz excluding the case of a hydrogen atom and the alkyl group, the cycloalkyl group, the alkenyl group, the cycloalkenyl group, the alkynyl group, the aromatic group, and the heterocyclic group as the substituent which are described herein include the same groups as the groups represented by R¹, R², R³, R⁴, R⁵, R⁶, and R⁷. Further, the alkyl group of the alkoxy group and the alkylthio group as substituents has the same definition as the definition of the alkyl group in R¹, R², R³, R⁴, R⁵, R⁶, and R⁷ described above, and the aryl group of the aryloxy group and the arylthio group has the same definition as the definition of the aromatic group in R¹, R², R³, R⁴, R⁵, R⁶, and R⁷ described above.

Further, examples of a guanidine group, an acyl group, a substituted sulfonyl group, a heterocyclyloxy group, a heterocyclyl thio group, a carbamoyl group, a ureido group, an amide group, a sulfamoyl group, an acyloxy group, a sulfonamide group, an alkoxycarbonyl amino group, an aminocarbonyloxy group, a substituted sulfinyl group, a sulfamide group, an aminosulfonyloxy group, an alkoxysulfonyl amino group, a substituted sulfonyloxy group, an alkoxycarbonyl group, an alkoxycarbonyloxy group, and an alkoxysulfonyl group as substituents are as follows.

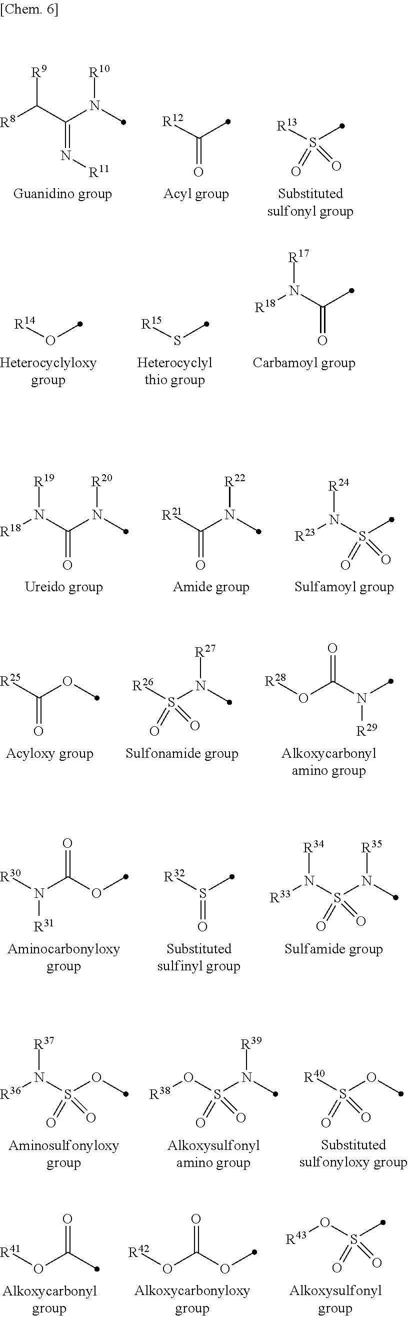

In the exemplary groups, R⁸, R⁹, R¹⁰, R¹¹, R¹², R¹⁶, R¹⁷, R¹⁸, R¹⁹, R²⁰, R²¹, R²², R²³, R²⁴, R²⁵, R²¹, R²⁹, R³⁰, R³¹, R³², R³³, R³⁴, R³⁵, R³⁶, R³⁷, R³⁹, and R⁴⁰ represent a hydrogen atom, a substituted or unsubstituted alkyl group, a substituted or unsubstituted cycloalkyl group, a substituted or unsubstituted alkenyl group, a substituted or unsubstituted cycloalkenyl group, a substituted or unsubstituted alkynyl group, a substituted or unsubstituted aromatic group, or a substituted or unsubstituted heterocyclic group. R¹³, R²⁶, R²⁸, R³⁸, R⁴¹, R⁴², and R⁴³ represent a substituted or unsubstituted alkyl group, a substituted or unsubstituted cycloalkyl group, a substituted or unsubstituted alkenyl group, a substituted or unsubstituted cycloalkenyl group, a substituted or unsubstituted alkynyl group, a substituted or unsubstituted aromatic group, or a substituted or unsubstituted heterocyclic group. R¹⁴ and R¹⁵ represent a substituted or unsubstituted heterocyclic group. Further, examples of substituents of those substituted alkyl group, substituted cycloalkyl group, substituted alkenyl group, substituted cycloalkenyl group, substituted alkynyl group, substituted aromatic group, and substituted heterocyclic group include the same substituents as substituents of those groups in R¹, R², R³, R⁴, R⁵, R⁶, and R⁷ described above.

The groups represented by R¹, R², R³, R⁴, R⁵, R⁶, and R⁷ are preferably each independently a hydrogen atom, a substituted or unsubstituted linear or branched chain alkyl group having carbon number of 1 to 6, a substituted or unsubstituted cycloalkyl group having carbon number of 3 to 8, a substituted or unsubstituted linear or branched alkenyl group having carbon number of 2 to 6, substituted or unsubstituted cycloalkenyl group having carbon number of 3 to 8, a substituted or unsubstituted linear or branched alkynyl group having carbon number of 2 to 6, a substituted or unsubstituted monocyclic or polycyclic aromatic group having carbon number of 6 to 14, or a substituted or unsubstituted 3- to 8-membered heterocyclic group containing at least one of a nitrogen atom, an oxygen atom, or a sulfur atom as a ring-constituting atom. Among them, the groups represented by R¹, R², R³, R⁴, R⁵, R⁶, and R⁷ each independently are a hydrogen atom or an alkyl group having carbon number of 1 to 6, or two of R³, R⁴, R⁵, R⁶, and R⁷ are coupled to form a cycloalkyl group having carbon number of 3 to 8, which is preferable in terms of ease of availability of a raw material. Particularly, it is preferable that both of R¹ and R² are a hydrogen atom or one of R¹ and R² is a methyl group.

In the tertiary amine compound or imine compound-polymer conjugate represented by Formula (I) or (II) and the amine form that is an important intermediate represented by Formula (III) or (V), D⁺ is a structure in which the tertiary amine compound or imine compound D forms a quaternary ammonium salt or an iminium salt, and D specifically represents a compound represented by the following Formula (XII).

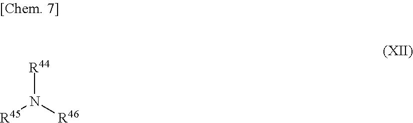

R⁴⁴, R⁴⁵, and R⁴⁶ each independently are a substituted or unsubstituted alkyl group, a substituted or unsubstituted cycloalkyl group, a substituted or unsubstituted alkenyl group, a substituted or unsubstituted cycloalkenyl group, a substituted or unsubstituted alkynyl group, a substituted or unsubstituted aromatic group, or a substituted or unsubstituted heterocyclic group, an R⁴⁷O— group, an R⁴⁸S— group, or an R⁴⁹(R⁵⁰)N— group (herein, R⁴⁷, R⁴⁸, R⁴⁹, and R⁵⁰ each independently are a substituted or unsubstituted alkyl group, a substituted or unsubstituted cycloalkyl group, a substituted or unsubstituted alkenyl group, a substituted or unsubstituted cycloalkenyl group, a substituted or unsubstituted alkynyl group, a substituted or unsubstituted aromatic group, a substituted or unsubstituted heterocyclic group, or an Rx(Ry)N group) two of R⁴⁴, R⁴⁵, and R⁴⁶ may combine together to form a double bond so as to form an amino group or azo group with N at the center, or at least two of R⁴⁴, le, and R⁴⁶ may be bonded to each other to form a single bond or a double bond so as to form a saturated or unsaturated hetero ring, the ring can also form a condensed ring or a Spiro ring with an aliphatic ring or a hetero ring, and a condensed ring can also be formed with an aromatic ring. The alkyl group, the cycloalkyl group, the alkenyl group, the cycloalkenyl group, the alkynyl group, the aromatic group, or the heterocyclic group described herein has the same meaning as definition in R¹, R², R⁴, R⁵, R⁶, and R⁷ described above. Further, Rx and Ry described herein have the same meaning as definitions of Rx and Ry in the Rx(Ry)N group that is the substituent of R¹, R², R³, R⁴, R⁵, R⁶, and R⁷ described above.

Examples of the saturated or unsaturated hetero ring formed by R⁴⁴, and R⁴⁶ being bonded to each other may include aziridine, azetidine, diazetidine, pyrrolidine, piperidine, homopiperidine, pyrazolidine, imidazolidine, triazolidine, tetrazolidine, oxazolidine, isoxazolidine, thiazolidine, isothiazolidine, oxadiazolidine, thiadiazolidine, piperazine, homopiperazine, triazepane, morpholine, thiomorpholine, quinuclidine, tropane, pyrroline, pyrazoline, imidazoline, oxazoline, thiazoline, isooxazoline, isothiazoline, pyrrol, imidazole, pyrazole, oxazole, dihydrooxazole, tetrahydrooxazole, isooxazole, dihydroisooxazole, tetrahydroisooxazole, triazole, dihydrothiazole, tetrahydrothiazole, isothiazole, dihydroisothiazole, tetrahydroisothiazole, triazoline, triazole, oxodiazole, dihydrooxodiazole, tetrahydrooxodiazole, thiadiazole, dihydrothiadiazole, tetrahydrothiadiazole, tetrazoline, tetrazole, furazan, dihydrofurazan, tetrahydrofurazan, piperideine, triazinane, pyridine, dihydropyridine, tetrahydropyridine, pyrazine, dihydropyrazine, tetrahydropyrazine, pyrimidine, dihydropyrimidine, tetrahydropyrimidine, perhydropyrimidine, pyridazine, dihydropyridazine, tetrahydropyridazine, perhydropyridazine, triazine, dihydrotriazine, tetrahydrotriazine, oxazine, dihydro oxazine, tetrahydro oxazine, oxadiazine, dihydro oxadiazine, tetrahydro oxadiazine, thiazine, dihydrothiazine, tetrahydrothiazine, thiadiazine, dihydrothiadiazine, tetrahydrothiadiazine, azepine, dihydroazepine, tetrahydroazepine, perhydroazepine, diazepine, dihydrodiazepine, tetrahydrodiazepine, perhydrodiazepine, oxazepine, dihydrooxazepine, tetrahydrooxazepine, perhydrooxazepine, oxadiazepine, dthydrooxadiazepine, tetrahydrooxadiazepine, perhydrooxadiazepine, thiazepine, dihydrothiazepine, tetrahydrothiazepine, perhydrothiazepine, thiadiazepine, dihydrothiadiazepine, tetrahydrothiadiazepine, perhydrothiadiazepine, triazepine, dihydrotriazepine, tetrahydrotriazepine, perhydrotriazepine, azocine, dihydroazocine, tetrahydroazocine, oxohydroazocine, perhydroazocine, morphan, benzazocine, azepindole, indoline, indolenine, isoindoline, isoindolenine, indole, perhydroindole, isoindole, perhydroisoindole, indolizine, indolizidine, imidazopyridine, indazole, dihydroindazole, perhydroindazole, benzimidazole, dihydrobenzimidazole, perhydrobenzimidazole, benzoxazole, dihydrobenzoxazole, perhydrobenzoxazole, benzothiazole, dihydrobenzothiazole, perhydrobenzothiazole, benzoxadiazole, benzothiadiazole, benzotriazole, purine, quinoline, dihydroquinoline, tetrahydroquinoline, perhydroquinoline, dihydroquinolizine, tetrahydroquinolizine, isoquinoline, dihydroisoquinoline, tetrahydroisoquinoline, perhydroisoquinoline, cinnoline, dihydrocinnoline, tetrahydrocinnoline, perhydrocinnoline, quinazoline, dihydroquinazoline, tetrahydroquinazoline, perhydroquinazoline, phthalazine, dihydrophthalazine, tetrahydrophthalazine, perhydrophthalazine, quinoxaline, dihydroquinoxaline, tetrahydroquinoxaline, perhydroquinoxaline, naphthyridine, dihydronaphthyridine, tetrahydronaphthyridine, perhydronaphthyridine, pteridine, quinolizidine, dihydrobenzoxazine, dihydrobenzothiazine, benzazepine, dihydrobenzazepine, tetrahydrobenzazepine, benzodiazepine, dihydrohenzodiazepine, tetrahydrobenzodiazepine, benzoxazepine, dihydrobenzoxazepine, tetrahydrobenzoxazepine, benzothiazepine, dihydrobenzothiazepine, tetrahydrobenzothiazepine, benzoxadiazepine, benzothiazeazepine, benzazepine, pyridoazepine, carbazole, dihydrocarbazole, tetrahydrocarbazole, perhydrocarbazole, β-carboline, dihydro β-carboline, tetrahydro β-carboline, perhydro β-carboline, acridine, dihydroacridine, tetrahydroacridine, perhydroacridine, phenazine, dihydrophenazine, tetrahydrophenazine, perhydrophenazine, phenothiazine, dihydrohydrophenothiazine, tetrahydrophenothiazine, perhydrophenothiazine, phenoxazine, dihydrophenoxazine, tetrahydropherioxazine, perhydrophenoxazine, phenarsazine, phenanthridine, dihydrophenanthridine, tetrahydrophenanthridine, perhydrophenanthridine, phenanthroline, dihydrophenanthroline, tetrahydrophenanthroline, perhydrophenanthroline, perimidine, dihydroperimidine, tetrahydroperimidine, perhydroperimidine, pterin, pyrrolizidine, morphinan, hasubanan, and pyridinomorpholine, and in the case of an unsaturated hetero ring, a hetero ring in which at least a part is hydrogenated is also included. Further, a structure in which two or more of those structures are bonded to each other directly or via an alkylene group can also be employed, and the heterocyclic group has the same definition as that of the heterocyclic group represented by R¹, R², R³, R⁴, R⁵, R⁶, and R⁷ described above and can have a substituent. A specific structure is not particularly limited as long as it has a structure of a tertiary amine or imine compound and can form an ammonium salt or an iminium salt, but it is preferable that a structure having a 4-cyanoguanidinopyridine skeleton, α-(2,4-difluorophenyl)-5-fluoro-β-methyl-α-(1H-1,2,4-triazol-1-ylmethyl)-4-pyrimidine ethanol, 2,5-anhydro-1,3,4-trideoxy-2-C-(2,4-difluorophenyl)-4-[[4-[4-[4-[1-(1-ethyl-2-hydroxypropyl)-1,5-dihydro-5-oxo-4H-1,2,4-triazol-4-yl]phenyl]-1-piperazinyl]phenoxy]methyl]-1-(1H-1,2,4-triazol-1-yl)pentitol, 2,5-anhydro-1,3,4-trideoxy-2-C-(2,4-difluorophenyl)-4-[[4-[4-[4-[1-[(1S,2S)-1-ethyl-2-hydroxypropyl]-1,5-dihydro-5-oxo-4H-1,2,4-triazol-4-yl]phenyl]-1-piperazinyl]phenoxy]methyl]-1-(1H-1,2,4-triazol-1-yl)-D-Threo-pentitol (posaconazole), or 4-[2-[(1R,2R)-2-(2,5-difluorophenyl)-2-hydroxy-1-methyl]-3-(1H-1,2,4-triazol-1-yl)propyl]-4-thiazolyl]-benzonitrile (isavuconazole) is not employed.

Examples of a substituent which the alkyl group, the cycloalkyl group, the alkenyl group, the cycloalkenyl group, the alkynyl group, the aromatic group, the heterocyclic group, the R⁴⁷O— group, the R⁴⁸S— group, or the R⁴⁹(R⁵⁰)N— group, and the saturated or unsaturated hetero ring formed by R⁴⁴, R⁴⁵, and R⁴⁶ being bonded to each other may have include the same substituents of those groups in R¹, R², R³, R⁴, R⁵, R⁶, and R⁷ described above.

Further, D⁺ is a structure in which the tertiary amine compound or imine compound D forms a quaternary ammonium salt or an iminium salt. The tertiary amine compound or imine compound D is preferably a compound having bioactivity. Examples of the compound having bioactivity may include active ingredients of a medical drug, a quasi-drug, a medical device, an in-vitro diagnostic medical drug, a regenerative medical product, a medical drug for animals, an agricultural chemical, a supplement, and the like. If the released tertiary amine compound or imine compound D has bioactivity and can form a quaternary ammonium salt or iminium salt structure, the structure of the tertiary amine compound or imine compound D is not limited, and a known compound which can be used as a compound having bioactivity can be used.

In the amine form represented by Formula (III) or (V), X⁻ is a counter anion of the quaternary ammonium salt or iminium salt in D⁺, and examples thereof include a halide ion such as a chloride ion, a bromide ion, and an iodide ion; an inorganic acid anion such as a sulfate ion and a nitrate ion; and an organic acid anion such as a trifluoroacetate ion, a methanesulfonate ion, a toluenesulfonate ion, and a trifluoromethanesulfonate ion. Further, the amine form represented by Formula (III) or (V) may form a salt with an inorganic acid or an organic acid. Examples of the inorganic acid include hydrochloric acid, sulfuric acid, and nitric acid, and examples of the organic acid include trifluoroacetic acid, methanesulfonic acid, toluenesulfonic acid, benzenesulfonic acid, and trifluoromethanesulfonic acid. Regarding a salt formed with an inorganic acid or an organic acid, preferably, an amino group existing at the molecule terminal of the amine form represented by Formula (III) or (V) forms a salt with an inorganic acid or an organic acid.

The polymer providing a structure derived from the polymer having a carboxy group has at least one carboxy group in the molecule and can be represented by Formula (IV).

Poly-CO₂H (IV)

Hereinafter, the moiety of Poly is also referred to as “polymer residue”, in some cases. The polymer may be a naturally derived polymer or artificially synthesized polymer. The artificially synthesized polymer may be, for example, a polymer obtained by polymerizing monomers having a carboxy group or one in which a carboxy group is introduced into a polymer originally having no carboxy group by chemical modification. Further, in a case where the polymer having a carboxy group has a plurality of carboxy groups, the amine form represented by Formula (III) or (V) may be formed by condensing the plurality of carboxy groups in the conjugate.

For example, as an embodiment of the present invention, in a case where the polymer having a carboxy group has a plurality of carboxy groups, a conjugate in which the amine form is bonded to three or more carboxy groups in the polymer is exemplified, and a conjugate in which the amine form is bonded to 11 or more carboxy groups in the polymer can be more preferably exemplified.

For example, as an embodiment of the present invention, the conjugate can also be represented by the following Formula (XX) and this is equivalent to the compound represented by Formula W.

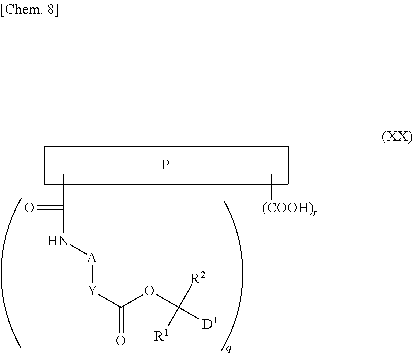

(in Formula (XX), D⁺, R¹, R², Y, and A are as defined above, and

a moiety represented by

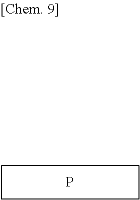

is a polymer (polymer chain) excluding a group derived from a carboxy group (a group represented by —C(═O)NH-A-Y—C(═O)OCR¹R²D⁺) and a carboxy group; q is the number of compounds condensed with the polymer (amine forms represented by Formula (III)) (that is, the number of groups derived from carboxy group); and r is the number of carboxy groups).

Further, the total of q and r is the total number of carboxy groups in the polymer represented by Formula (IV) (for example, 25 or more and 25000 or less), the number of each of q and r is not particularly limited, but q is preferably three or more and more preferably 11 or more.

Incidentally, the above Formula (XX) does not mean only that q groups represented by —C(═O)NH-A-Y—C(═O)OCR¹R²D⁺ and r groups represented by —COOH are continuously arranged in a block form in the polymer chain. It should be understood that the groups represented by —C(═O)NH-A-Y—C(═O)OCR¹R²D⁺ and the groups represented by —COOH may be randomly arranged in the polymer chain or may be arranged in a block form or regularly arranged in an alternating manner.

Further, as an embodiment of the present invention, the conjugate can also be represented by the following Formula (XXX) and this is equivalent to the compound represented by Formula (II).

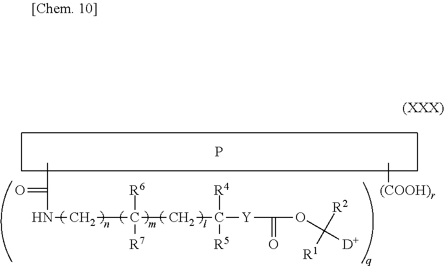

(in Formula (XXX), D⁺, Y, R¹, R², R⁴, R⁵, R⁷, r, and P are as defined above; and q is the number of compounds condensed with the polymer (amine forms represented by Formula (V))).

The values of q and r are determined according to the ratio of the compound (the amine form represented by Formula (III) or (V)) to be condensed to the polymer having a carboxy group. The degree to which the amine form represented by the above Formula (III) or (V) is condensed to the polymer having a carboxy group can be appropriately changed and adjusted depending on the types of the compound (amine form) having a structure represented by D⁺ and the polymer having a carboxy group. The degree of introduction of the compound having a structure represented by D can be indicated as “introduction ratio” in the present specification. The introduction ratio can be obtained by methods such as calculation of the integration ratio by ¹H NMR or calculation of concentration by spectroscopy. Examples of the spectroscopy include ultraviolet-visible absorption spectroscopy. In the present specification, the introduction ratio (mol %) obtained by calculation of the molar ratio, the introduction ratio (wt %) obtained by concentration calculation, and the like are collectively simply referred to as the “introduction ratio” in some cases. The introduction ratio based on calculation of the molar ratio (calculation of integration ratio by ¹H NMR) is not particularly limited, but a range of 1 to 80 mol % can be exemplified. Herein, q and r are preferably values that fall within the range of the introduction ratio described above.

The carboxy group of the polymer remaining without being condensed to the amine form represented by Formula (III) or (V) may exist as a free carboxy group, may faint a salt using a metal such as lithium, sodium, potassium, magnesium, or calcium, or an organic base such as triethylamine, tributylamine, or pyridine, or may form a salt using tetrabutylammonium hydroxide.

Examples of the polymer having a carboxy group include synthetic polymers such as polyacrylic acid, polymethacrylic acid, polymaleic acid, polylactic acid (PLA), polyglycolic acid (PGA), lactic acid-glycolic acid copolymer (PLGA), polycaprolactone, polycarboxyisopropylacrylamide, polyethylene terephthalate, polybutylene terephthalate, and carboxy group-modified polyethylene glycol; polysaccharides which also exist in nature such as alginic acid, hyaluronic acid, heparin, chondroitin, chondroitin sulfate (A, B, C, D, and E), keratan sulfate, heparan sulfate, dermatan sulfate, pectin (homogalacturonan and rhamnogalacturonan), xanthane gum, xylan, and sacran; semisynthetic polymers in which a carboxy group is introduced into polysaccharides such as carboxymethyl cellulose, carboxymethyl chitin, carboxymethyl chitosan, carboxymethyl dextran, carboxymethyl amylose, and succinyl chitosan; polyamino acids such as polyaspartic acid, polyglutamic acid, and protein; and nucleic acids such as deoxyribonucleic acid into which a carboxy group is introduced. Among these, from the viewpoint of solubilization of poorly soluble tertiary amine compound or imine compound, biocompatibility, and the like, in the case of selecting a polymer, a water-soluble polymer having a carboxy group is preferable, particularly, polysaccharides are preferable, and glycosaminoglycan is most preferable.

Examples of a water-soluble polymer having a carboxy group include synthetic polymers such as polyacrylic acid, polymethacrylic acid, polymaleic acid, polycarboxyisopropylacrylamide, and carboxy group-modified polyethylene glycol; polysaccharides which also exist in nature such as alginic acid, hyaluronic acid, heparin, chondroitin, chondroitin sulfate (A, B, C, D, and E), keratan sulfate, heparan sulfate, dermatan sulfate, pectin (homogalacturonan and rhamnogalacturonan), xanthane gum, xylan, and sacran; semisynthetic polymers in which a carboxy group is introduced into polysaccharides such as carboxymethyl cellulose, carboxymethyl chitin, carboxymethyl chitosan, carboxymethyl dextran, carboxymethyl amylose, and succinyl chitosan; polyamino acids such as polyaspartic acid, polyglutamic acid, and protein; nucleic acids such as deoxyribonucleic acid into which a carboxy group is introduced.

Examples of the polysaccharides include polysaccharides which also exist in nature such as alginic acid, hyaluronic acid, heparin, chondroitin, chondroitin sulfate (A, B, C, D, and E), keratin sulfate, heparan sulfate, dermatan sulfate, pectin (homogalacturonan and rhamnogalacturonan), xanthane gum, xylan, and sacran; and semisynthetic polymers in which a carboxy group is introduced into polysaccharides such as carboxymethyl cellulose, carboxymethyl chitin, carboxymethyl chitosan, carboxymethyl dextran, carboxymethyl amylose, and succinyl chitosan.

Examples of the glycosaminoglycan include hyaluronic acid, heparin, chondroitin, chondroitin sulfate (A, B, C, D, and E), keratan sulfate, heparan sulfate, and dermatan sulfate. When the tertiary amine compound or imine compound is conjugated with chondroitin sulfate via the aminoalkoxy carbonyloxymethyl group linker according to the present invention, as shown in Test Example 1 described later, the release rate of the tertiary amine compound or imine compound tends to be lowered.

Those polymers having a carboxy group may be further modified or cross-linked by various methods in some cases. Further, these polymers having a carboxy group may form a pharmaceutically acceptable salt, for example, a salt with a metal such as lithium, sodium, potassium, magnesium, or calcium or an organic base such as triethylamine, tributylamine and pyridine, or a salt may be formed using tetrabutylammonium hydroxide.

The weight average molecular weight of these exemplified polymers is not particularly limited, but, for example, in the case of hyaluronic acid, preferred examples of the weight average molecular weight may include 10,000 or more and 10,000,000 or more, 500,000 or more and 5,000,000 or less, preferably 600,000 or more and 3,000,000 or less, and more preferably 600,000 or more and 1,200,000 or less.

Further, the tertiary amine compound or imine compound-polymer conjugate in the present invention is also not particularly limited, but a tertiary amine compound or imine compound-polymer conjugate in which the weight average molecular weight of the polymer is 500,000 or more and 5,000,000 or less and the introduction ratio is 1 to 80% can be mentioned as a preferred example.

Poly means a partial structure of the polymer represented by the Formula (IV) excluding a carboxy group moiety used for condensation with an amine form represented by Formula (III) or (V). As Poly, a water-soluble polymer residue, a polysaccharide residue, a glycosaminoglycan residue, a chondroitin residue, a chondroitin sulfate residue, and a hyaluronic acid residue can be exemplified as a preferable aspect. These each mean a partial structure of water-soluble polymer, polysaccharide, glycosaminoglycan, chondroitin, chondroitin sulfate, and hyaluronic acid excluding a carboxy group condensed with a compound (III) or (V).

A production example of the tertiary amine compound or imine compound-polymer conjugate represented by Formula (I) is as follows:

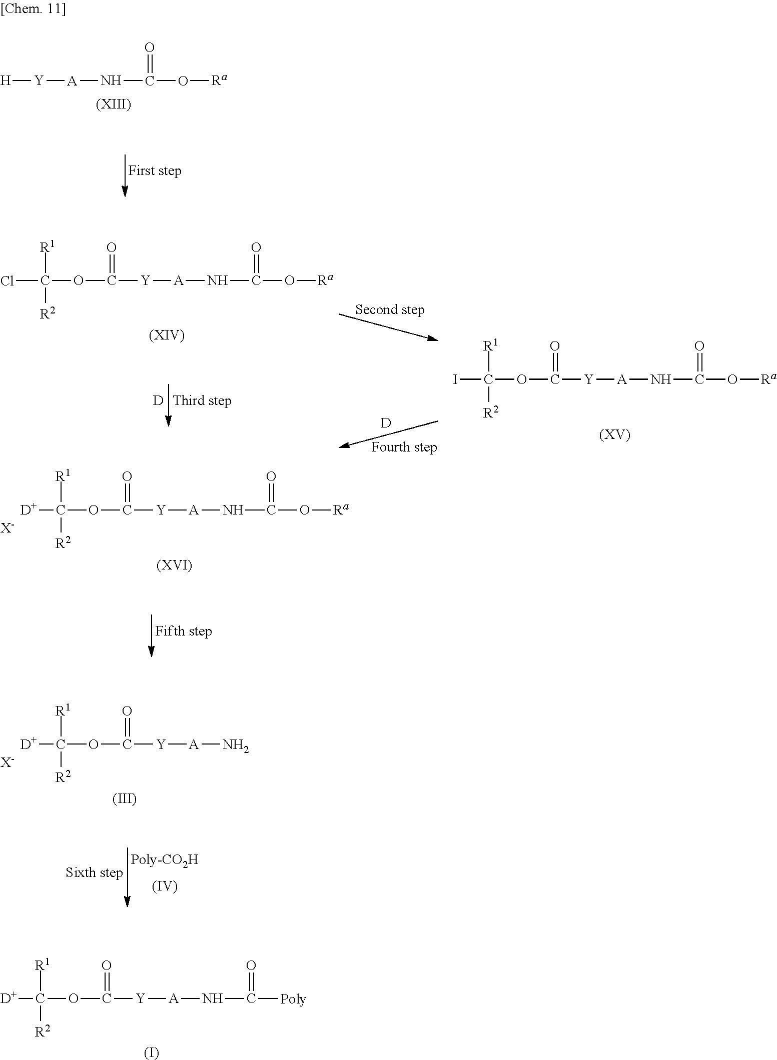

(in the formula, R^{a} represents a benzyl group or a t-butyl group, and R¹, R², D⁺, X⁻, Y, A, and Poly are as defined above).

First Step

This step is to produce the chloromethyl ester form represented by the above Formula (XIV) from the protected amine form represented by the above Formula (XIII). At this time, in a case where Y in the above Formula (XIII) is an oxygen atom, a carbonic ester bond is provided as a product represented by the above Formula (XIV), and in a case where Y in the above Formula (XIII) is a nitrogen atom, a urethane bond is provided as the product. This step can be performed by reacting chloroalkyl chloroformate with the protected amino acid represented by the above Formula (XIII) in the presence of a base.

Upon performing this step, this step is preferably performed in a solvent, for example, an organic solvent such as methylene chloride, chloroform, dichloroethane, ethyl acetate, acetone, benzene, toluene, xylene, dimethylformamide, acetonitrile, tetrahydrofuran, dioxane, diethyl ether, diisopropyl ether, or dimethoxyethane can be used, and if necessary, water can also be added. As chloroalkyl chloroformate, for example, chloromethyl chloroformate, 1-chloroethyl chloroformate, 1-chloro-2-methylpropyl chloroformate, or the like can be used. Further, as the base, for example, organic bases such as pyridine, N,N-diisopropylethylamine, triethylamine, 2,6-lutidine, 4-dimethylaminopyridine, diazabicycloundecene, diazabicyclononene, and 1,8-bis(dimethylamino)naphthalene and inorganic bases such as sodium hydrogen carbonate, sodium carbonate, potassium bicarbonate, potassium carbonate, sodium hydroxide, potassium hydroxide, and cesium carbonate can be used. As the reaction temperature, the step can proceed usually in a range of −78° C. to 200° C. and preferably in a range of −20° C. to 80° C.

Second Step

This step is to produce the iodomethyl ester form represented by the above Formula (XV) by iodizing the chloromethyl ester form represented by the above Formula (XIV). As an iodizing agent to be used in this step, for example, sodium iodide, potassium iodide, or the like can be used.

Upon performing this step, this step is preferably performed in a solvent, and for example, an organic solvent such as ethyl acetate, acetone, benzene, toluene, xylene, dimethylformamide, acetonitrile, tetrahydrofuran, dioxane, diethyl ether, diisopropyl ether, or dimethoxyethane can be used. As the reaction temperature, the step can proceed usually in a range of 0° C. to 200° C. and preferably in a range of 10° C. to 150° C. Further, if necessary, calcium chloride can also be added.

Third Step

This step is to produce the quaternary ammonium salt or iminium salt represented by the above Formula (XVI) by reacting the chloromethyl ester form represented by the above Formula (XIV) with the tertiary amine compound or imine compound represented by D.

Upon performing this step, this step can be performed in an organic solvent or in the absence of a solvent. As the organic solvent, for example, methylene chloride, chloroform, dichloroethane, ethyl acetate, acetone, benzene, toluene, xylene, dimethylformamide, acetonitrile, tetrahydrofuran, dioxane, diethyl ether, diisopropyl ether, dimethoxyethane, methanol, ethanol, 1-propanol, 2-propanol, or the like can be used. As the reaction temperature, the step can proceed usually in a range of 0° C. to 200° C. and preferably in a range of 20° C. to 150° C.

Fourth Step

This step is to produce the quaternary ammonium salt or iminium salt represented by the above Formula (XVI) by reacting the iodomethyl ester represented by the above Formula (XV) with the tertiary amine compound or imine compound represented by D.

Upon performing this step, this step can be performed in an organic solvent or in the absence of a solvent. As the organic solvent, for example, methylene chloride, chloroform, dichloroethane, ethyl acetate, acetone, benzene, toluene, xylene, dimethylformamide, acetonitrile, tetrahydrofuran, dioxane, diethyl ether, diisopropyl ether, dimethoxyethane, methanol, ethanol, 1-propanol, 2-propanol, or the like can be used. As the reaction temperature, the step can proceed usually in a range of 0° C. to 200° C. and preferably in a range of 10° C. to 100° C.

Further, in this step, the reaction can also proceed while the iodomethyl ester form represented by the above Formula (XV) is not isolated but is generated in the reaction system. That is, the chloromethyl ester form represented by the above Formula (XIV) can also be reacted with the tertiary amine compound or imine compound represented by D in the presence of an iodizing agent. In this case, as the iodizing agent, for example, sodium iodide, potassium iodide, or the like can be used, and as the solvent, acetone, acetonitrile, dioxane, tetrahydrofuran, toluene, ethyl acetate, dimethylformamide, dimethoxyethane, or the like can be used. As the reaction temperature, the step can proceed usually in a range of 0° C. to 200° C. and preferably in a range of 10° C. to 150° C. Further, if necessary, calcium chloride can also be added.

Fifth Step

This step is to produce the amine form represented by the above Formula (III) by deprotecting the quaternary ammonium salt or iminium salt represented by the above Formula (XVI).

In this step, in a case where R^{a} represents a benzyl group, the quaternary ammonium salt or iminium salt is deprotected by catalytic hydrogen addition so that the amine form represented by the above Formula (HI) can be produced. For example, a platinum catalyst such as platinum oxide or platinum carbon, a palladium catalyst such as palladium carbon, palladium black, or palladium oxide, or a nickel catalyst such as Raney nickel can be used. Upon performing this step, this step is preferably performed in a solvent, and for example, methanol, ethanol, 2-propanol, tetrahydrofuran, dimethylformamide, dioxane, water, or the like can be used. As the reaction temperature, the step can proceed usually in a range of −50° C. to 200° C. and preferably in a range of 10° C. to 100° C.

In this step, in a case where R^{a} represents a t-butyl group, the amine form represented by Formula (III) can be produced by deprotection using an acid. As the acid, for example, hydrogen chloride, hydrochloric acid, sulfuric acid, nitric acid, methanesulfonic acid, benzenesulfonic acid, p-toluenesulfonic acid, trifluoromethanesuofonic acid, trifluoroacetic acid, or the like can be used. The amine form represented by Formula (III) which is obtained in this step is produced by forming salts with those acids. In this step, the reaction can proceed in the absence of a solvent or in a solvent, and as the solvent, for example, ethyl acetate, dioxane, methanol, ethanol, 1-propanol, 2-propanol, water, or the like can be used. As the reaction temperature, the step can proceed usually in a range of −50° C. to 200° C. and preferably in a range of 0° C. to 120° C.

Sixth Step

This step is to produce the tertiary amine compound or imine compound-polymer conjugate represented by the above Formula (I) by condensing the amine form represented by the above Formula (III) with the polymer having a carboxy group represented by the above Formula (IV), As the condensing agent to be used herein, for example, 1-ethyl-3-(3-dimethylaminopropyl)carbodiimide hydrochloride (EDC or WSC), 4-(4,6-dimethoxy-1,3,5-triazin-2-yl)-4-methylmorpholinium chloride (DMT-MM), tetramethylfluoroformamidinium hexafluorophosphate (TFFH), bis(tetramethylene)fluoroformamidinium hexafluorophosphate (BTFFH), or the like can be used. Further, in a case where the carboxy group of the polymer having a carboxy group is derivatized into an active ester such as N-hydroxysuccinimide ester or p-nitrophenyl ester, it is not necessary to add a condensing agent, and condensation can also be performed by only mixing with the amine form represented by Formula (III), or if necessary, adding a base.

This step is preferably performed in a solvent, and for example, water or an organic solvent such as methylene chloride, chloroform, dichloroethane, toluene, ethyl acetate, acetone, dimethylformamide, formamide, N-methylpyrrolidone, acetonitrile, tetrahydrofuran, dioxane, diethyl ether, dimethoxyethane, dimethyl sulfoxide, methanol, ethanol, 1-propanol, 2-propanol, n-butanol, isobutanol, Cert-butanol, ethylene glycol can be used. In the case of using a polymer having a carboxy group as a water-soluble polymer in this step, a solvent containing at least a protic solvent is preferable, and a solvent containing at least water is more preferable. The mixing ratio of an aprotic solvent and a protic solvent in a mixed solvent can be an arbitrary ratio, and the ratio thereof is not particularly limited, but the ratio of the aprotic solvent:the protic solvent is preferably in a range of 0:100 to 90:1.0 (weight ratio), In a preferred embodiment, regarding a solvent to be used, the ratio of the organic solvent:water is in a range of 0:100 to 90:10 (weight ratio).

Herein, examples of the aprotic solvent include acetone, dimethylformamide, formamide, N-methylpyrrolidone, acetonitrile, tetrahydrofuran, dioxane, dimethoxyethane, and dimethyl sulfoxide.

Further, examples of the protic solvent include water and lower alcohols having carbon number of 1 to 4 such as methanol, ethanol, propanol, 2-propanol, n-butanol, isobutanol, and test-butanol. Of the protic solvents, water is preferable,

This step is more specifically a step of producing a conjugate represented by the following Formula (I), the step including a step of condensing a compound represented by the following Formula (III) and a polymer having a carboxy group represented by the following Formula (IV),

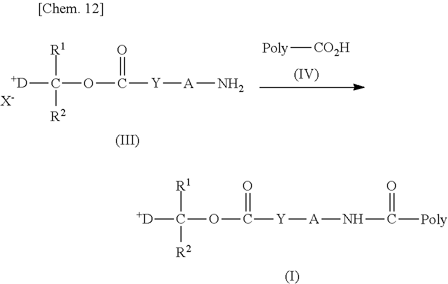

[in Formulae (I), (III), and (IV), D⁺, R¹, R², Y, A, and Poly are as defined above; X⁻ is a counter anion of D⁺, and the compound represented by Formula (III) may form a salt with an inorganic acid or an organic acid].

This step is more specifically a step for producing a conjugate represented by the following Formula (II), the step including a step of condensing a compound represented by the following Formula (V) and a polymer having a carboxy group represented by the following Formula (IV).

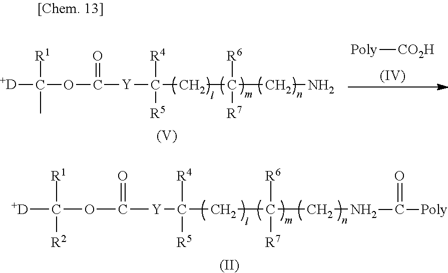

[in Formulae (II), (IV), and (V), D⁺, R¹, R², R⁴, R⁵, R⁶, R⁷, Y, l, m, n, and Poly are as defined above; X⁻ is a counter anion of D⁺, and the compound represented by Formula (V) may form a salt with an inorganic acid or an organic acid].

As compared to a case where the tertiary amine compound or the imine compound is bonded to the water-soluble polymer bonded with the linker, by bonding the compound represented by the above Formula (III) or (V) prepared in advance is bonded to the water-soluble polymer, the conjugate can be efficiently synthesized.

A still another aspect of the present invention is a method for producing a conjugate, the method including a step of bonding a tertiary amine compound containing a nitrogen atom capable of forming a quaternary ammonium salt or an imine compound capable of forming an iminium salt to a polymer having a carboxy group via a linker represented by the following Formula (VI):

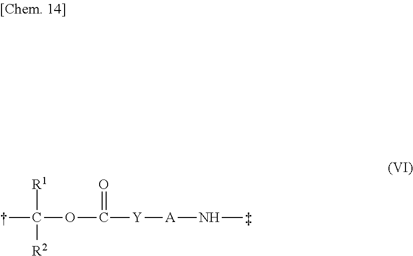

(wherein, R¹, R², Y, and A are as defined above, symbol † is a node with the nitrogen atom forming the quaternary ammonium salt or the iminium salt, and symbol ‡ represents a node with a carbonyl carbon atom derived from the carboxy group of the polymer).

The conjugate can be obtained by the method exemplified in the above-described steps 1 to 6 using the linker represented by Formula (VI). Therefore, a further another aspect of the present invention is a method for producing the compound represented by Formula (I) using the linker represented by Formula (VI), the method including a step of bonding a tertiary amine compound containing a nitrogen atom capable of forming a quaternary ammonium salt or an imine compound capable of forming an iminium salt to a polymer having a carboxy group via the linker. The linker is more specifically represented by the following Formula (VII):

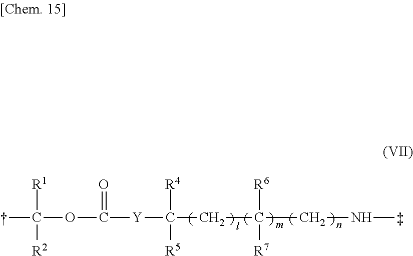

(wherein, R¹, R², R⁴, R⁵, R⁶, R⁷, Y, l, m, and n in the above (VII) are as defined above, symbol † is a node with the nitrogen atom forming the quaternary ammonium salt or the iminium salt, and symbol ‡ represents a node with a carbonyl carbon atom derived from the carboxy group of the polymer).

As described above, the “linker” has a structure for bonding a polymer serving as a carrier to a bioactive substance, particularly, for bonding a tertiary amine compound containing a nitrogen atom capable of forming a quaternary ammonium salt or an imine compound capable of forming an iminium salt to a polymer having a carboxy group.

Properties of the tertiary amine compound or imine compound-polymer conjugate represented by Formula (I) or (II) are not particularly limited, but, for example, in the case of an aqueous solution, it is preferable to have high filtering properties. With a tertiary amine compound or imine compound-polymer conjugate having such properties, an aqueous solution having high filtering properties (for example, a solution formulation) can be prepared.

From the viewpoint of having high filtering properties (for example, being easily filtered even in a case where the amount of the tertiary amine compound or imine compound D existing is large), in the case of selecting a polymer used in a conjugate, glycosaminoglycan or a salt thereof is preferably used, chondroitin, chondroitin sulfate, hyaluronic acid, or a salt thereof is more preferably used, and chondroitin sulfate, hyaluronic acid, or a salt thereof is most preferably used.

The tertiary amine compound or imine compound-polymer conjugate disclosed in the present invention is a conjugate whose release rate can be controlled by releasing a drug and is expected to be used in medical drugs and the like, as clearly shown from Test Example described later.

The present invention relates to inventions specified by the following items.

1. A compound represented by Formula (I) or a pharmaceutically acceptable salt thereof;

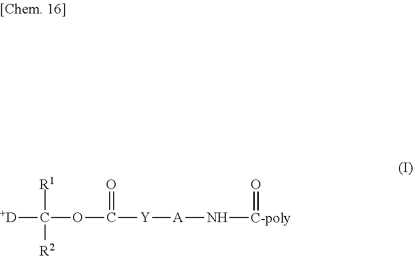

[in Formula (I), D⁺ is a structure forming a quaternary ammonium salt or an iminium salt from a tertiary amine compound or an imine compound D; a nitrogen atom of D⁺ forming the quaternary ammonium salt or the iminium salt and a carbon atom to which R¹ and R² bond are bonded to each other; Y is O or NR³; R¹, R², and R³ are each independently a hydrogen atom, a substituted or unsubstituted alkyl group, a substituted or unsubstituted cycloalkyl group, a substituted or unsubstituted alkenyl group, a substituted or unsubstituted cycloalkenyl group, a substituted or unsubstituted alkynyl group, a substituted or unsubstituted aromatic group, or a substituted or unsubstituted heterocyclic group; A is a substituted or unsubstituted bivalent hydrocarbon group, may contain one or more heteroatoms at a position except for both ends which are bonded to —Y— or —NH—, and the heteroatoms are each independently selected from the group consisting of —O—, —NH— which may have a substituent, and —S—; any two or three groups of R¹, R², R³, and A may combine together to form a ring; and Poly is a polymer residue and —C(═O)— adjacent to Poly is derived from any carboxy group of the polymer].

2. The compound according to the above 1. or a pharmaceutically acceptable salt thereof, wherein the above Formula (I) is represented by the following Formula (II);

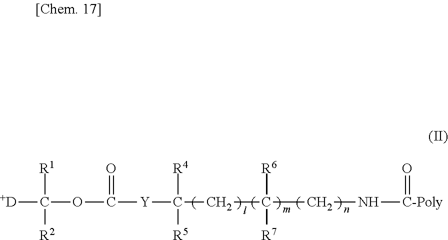

[in Formula (II), D⁺, R¹, R², Y, and Poly are as defined in the above 1; R⁴, R⁵, R⁶, and R⁷ are each independently a hydrogen atom, a substituted or unsubstituted alkyl group, a substituted or unsubstituted cycloalkyl group, a substituted or unsubstituted alkenyl group, a substituted or unsubstituted cycloalkenyl group, a substituted or unsubstituted alkynyl group, a substituted or unsubstituted aromatic group, or a substituted or unsubstituted heterocyclic group; any two or three groups of R¹, R², R³, R⁴, R⁵, R⁶, and R⁷ may combine together to form a ring; 1 and n are each independently are 0, 1, or 2; and m is 0 or 1],

3. The compound according to the above 1. or 2. or a pharmaceutically acceptable salt thereof, wherein in Formula (I) or (II), R¹, R², R³, R⁴, R⁵, R⁶, and R⁷ are each independently a hydrogen atom, a substituted or unsubstituted linear or branched chain alkyl group having carbon number of 1 to 6, a substituted or unsubstituted cycloalkyl group having carbon number of 3 to 8, a substituted or unsubstituted linear or branched alkenyl group having carbon number of 2 to 6, substituted or unsubstituted cycloalkenyl group having carbon number of 3 to 8, a substituted or unsubstituted linear or branched alkynyl group having carbon number of 2 to 6, a substituted or unsubstituted monocyclic or polycyclic aromatic group having carbon number of 6 to 14, or a substituted or unsubstituted 3- to 8-membered heterocyclic group containing at least one of a nitrogen atom, an oxygen atom, or a sulfur atom as a ring-constituting atom.
4. The compound according to any one of the above 1. to 3. or a pharmaceutically acceptable salt thereof, wherein in Formula (I) or (II), a substituent of alkyl group, a substituent of cycloalkyl group, a substituent of alkenyl group, a substituent of cycloalkenyl group, a substituent of alkynyl group, a substituent of aromatic group, and a substituent of heterocyclic group in the groups represented by R¹, R², R³, R⁴, R⁵, R⁶, and R⁷ are groups selected from a hydroxyl group, an alkyl group, a cycloalkyl group, an alkenyl group, a cycloalkenyl group, an alkynyl group, a halogen atom, an aromatic group, a heterocyclic group, an alkoxy group, a guanidino group, an alkylthio group, an alkoxycarbonyl group, an aryloxy group, an arylthio group, an acyl group, a substituted sulfonyl group, a heterocyclyloxy group, a heterocyclylthio group, an amide group, a ureido group, a carboxy group, a carbamoyl group, an oxo group, a thioxo group, a sulfamoyl group, a sulfo group, a cyano group, a nitro group, an acyloxy group, an azido group, a sulfonamide group, a mercapto group, an alkoxycarbonyl amino group, an aminocarbonyloxy group, a substituted sulfinyl group, a sulfamide group, fan aminosulfonyloxy group, an alkoxysulfonyl amino group, a substituted sulfonyloxy group, an alkoxycarbonyl group, an alkoxycarbonyloxy group, an alkoxysulfonyl group, an amino group which may be substituted, an azo group which may be substituted, an Rx(Ry)N group, and an Rx(Ry)(Rz)N⁺ group, Rx, Ry, and Rz are each independently selected from the group consisting of a hydrogen atom, an alkyl group, a cycloalkyl group, an alkenyl group, a cycloalkenyl group, an alkynyl group, an aromatic hydrocarbon group, and a heterocyclic group, and at this time, two or more of Rx, Ry, and Rz may combine together to form a saturated or unsaturated hetero ring.
5. The compound according to any one of the above 1. to 4. or a pharmaceutically acceptable salt thereof, wherein in Formula (I) or (II), Poly is a water-soluble polymer residue.
6. The compound according to any one of the above 1. to 4. or a pharmaceutically acceptable salt thereof, wherein in Formula (I) or (II), Poly is a polysaccharide residue.
7. The compound according to any one of the above 1. to 4. or a pharmaceutically acceptable salt thereof, wherein in Formula (I) or (II), Poly is a glycosaminoglycan residue.
8. The compound according to any one of the above 1. to 4. or a pharmaceutically acceptable salt thereof, wherein in Formula (I) or (II), Poly is a residue of chondroitin, chondroitin sulfate or hyaluronic acid.
9. The compound according to any one of the above 1. to 8. or a pharmaceutically acceptable salt thereof, wherein a counter anion of D⁺ is an inorganic acid anion or an organic acid anion.
10. A compound represented by the following Formula (XX) or a pharmaceutically acceptable salt thereof;

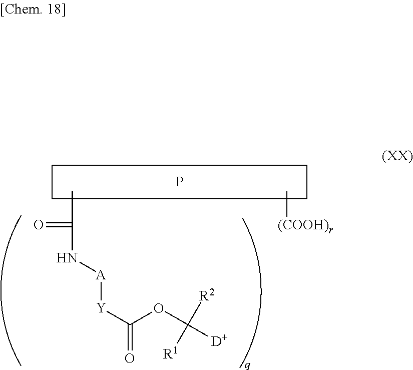

[in Formula (XX), D⁺, R¹, R², Y, and A are as defined in 1, and

a moiety represented by

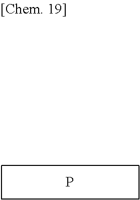

is a polymer excluding a group derived from a carboxy group (a group represented by —C(═O)NH-A-Y—C(═O)OCR¹R²D⁺) and a carboxy group; q is the number of compounds condensed with the polymer; and r is the number of carboxy groups].
11. The compound according to the above 10. or a pharmaceutically acceptable salt thereof, wherein the compound represented by the above Formula (XX) is a compound represented by the following Formula (XXX):

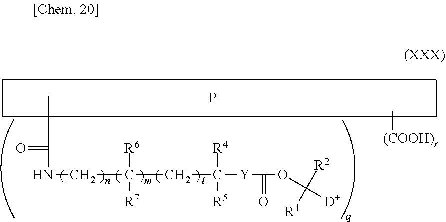

[in Formula (XXX), D⁺, R¹, and R² are as defined in the above 1; R⁴, R⁵, R⁶, and R⁷ are as defined in the above 2; and q and r are as defined in the above 10, and

a moiety represented by

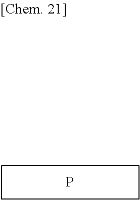

is a polymer excluding a group derived from a carboxy group and a carboxy group].
12. A method for producing a compound represented by the following Formula (I) or a pharmaceutically acceptable salt thereof, the method comprising a step of condensing a compound represented by the following Formula (III) and a polymer having a carboxy group represented by the following Formula (IV):

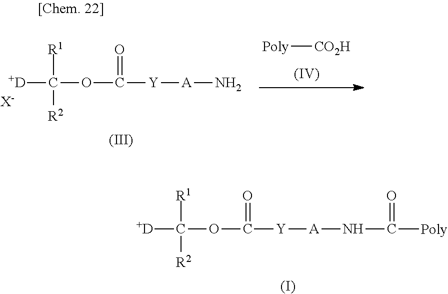

[in Formulae (I), (III), and (IV), D⁺, Y, A, R¹, R², and Poly are as defined in the above 1; X⁻ is a counter anion of and the compound represented by Formula (III) may form a salt with an inorganic acid or an organic acid].

13. The production method according to the above 12, wherein the compound represented by the above Formula (III) is a compound represented by the following Formula (V), and the compound represented by the above Formula (I) is a compound represented by the following Formula (II):

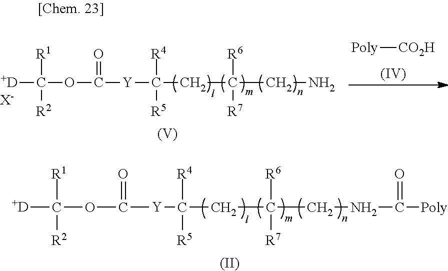

[in Formulae (II), (IV), and (V), D⁺, Y, R¹, R², and Poly are as defined in 1; R⁴, R⁵, R⁶, R⁷, l, n, and m are as defined in the above 2; X⁻ is a counter anion of D⁺, and the compound represented by Formula (V) may form a salt with an inorganic acid or an organic acid].

14. A method for producing a conjugate, the method comprising a step of bonding a tertiary amine compound containing a nitrogen atom capable of forming a quaternary ammonium salt or an imine compound capable of forming an iminium salt to a polymer having a carboxy group via a linker represented by the following Formula (VI):

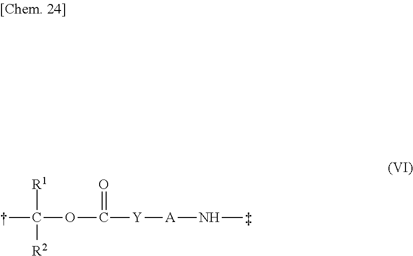

(wherein, R¹, R², Y, and A in the above (VI) are as defined in the above 1, symbol † is a node with the nitrogen atom forming the quaternary ammonium salt or the iminium salt, and symbol ‡ represents a node with carbonyl carbon derived from the carboxy group of the polymer).

15. The method for producing a conjugate according to the above 14, wherein the linker is represented by the following Formula (VII):

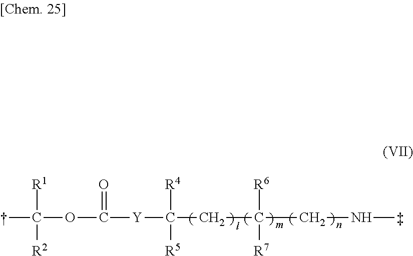

(wherein, Y, R¹, R², R⁴, R⁵, R⁶, R⁷, l, m, and n in the above (VII) are as defined in the above 2, symbol † is a node with the nitrogen atom forming the quaternary ammonium salt or the iminium salt, and symbol ‡ represents a node with carbonyl carbon derived from the carboxy group of the polymer).

### EXAMPLES

Hereinafter, the present invention will be described in detail by means of Reference Examples and Examples; however, the scope of the present invention is not limited to these specific examples.

### Reference Example 1

### Carbonic acid chloromethyl 3-[[(1,1-dimethylethoxy)carbonyl]amino]propyl ester

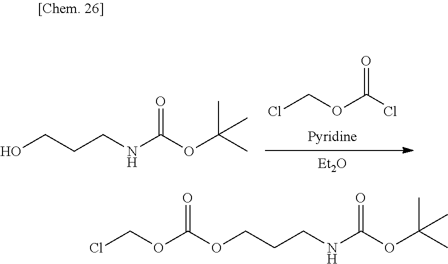

A diethyl ether solution of 1.77 g (13.69 mmol) of chloromethyl chloroformate was added at −15° C. to a diethyl ether solution of 2.00 g (11.41 mmol) of N-(3-hydroxypropyl)carbamic acid 1,1-dimethylethyl ester, and subsequently, a diethyl ether solution of 1.08 g (13.69 mmol) of pyridine was added dropwise. The reaction solution was warmed to room temperature and stirred overnight, water was then added to the reaction solution, and the resultant liquid was extracted with diethyl ether. The obtained organic layer was washed with saturated ammonium chloride aqueous solution, saturated sodium bicarbonate water, and saturated saline and dried over anhydrous magnesium sulfate, and the solvent was distilled off under reduced pressure. The residue was purified by silica gel column chromatography (6→9% ethyl acetate/hexane) to obtain 2.02 g (66%) of the title compound.

¹H-NMR (CDCl₃, δ): 1.44 (9H, s), 1.90 (2H, quin, J=7 Hz), 3.23 (2H, q, J=7 Hz), 4.30 (2H, t, J=7 Hz), 4.68 (1H, br-s), 5.73 (2H, s)

### Reference Example 2

### 3-[[[[3-[[(1,1-dimethylethoxy)carbonyl]amino]propoxy]carbonyl]oxy]methyl]-2-methyl-1-[(2,3,4,9-tetrahydro-9-methyl-4-oxo-1H-carbazol-3-yl)methyl]-1H-imidazolium chloride

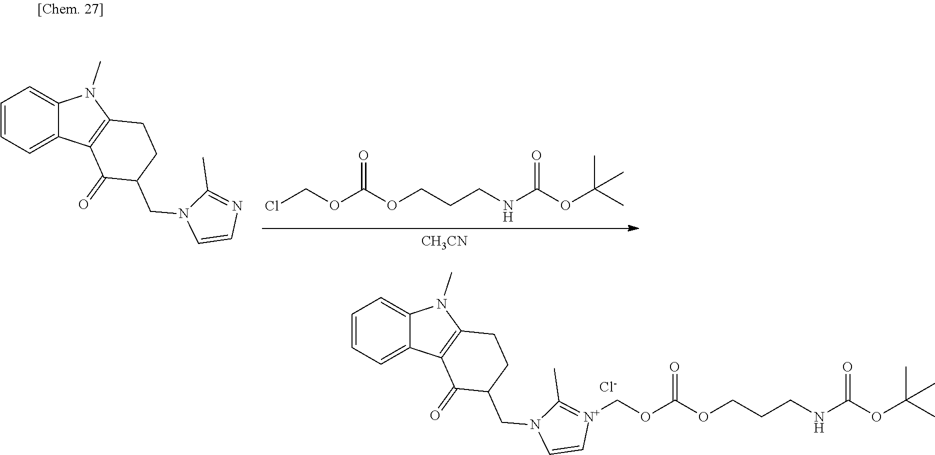

413 mg (1.41 mmol) of ondansetron was added at room temperature to an acetonitrile solution of 565 mg (2.11 mmol) of carbonic acid chloromethyl 3-[[(1,1-dimethylethoxy)carbonyl]amino]propyl ester and stirred al 100° C. overnight. The reaction solution was condensed in a water bath set to 40° C. The residue was purified by silica gel column chromatography (10→15% methanol/chloroform) to obtain 267 mg (34%) of the title compound.

¹H-NMR (CDCl₃, δ): 1.42 (9H, s), 1.87 (2H, quip, J=6 Hz), 2.06 (1H, qd, J=12, 5 Hz), 2.77-2.80 (1H, m), 3.05 (3H, s), 3.08-3.36 (5H, m), 3.70 (3H, s), 4.25 (2H, t, J=6 Hz), 4.57 (1H, dd, J=14, 6 Hz), 4.74 (1H, br-s), 4.78 (1H, dd, J=14, 6 Hz), 6.15 (1H, d, J=12 Hz), 6.20 (1H, d, J=12 Hz), 7.26-7.45 (3H, m), 7.44 (1H, d, J=2 Hz), 7.88 (1H, d, J=2 Hz), 8.10-8.12 (1H, m)

### Reference Example 3

### 3-[[[(3-aminopropoxy)carbonyl]oxy]methyl]-2-methyl-1-[(2,3,4,9-tetrahydro-9-methyl-4-oxo-1H-carbazol-3-yl)methyl]-1H-imidazolium chloride hydrochloride

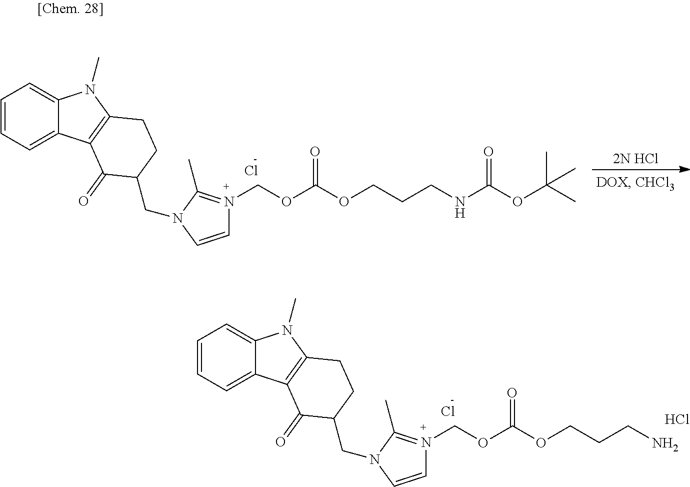

2 mL of 4 N hydrochloric acid/dioxane solution was added under cooling on ice to 2 mL of chloroform solution of 267 mg (0.48 mmol) of 3-[[[[3-[[(1,1-dimethylethoxy)carbonyl]amino]propoxy]carbonyl]oxy]methyl]-2-methyl-1-[(2,3,4,9-tetrahydro-9-methyl-4-oxo-1H-carbazol-3-yl)methyl]-1H-imidazolium chloride. The reaction solution was warmed to room temperature and left to stand still for 30 minutes. Thereafter, the solvent was distilled off under reduced pressure. Ethyl acetate was added to the residue and stirred and washed. Crystals were collected by filtration to obtain 228 mg (96%) of the title compound.

¹H-NMR (DMSO-d6, δ): 1.91-2.03 (3H, m), 2.17-2.22 (1H, m), 2.78 (3H, s), 2.81-3.20 (5H, m), 3.75 (3H, s), 4.24 (2H, t, J=6 Hz), 4.34 (1H, dd, J=14, 7 Hz), 4.72 (1H, dd, J=14, 7 Hz), 6.15 (1H, d, J=12 Hz), 6.17 (1H, d, J=12 Hz), 7.22 (1H, td, J=8, 1 Hz), 7.27 (1H, td, J=8, 1 Hz), 7.56 (1H, d, J=8 Hz), 7.81 (1H, d, J=3 Hz), 7.84 (1H, d, J=3 Hz), 7.99 (1H, d, J=8 Hz), 8.13 (3H, br-s)

### Example 1

### [3-[[[(ondansetron)methoxy]carbonyl]oxy]propyl]amino-chondroitin sulfate conjugate

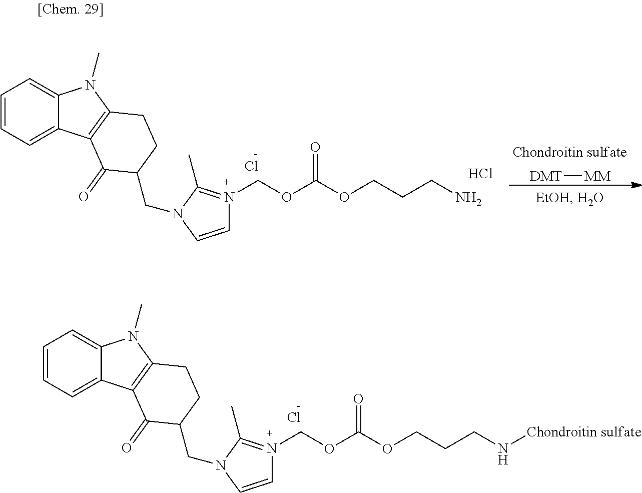

2 mL of ethanol was slowly added dropwise under stirring to 4.0 g (0.398 mmol) of 5% sodium chondroitin sulfate aqueous solution. To the mixed liquid, a solution of 40 mg (0.080 mmol) of 3-[[[(3-aminopropoxy)carbonyl]oxy]methyl]-2-methyl-1-[(2,3,4,9-tetrahydro-9-methyl-4-oxo-1H-carbazol-3-yl)methyl]-1H-imidazolium chloride hydrochloride in 1 mL of ethanol was added, a solution of 38 mg (0.136 mmol) of 4-(4,6-dimethoxy-1,3,5-triazin-2-yl)-4-methylmorpholinium chloride (DMT-MM) in 1 mL of ethanol was then added, 1 mL of ethanol and 1 mL of water were further added, and the resultant liquid was stirred at room temperature overnight. 100 μL of 20% sodium chloride aqueous solution was added to the reaction solution, and ethanol was further added dropwise (2 mL) until immediately before the reaction solution became cloudy. The reaction solution was added dropwise under stirring to 12 mL of 93% ethanol, and 5 mL of ethanol was added to the mixed liquid and stirred for 1 hour. Precipitates were isolated using a centrifuge, washed twice with 90% ethanol, twice with ethanol, and twice with diethyl ether. The obtained precipitates were dried overnight using a vacuum pump to obtain 202 mg of the title compound, Based on values of integral in ¹H-NMR, the introduction ratio of ondansetron per whole disaccharide unit (glucuronic acid) of chondroitin sulfate was 20 mol %.

### Reference Example 4

### Carbonic acid chloromethyl 3-[[(1,1-dimethylethoxy)carbonyl]amino]-1-methylpropyl ester

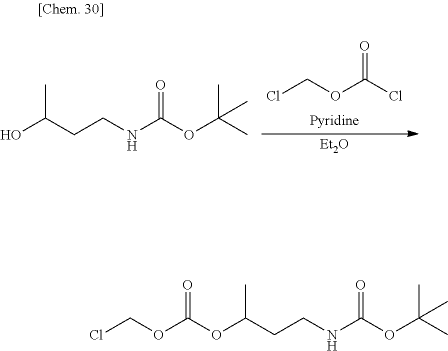

A diethyl ether solution (2 mL) of 633 mg (4.91 mmol) of chloromethyl chloroformate was added at −15° C. to a diethyl ether (10 mL) solution of 774 ma (4.09 mmol) of N-(3-hydroxybutyl)carbamic acid 1,1-dimethyl ethyl ester, and subsequently, a diethyl ether (4 solution of 338 mg (4.91 mmol) of pyridine was added dropwise. After the reaction solution was warmed to room temperature and stirred overnight, a diethyl ether (8 mL) solution of 633 mg (4.91 mmol) of chloromethyl chloroformate and 338 mg (4.91 mmol) of pyridine was added dropwise again at −15° C., and the resultant liquid was stirred at room temperature overnight. Water was added to the reaction solution, and the resultant liquid was extracted with diethyl ether. The obtained organic layer was washed with saturated ammonium chloride aqueous solution, saturated sodium bicarbonate water, and saturated saline and dried over anhydrous magnesium sulfate, and the solvent was distilled off under reduced pressure. The residue was purified by silica gel column chromatography (6→9% ethyl acetate/hexane) to obtain 883 mg (77%) of the title compound.

¹H-NMR (CDCl₃, δ): 1.35 (3H, d, J=7 Hz), 1.44 (9H, s), 1.81 (2H, q, J=7 Hz), 3.08-3.15 (1H, m), 3.25-3.29 (1H, m), 4.71 (1H, br-s), 4.92 (1H, seat, J=7 Hz), 5.72 (1H, d, J=7 Hz), 5.73 (1H, d, J=7 Hz)

### Reference Example 5

### 3-[[[[3-[[(1,1-dimethylethoxy)carbonyl]amino]-1-methylpropoxy]carbonyl]oxy]methyl]-2-methyl-1-[(2,3,4,9-tetrahydro-9-methyl-4-oxo-1H-carbazol-3-yl)methyl]-1H-imidazolium chloride

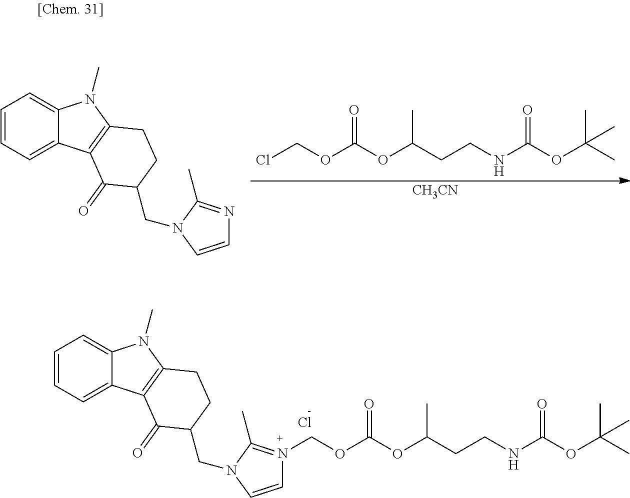

701 mg (2.39 mmol) of ondansetron was added at room temperature to an acetonitrile solution of 449 mg (1.59 mmol) of carbonic acid chloromethyl 3-[[(1,1-dimethylethoxy)carbonyl]amino]-1-methylpropyl ester and stirred at 85° C. overnight. The reaction solution was condensed in a water bath set to 40° C. The residue was purified by silica gel column chromatography (10-420% methanol/chloroform) to obtain 729 mg (80%) of the title compound.

¹H-NMR (CDCl₃, δ): 1.33 (3H, d, J=6 Hz), 1.41 (9H, s), 1.74-1.83 (2H, m), 2.06 (1H, qd, J=12, 6 Hz), 2.77-2.80 (1H, m), 3.04 (3H, s), 3.10-3.37 (5H, m), 3.70 (3H, s), 4.55 (1/2H, dd, J=14, 6 Hz), 4.56 (1/2H, dd, J=14, 6 Hz), 4.72 (1H, br-s), 4.79 (1H, dd, J=14, 6 Hz), 4.84-4.85 (1H, m), 6.12 (1/2H, d, J=12 Hz), 6.13 (1/2H, d, J=12 Hz), 6.16 (1/2H, d, J=12 Hz), 6.17 (1/2H, d, J=12 Hz), 7.26-7.33 (3H, m), 7.43 (1/2H, d, J=2 Hz), 7.44 (1/2H, d, J=2 Hz), 7.87 (1/2H, d, J=2 Hz), 7.88 (1/2H, d, J=2 Hz), 8.10-8.11 (1H, m)

### Reference Example 6

### 3-[[[(3-amino-1-methylpropoxy)carbonyl]oxy]methyl]-2-methyl-1-[(2,3,4,9-tetrahydro-9-methyl-4-oxo-1H-carbazol-3-yl)methyl]-1H-imidazolium chloride hydrochloride

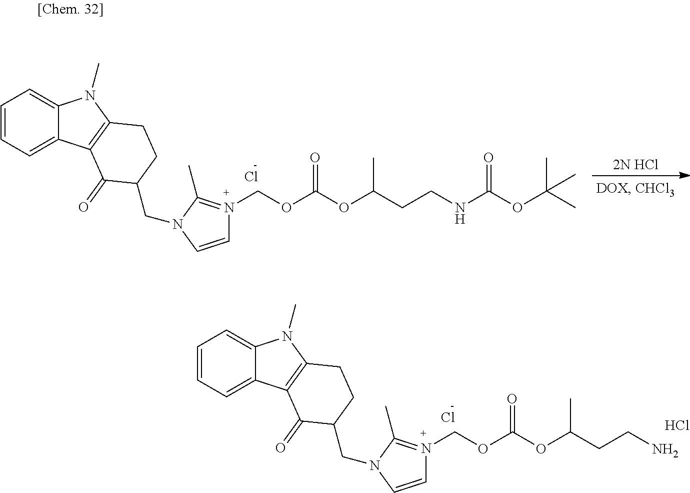

3 mL of 4 N hydrochloric acid/dioxane solution was added under cooling on ice to 3 mL of dioxane solution of 716 mg (1.25 mmol) of 3-[[[[3-[[(1,1-dimethylethoxy)carbonyl]amino]-1-methylpropoxy]carbonyl]oxy]methyl]-2-methyl-1-[(2,3,4,9-tetrahydro-9-methyl-4-oxo-1H-carbazol-3-yl)methyl]-1H-imidazolium chloride. The reaction solution was warmed to room temperature and left to stand still for 30 minutes. Thereafter, the solvent was distilled off under reduced pressure. Ethyl acetate was added to the residue and stirred and washed, Crystals were collected by filtration to obtain 640 mg (quantitative) of the title compound. ¹H-NMR (DMSO-d6, δ): 1.27 (3H, d, J=6 Hz), 1.88-2.02 (3H, m), 2.18-2.20 (1H, m), 2.78-2.82 (5H, m), 2.98-3.20 (3H, m), 3.75 (3H, s), 4.35 (1H, dd, J=14, 7 Hz), 4.72 (1H, dd, J=14, 7 Hz), 4.80-4.84 (1H, m), 6.14 (1H, d, J=12 Hz), 6.17 (1H, d, J=12 Hz), 7.22 (1H, t, J=7 Hz), 7.27 (1H, d, J=7 Hz), 7.56 (1H, d, J=7 Hz), 7.81 (1H, d, J=2 Hz), 7.85 (1H, d, J=2 Hz), 7.98 (1H, d, J=7 Hz), 8.15 (3H, br-s)

### Example 2

### [3-[[[(ondansetron)methoxy]carbonyl]oxy]butyl]amino-chondroitin sulfate conjugate

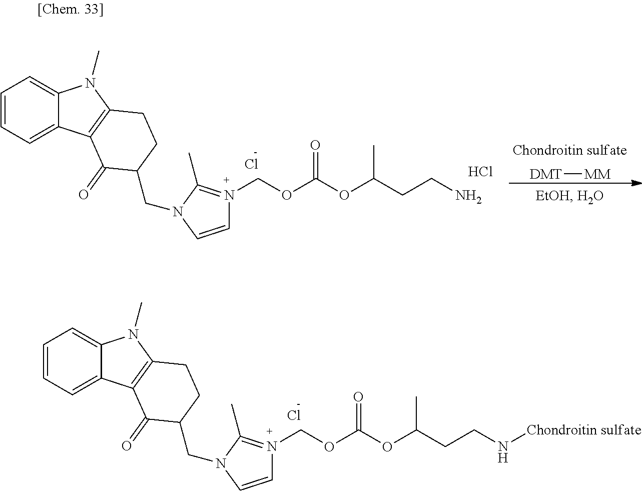

2 mL of ethanol was slowly added dropwise under stirring to 4.0 g (0.398 mmol) of 5% sodium Chondroitin sulfate aqueous solution. To the mixed liquid, a solution of 41 mg (0.080 mmol) of 3-[[[(3-amino-1-methylpropoxy)carbonyl]oxy]methyl]-2-methyl-1-[(2,3,4,9-tetrahydro-9-methyl-4-oxo-1H-carbazol-3-yl)methyl]-1H-imidazolium chloride hydrochloride in 1 mL of ethanol was added, a solution of 38 mg (0.136 mmol) of 4-(4,6-dimethoxy-1,3,5-triazin-2-yl)-4-methylmorpholinium chloride (DMT-MM) in 1 mL of ethanol was then added, 1 mL of ethanol and 1 mL of water were further added, and the resultant liquid was stirred at room temperature overnight. 100 μL of 20% sodium chloride aqueous solution was added to the reaction solution, and ethanol was further added dropwise (2 mL) until immediately before the reaction solution became cloudy. The reaction solution was added dropwise wider stirring to 12 mL of 93% ethanol, and 5 mL of ethanol was added to the mixed liquid and stirred for 1 hour. Precipitates were isolated using a centrifuge, washed twice with 90% ethanol, twice with ethanol, and twice with diethyl ether. The obtained precipitates were dried overnight using a vacuum pump to obtain 202 mg of the title compound. Based on values of integral in ¹H-NMR, the introduction ratio of ondansetron per whole disaccharide unit (glucuronic acid) of chondroitin sulfate was 13 mol %.

### Reference Example 7

### N-(3-hydroxy-4,4-dimethylpentyl)carbamic acid 1,1-dimethylethyl ester

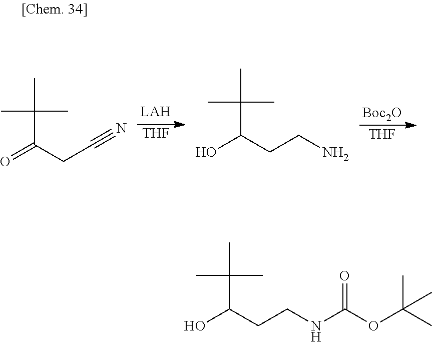

A tetrahydrofuran solution of 3.0 g (23.97 mmol) of 4,4-dimethyl-3-oxopentanenitrile was added dropwise under cooling on ice to a tetrahydrofuran solution of 2.09 g (55.13 mmol) of lithium aluminum hydride. The reaction solution was warmed to room temperature and stirred overnight, water and anhydrous magnesium sulfate were sequentially added to the reaction solution, and the resultant liquid was filtered through celite. The solvent was distilled off under reduced pressure to obtain 1.59 g of partially purified 1-amino-4,4-dimethyl-3-pentanol. 3.74 g (1.7.15 mmol) of Cert-butyl dicarbonate was added to a tetrahydrofuran (20 mL) solution of 1.50 g (1.43 mmol) of the partially purified 1-amino-4,4-dimethyl-3-pentanol and stirred overnight, and then the solvent was distilled off under reduced pressure. The residue was purified by silica gel column chromatography (11% ethyl acetate/hexane) to obtain 793 mg (30%) of the title compound.

¹H-NMR (CDCl₃, δ): 0.90 (9H, s), 1.45 (9H, s), 1.55-1.70 (2H, m), 2.45 (1H, br-s), 3.13-3.27 (2H, m), 3.45 (1H, m), 4.85 (1H, br-s)

### Reference Example 8

### Carbonic acid chloromethyl 1-[2-[[(1,1-dimethylethoxy)carbonyl]amino]ethyl]-2,2,-dimethylpropyl ester

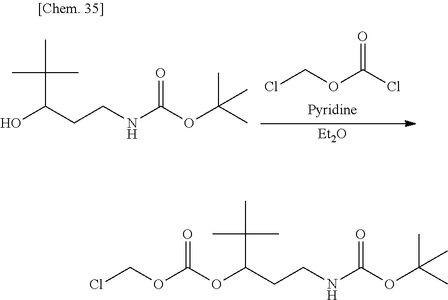

A diethyl ether solution (4 mL) of 526 mg (4.08 mmol) of chloromethyl chloroformate was added at −15° C. to a diethyl ether (10 mL) solution of 787 mg (3.04 mmol) of N-(3-hydroxy-4,4-dimethylpentyl)carbamic acid 1,1-dimethylethyl ester, and subsequently, a diethyl ether (4 mL) solution of 323 mg (4.08 mmol) of pyridine was added dropwise. After the reaction solution was was med to room temperature and stirred overnight, a diethyl ether (8 mL) solution of 1052 mg (8.16 mmol) of chloromethyl chloroformate and 646 mg (8.16 mmol) of pyridine was added dropwise again at −15° C., and the resultant liquid was stirred at room temperature overnight. Water was added to the reaction solution, and the resultant liquid was extracted with diethyl ether. The obtained organic layer was washed with saturated ammonium chloride aqueous solution, saturated sodium bicarbonate water, and saturated saline and dried over anhydrous magnesium sulfate, and the solvent was distilled off under reduced pressure. The residue was purified by silica gel column chromatography (1→10% ethyl acetate/hexane) to obtain 668 mg (61%) of the title compound.

¹H-NMR (CDCl₃, δ): 0.94 (9H, s), 1.44 (9H, s), 1.60-1.67 (1H, m), 1.89-1.93 (1H, m), 2.87-2.94 (1H, m), 3.36-3.38 (1H, m), 4.62 (1H, dd, J=11, 2 Hz), 4.90 (1H, br-s), 5.73-5.74 (2H, m)

### Reference Example 9

### 3-[[[[1-[2-[[(1,1-dimethylethoxy)carbonyl]amino]ethyl]-2,2-dimethylpropoxy]carbonyl]oxy]methyl]-2-methyl-1-[(2,3,4,9-tetrahydro-9-methyl-4-oxo-1H-carbazol-3-yl)methyl]-1H-imidazolium chloride

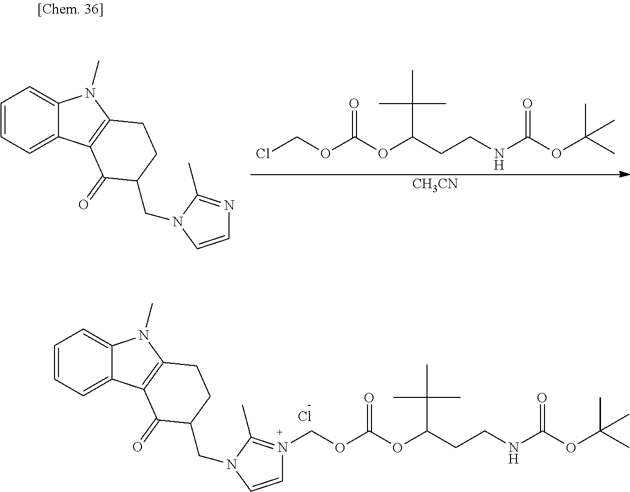

701 mg (2.39 mmol) of ondansetron was added at room temperature to an acetonitrile solution of 644 mg (1.99 mmol) of carbonic acid chloromethyl 1-[[2-[(1,1-dimethylethoxy)carbonyl]amino]ethyl]-2,2,-dimethylpropyl ester and stirred at 85° C. overnight. The reaction solution was condensed in a water bath set to 40° C. The residue was purified by silica gel column chromatography (8→20% methanol/chloroform) to obtain 806 mg (66%) of the title compound.

¹H-NMR (CDCl₃, δ): 0.89 (9H, s), 1.42 (9/2H, s), 1.43 (9/2H, s), 1.61-1.68 (1H, m), 1.83-2.09 (2H, m), 2.74-2.77 (1H, m), 2.91-3.35 (8H, m), 3.68 (3H, s), 4.54-4.58 (2H, m), 4.75-4.80 (2H, m), 6.15-6.23 (2H, m), 7.24-7.32 (3H, m), 7.47-7.48 (1H, m), 7.94-7.95 (1H, m), 8.09-8.10 (1H, m)

### Reference Example 10

### 3-[[[[1-(2-aminoethyl)-2,2-dimethylpropoxy]carbonyl]oxy]methyl]-2-methyl-1-[(2,3,4,9-tetrahydro-9-methyl-4-oxo-1H-carbazol-3-yl)methyl]-1H-imidazolium chloride hydrochloride

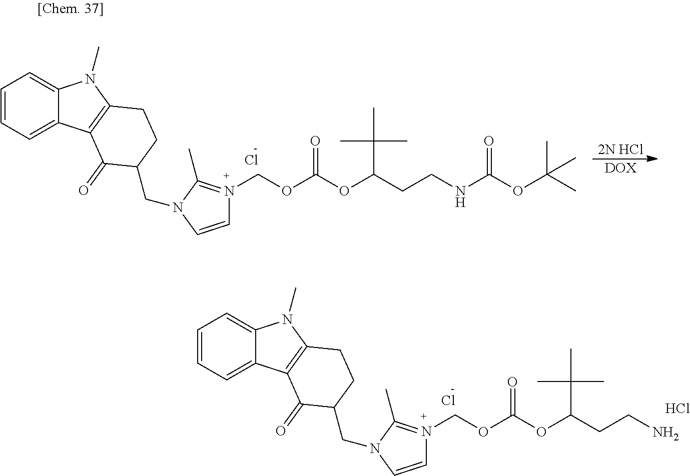

3 ml, of 4 N hydrochloric acid/dioxane solution was added under cooling on ice to 3 mL of dioxane solution of 800 mg (1.30 mmol) of 3-[[[[1-[2-[[(1,1-dimethylethoxy)carbonyl]amino]ethyl]-2,2-dimethylpropoxy]carbonyl]oxy]methyl]-2-methyl-1-[(2,3,4,9-tetrahydro-9-methyl-4-oxo-1H-carbazol-3-yl)methyl]-1H-imidazolium chloride. The reaction solution was warmed to room temperature and left to stand still for 30 minutes, Thereafter, the solvent was distilled off under reduced pressure. Ethyl acetate was added to the residue and stirred and washed. Crystals were collected by filtration to obtain 718 mg (quantitative) of the title compound.

¹H-NMR (DMSO-d6, δ): 0.86 (9/2H, s), 0.87 (9/2H, s), 1.75-2.17 (4H, m), 2.79 (5H, br-s), 2.99-3.19 (3H, m), 3.75 (3H, s), 4.35-4.40 (1H, m), 4.55-4.57 (1H, m), 4.73 (1H, dd, J=14, 7 Hz), 6.17 (1/2H, d, J=12 Hz), 6.18 (1/2H, d, J=12 Hz), 6.22 (1/2H, d, J=12 Hz), 6.23 (1/2H, d, J=12 Hz), 7.22 (1H, t, J=8 Hz), 7.27 (1H, t, J=8 Hz), 7.56 (1H, d, J=8 Hz), 7.82-7.87 (2H, m), 7.98 (1H, d, J=8 Hz), 8.12 (3H, br-s)

### Example 3

### [4,4-dimethyl-3-[[[(ondansetron)methoxy]carbonyl]oxy]pentyl]amino-chondroitin sulfate conjugate

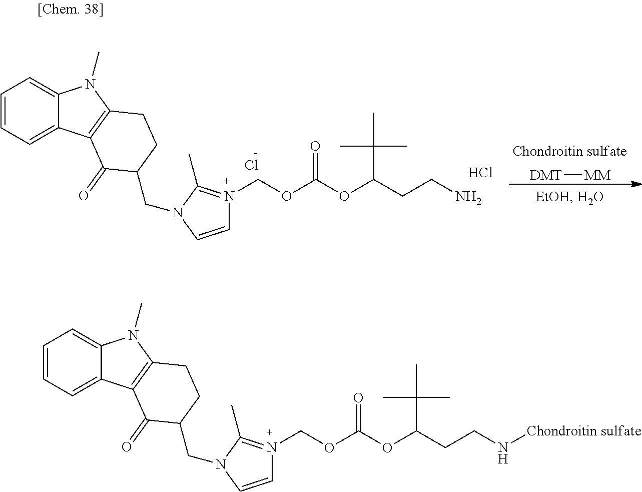

2 mL of ethanol was slowly added dropwise under stirring to 4.0 g (0.398 mmol) of 5% sodium chondroitin sulfate aqueous solution. To the mixed liquid, 44 mg (0.080 mmol) of 3-[[[[1-(2-aminoethyl)-2,2-dimethylpropoxy]carbonyl]oxy]methyl]-2-methyl-1-[(2,3,4,9-tetrahydro-9-methyl-4-oxo-1H-carbazol-3-yl)methyl]-1H-imidazolium chloride hydrochloride in 1 ML of ethanol was added, 38 mg (0.136 mmol) of 4-(4,6-dimethoxy-1,3,5-triazin-2-yl)-4-methylmorpholinium chloride (DMT-MM) in 1 mL of ethanol was then added, 1 mL of ethanol and 1 mL of water were further added, and the resultant liquid was stirred at room temperature overnight. 100 μL of 20% sodium chloride aqueous solution was added to the reaction solution, and ethanol was further added dropwise (2 mL) until immediately before the reaction solution became cloudy. The reaction solution was added dropwise under stirring to 12 mL of 93% ethanol, and 5 mL of ethanol was added to the mixed liquid and stirred for 1 hour. Precipitates were isolated using a centrifuge, washed twice with 90% ethanol, twice with ethanol, and twice with diethyl ether. The obtained precipitates were dried overnight using a vacuum pump to obtain 202 mg of the title compound. Based on values of integral in ¹H-NMR, the introduction ratio of ondansetron per whole disaccharide unit (glucuronic acid) of chondroitin sulfate was 10 mol %.

### Reference Example 11

### Carbonic acid chloromethyl 3-[[(1,1-dimethylethoxy)carbonyl]amino]-1,1-dimethylpropyl ester

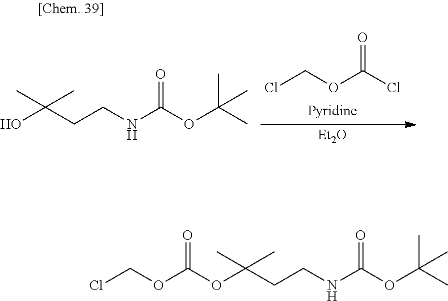

A diethyl ether solution (6 mL) of L16 g (8.99 mmol) of chloromethyl chloroformate was added at −15° C. to a diethyl ether (10 mL) solution of 457 mg (2.25 mmol) of N-(3-hydroxy-3-methylbutyl)carbamic acid 1,1-dimethylethyl ester, and subsequently, a diethyl ether (6 mL) solution of 711 mg (8.99 mmol) of pyridine was added dropwise. After the reaction solution was warmed to room temperature and stirred overnight, a diethyl ether solution of 2.32 g (18.00 mmol) of chloromethyl chloroformate and 1.42 g (18.00 mmol) of pyridine was added dropwise again at −15° C.; and the resultant liquid was stirred at room temperature for 8 hours. Water was added to the reaction solution, and the resultant liquid was extracted using diethyl ether. The obtained organic layer was washed with saturated ammonium chloride aqueous solution, saturated sodium bicarbonate water, and saturated saline and dried over anhydrous magnesium sulfate, and the solvent was distilled off under reduced pressure. The residue was purified by silica gel column chromatography (2→80% ethyl acetate/hexane) to obtain 472 mg (71%) of the title compound.

¹H-NMR (CDCl₃, δ): 1.44 (9H, s), 1.54 (6H, s), 2.00 (2H, t, J=8 Hz), 3.23-3.24 (2H, m), 4.58 (1H, br-s), 5.68 (2H, s)

### Reference Example 12

### 3-[[[[3-[[(1,1-dimethylethoxy)carbonyl]amino]-1,1-dimethylpropoxy]carbonyl]oxy]methyl]-2-methyl-1-[(2,3,4,9-tetrahydro-9-methyl-4-oxo-1H-carbazol-3-yl)methyl]-1H-imidazolium chloride

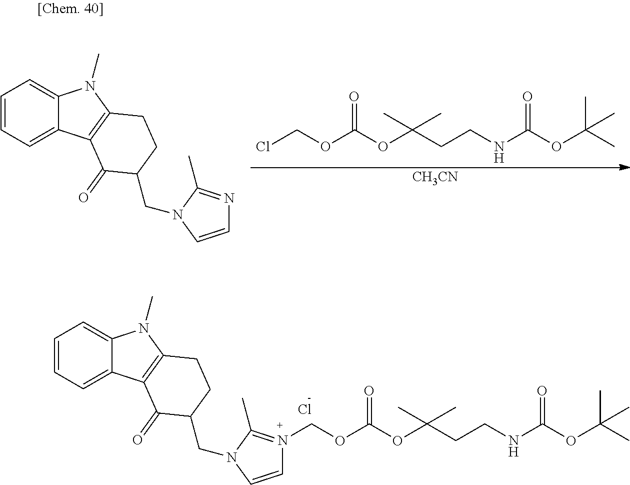

405 tug (1.38 mmol) of ondansetron was added at room temperature to an acetonitrile solution of 272 mg (0.93 mmol) of carbonic acid chloromethyl 3-[[(1,1-dimethylethoxy)carbonyl]amino]-1,1-dimethylpropyl ester and stirred at 85° C. overnight. The reaction solution was condensed in a water bath set to 40° C. The residue was purified by silica gel column chromatography (2→10% methanol/chloroform) to obtain 117 mg (22%) of the title compound.

¹H-NMR (CDCl₃, δ): 1.43 (9H, s), 1.50 (6H, s), 1.96-2.08 (3H, m), 2.81-2.84 (1H, m), 3.05 (3H, s), 3.12-3.36 (5H, m), 3.70 (3H, s), 4.59-4.65 (2H, m), 4.79 (1H, dd, J=14, 7 Hz), 6.06 (1H, d, J=12 Hz), 6.09 (1H, d, J=12 Hz), 7.26-7.32 (3H, m), 7.41 (1H, br-s), 7.96 (1H, br-s), 8.09-8.11 (1H, in.)

### Reference Example 13

### 3-[[[(3-amino-1,1-dimethylpropoxy)carbonyl]oxy]methyl]-2-methyl-1-[(2,3,4,9-tetrahydro-9-methyl-4-oxo-1H-carbazol-3-yl)methyl]-1H-imidazolium chloride hydrochloride

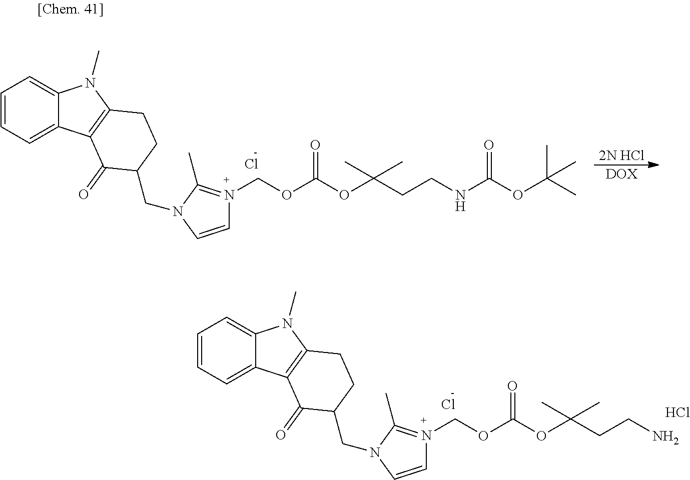

2 mL of 4 N hydrochloric acid/dioxane solution was added under cooling on ice to 2 mL of dioxane solution of 110 mg (0.19 mmol) of 3-[[[[3-[[(1,1-dimethylethoxy)carbonyl]amino]-1,1-dimethylpropoxy]carbonyl]oxy]methyl]-2-methyl-1-[(2,3,4,9-tetrahydro-9-methyl-4-oxo-1H-carbazol-3-yl)methyl]-1H-imidazolium chloride. The reaction solution was warmed to room temperature and left to stand still for 30 minutes. Thereafter, the solvent was distilled off under reduced pressure. Diisopropyl ether was added to the residue and stirred and washed. The obtained crystals were dissolved in water and washed with dichloromethane, and then the solution was freeze-dried to obtain 36 mg (35%) of the title compound.

¹H-NMR (DMSO-d6, δ): 1.47 (6H, s), 1.95-2.20 (4H, m), 2.77 (3H, s), 2.81-3.31 (5H, m), 3.75 (3H, s), 4.35 (1H, dd, J=14, 7 Hz), 4.72 (1H, dd, J=14, 7 Hz), 6.12 (2H, s), 7.20-7.28 (2H, m), 7.55-7.57 (1H, m), 7.81 (1H, d, J=3 Hz), 7.84 (1H, dd, J=3 Hz), 7.97-3.00 (1H, m), 8.12 (3H, br-s)

### Example 4

### [3-methyl-3-[[[(ondansetron)methoxy]carbonyl]oxy]butyl]amino-chondroitin sulfate conjugate

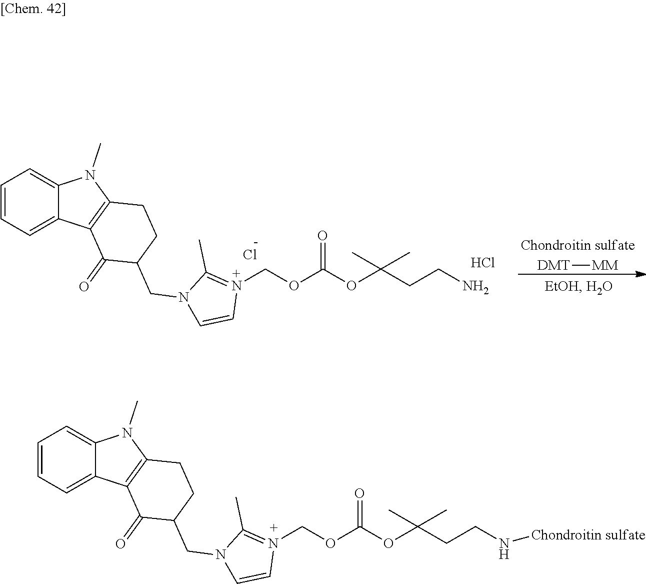

1 mL of ethanol was slowly added dropwise under stirring to 2.0 g (0.199 mmol) of 5% sodium chondroitin sulfate aqueous solution. To the mixed liquid, a solution of 21 mg (0.040 mmol) of 3-[[[(3-amino-1,1-dimethylpropoxy)carbonyl]oxy]methyl]-2-methyl-1-[(2,3,4,9-tetrahydro-9-methyl-4-oxo-1H-carbazol-3-yl)methyl]-1H-imidazolium chloride hydrochloride in 0.5 mL of ethanol was added, a solution of 19 mg (0.069 mmol) of 4-(4,6-dimethoxy-1,3,5-triazin-2-yl)-4-methylmorpholinium chloride (DMT-MM) in 0.5 mL of ethanol was then added. 0.5 mL of ethanol and 0.5 mL of water were further added, and the resultant liquid was stirred at room temperature overnight, 50 μL of 20% sodium chloride aqueous solution was added to the reaction solution, and ethanol was further added dropwise (1 mL) until immediately before the reaction solution became cloudy. The reaction solution was added dropwise under stirring to 6 mL of 93% ethanol, and 2.5 mL of ethanol was added to the mixed liquid and stirred for 1 hour. Precipitates were isolated using a centrifuge, washed twice with 90% ethanol, twice with ethanol, and twice with diethyl ether. The obtained precipitates were dried overnight using a vacuum pump to obtain 78 mg of the title compound. Based on values of integral in ¹H-NMR, the introduction ratio of ondansetron per whole disaccharide unit (glucuronic acid) of chondroitin sulfate was 5 mol %.

### Reference Example 14

### Carbonic acid chloromethyl 3-[[(1,1-dimethylethoxy)carbonyl]amino]-1-phenylpropyl ester

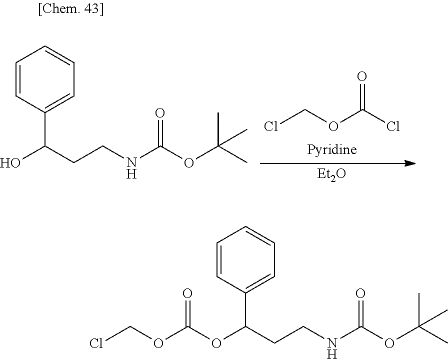

A diethyl ether solution (4 rut) of 589 mg (4.57 mmol) of chloromethyl chloroformate was added at −15° C. to a diethyl ether (10 mL) solution of 574 mg (2.28 mmol) of N-(3-hydroxy-3-phenylpropyl)carbamic acid 1,1-dimethylethyl ester, subsequently, a diethyl ether (4 mL) solution of 451 mg (5.70 mmol) of pyridine was added dropwise, and 1.0 mL of diethyl ether was further added. The reaction solution was warmed to room temperature and stirred overnight, water was then added to the reaction solution, and the resultant liquid was extracted with diethyl ether. The obtained organic layer was washed with saturated ammonium chloride aqueous solution, saturated sodium bicarbonate water, and saturated saline and dried over anhydrous magnesium sulfate, and the solvent was distilled off under reduced pressure to obtain 779 mg (99%) of the title compound.

¹H-NMR (CDCl₃, δ): 1.44 (9H, s), 2.05-2.21 (2H, m), 3.14-3.24 (2H, m), 4.67 (1H, br-s), 5.66-5.77 (3H, m), 7.31-7.39 (5H, m)

### Reference Example 15

### 3-[[[[3-[[(1,1-dimethylethoxy)carbonyl]amino]-1-phenylpropoxy]carbonyl]oxy]methyl]-2-methyl-1-[(2,3,4,9-tetrahydro-9-methyl-4-oxo-1H-carbazol-3-yl)methyl]-1H-imidazolium chloride

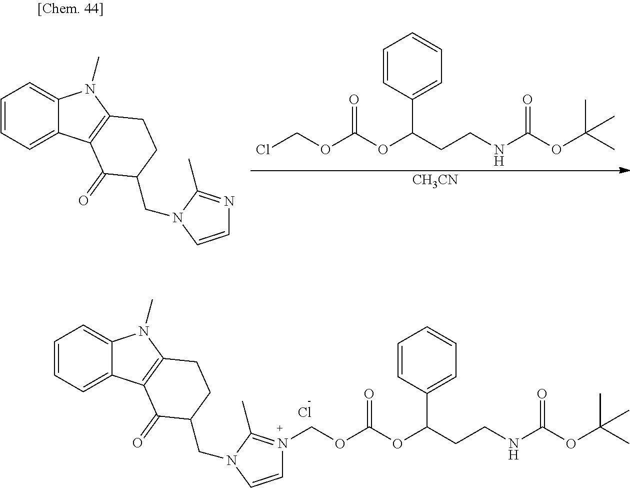

536 mg (1.83 mmol) of ondansetron was added at room temperature to an acetonitrile solution of 445 mg (L29 mmol) of carbonic acid chloromethyl 3-[[(1,1-dimethylethoxy)carbonyl]amino]-1-phenylpropyl ester and stirred at 85° C. overnight. The reaction solution was condensed in a water bath set to 40° C. The residue was purified by silica gel column chromatography (10-415% methanol/chloroform) to obtain 271 mg (33%) of the title compound,

¹H-NMR (CDCl₃, δ): 1.41 (9/2H, s), 1.42 (9/2H, s), 1.96-2.20 (3H, m), 2.78-2.79 (1H, m), 3.01-3.35 (8H, m), 3.69 (3H, s), 4.49-4.55 (1H, m), 4.74-4.80 (2H, m), 5.59-5.63 (1H, m), 6.08 (1/2H, d, J=12 Hz), 6.09 (1/2H, d, J=12 Hz), 6.13 (1/2H, d, J=12 Hz), 6.14 (1/2H, d, J=1.2 Hz), 7.25-7.37 (9H, m), 7.82 (1/2H, d, J=2 Hz), 7.83 (1/2H, d, J=2 Hz), 8.10-8.12 (1H, m)

### Reference Example 16

### 3-[[[(3-amino-1-phenylpropoxy)carbonyl]oxy]methyl]-2-methyl-1-[(2,3,4,9-tetrahydro-9-methyl-4-oxo-1H-carbazol-3-yl)methyl]-1H-imidazolium chloride hydrochloride

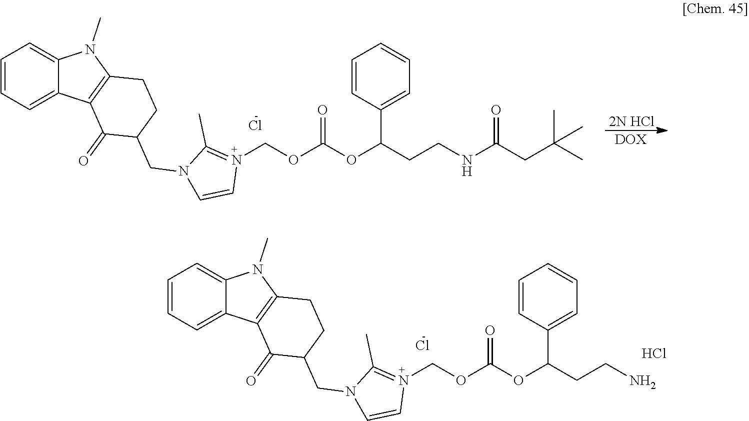

2 mL of 4 N hydrochloric acid/dioxane solution was added under cooling on ice to 2 mL of dioxane solution of 141 rug (0.22 mmol) of 3-[[[[3-[[(1,1-dimeth ylethoxy)carbonyl]amino]-1-phenylpropoxy]carbonyl]oxy]methyl]-2-methyl-1-[(2,3,4,9-tetrahydro-9-methyl-4-oxo-1H-carbazol-3-yl)methyl]-1H-imidazolium chloride. The reaction solution was warmed to room temperature and left to stand still for 1 hour. Thereafter, the solvent was distilled off under reduced pressure. Diisopropyl ether was added to the residue and stirred and washed. Crystals were collected by filtration to obtain 102 mg (81%) of the title compound.

¹H-NMR (DMSO-d6, δ): 1.96 (1H, qd, J=12, 6 Hz), 2.07-2.29 (3H, m), 2.75-2.83 (5H, m), 2.97-3.19 (3H, m), 3.75 (3H, s), 4.33 (1H, dd, J=14, 7 Hz), 4.77 (1H, dd, J=14, 7 Hz), 5.72-5.75 (1H, m), 6.12 (1/2H, d, J=12 Hz), 6.13 (1/2H, d, J=12 Hz), 6.16 (1/2H, d, J=12 Hz), 6.17 (1/2H, d, J=12 Hz), 7.21-7.28 (2H, m), 7.35-7.42 (5H, m), 7.56-7.58 (1H, m), 7.79 (1H, d, J=2 Hz), 7.82-7.83 (1H, m), 7.99 (1H, d, J=8 Hz), 8.22 (3H, br-s)

### Example 5

### [3-[[[(ondansetron)methoxy]carbonyl]oxy]-3-phenylpropyl]amino-chondroitin sulfate conjugate

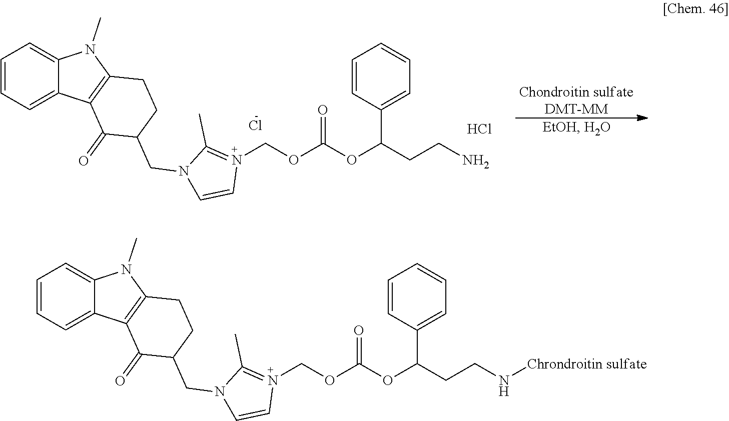

2 mL of ethanol was slowly added dropwise under stirring to 4.0 g (0.398 mmol) of 5% sodium chondroitin sulfate aqueous solution. To the mixed liquid, a solution of 46 mg (0,080 mmol) of 3-[[[(3-amino-1-phenylpropoxy)carbonyl]oxy]methyl]-2-methyl-1-[(2,3,4,9-tetrahydro-9-methyl-4-oxo-1H-carbazol-3-yl)methyl]-1H-imidazolium chloride hydrochloride in 1 mL of ethanol was added, a solution of 38 mg (0.136 mmol) of 4-(4,6-dimethoxy-1,3,5-triazin-2-yl)-4-methylmorpholinium chloride (DMT-MM) in 1 mL of ethanol was then added, 1 mL of ethanol and 1 mL of water were further added, and the resultant liquid was stirred at room temperature overnight. 100 μL of 20% sodium chloride aqueous solution was added to the reaction solution, and ethanol was further added dropwise (2 mL) until immediately before the reaction solution became cloudy. The reaction solution was added dropwise under stirring to 12 mL of 93% ethanol, and 5 mL of ethanol was added to the mixed liquid and stirred for 1 hour. Precipitates were isolated using a centrifuge, washed twice with 90% ethanol, twice with ethanol, and twice with diethyl ether. The obtained precipitates were dried overnight using a vacuum pump to obtain 198 mg of the title compound. Based on values of integral in ¹H-NMR, the introduction ratio of ondansetron per whole disaccharide unit (glucuronic acid) of chondroitin sulfate was 11 mol %.

### Reference Example 17

### Carbonic acid chloromethyl 1-cyclohexyl-3-[[(1,1-dimethylethoxy)carbonyl]amino]propyl ester

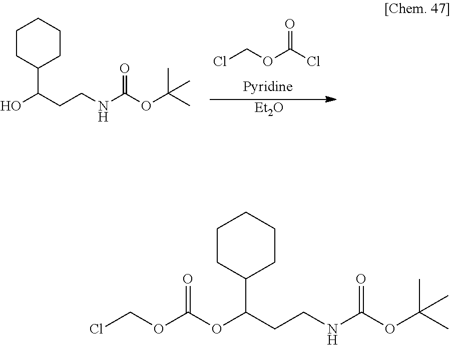

A diethyl ether solution (5 mL) of 462 mg (3.58 mmol) of chloromethyl chloroformate was added at −15° C. to a diethyl ether (30 mL) solution of 460 mg (1.79 mmol) of N-(3-cyclohexyl-3-hydroxypropyl)carbamic acid 1,1-dimethylethyl ester, and subsequently, a diethyl ether (7 mL) solution of 354 mg (4.48 mmol) of pyridine was added dropwise. The reaction solution was warmed to room temperature and stirred overnight, water was then added to the reaction solution, and the resultant liquid was extracted with diethyl ether. The obtained organic layer was washed with saturated ammonium chloride aqueous solution, saturated sodium bicarbonate water, and saturated saline and dried over anhydrous magnesium sulfate, and the solvent was distilled off under reduced pressure. The residue was purified by silica gel column chromatography (2→10% ethyl acetate/hexane) to obtain 485 mg (79%) of the title compound.

¹H-NMR (CDCl₃, δ): 1.00-1.27 (5H, m), 1.44 (9H, s), 1.54-1.77 (7H, m), 1.85-1.86 (1H, m), 2.96-3.03 (1H, m), 3.30-3.32 (1H, m), 4.67-4.70 (1H, m), 4.79 (1H, br-s). 5.72 (2H, s)

### Reference Example 18

### 3-[[[[1-cyclohexyl-3-[[(1,1-dimethylethoxy)carbonyl]amino]propoxy]carbonyl]oxy]methyl]-2-methyl-1-[(2,3,4,9-tetrahydro-9-methyl-4-oxo-1H-carbazol-3-yl)methyl]-1H-imidazolium chloride

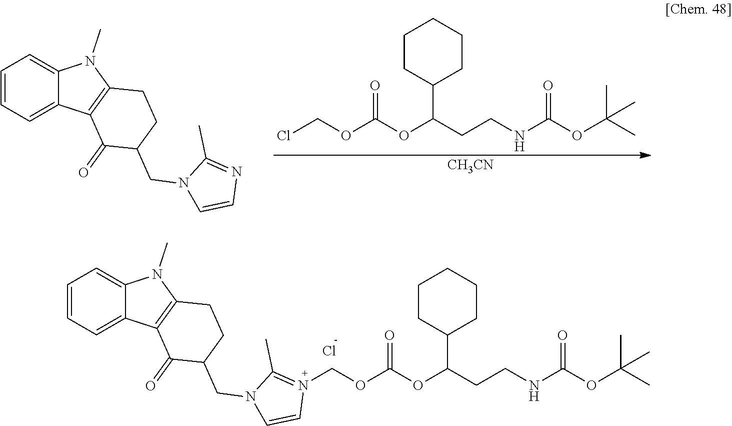

405 mg (1.38 mmol) of ondansetron was added at room temperature to an acetonitrile solution of 479 mg (1.37 mmol) of carbonic acid chloromethyl 1-cyclohexyl-3-[[(1,1-dimethylethoxy)carbonyl]amino]propyl ester and stirred at 85° C. overnight. The reaction solution was filtered, and then the filtrate was condensed in a water bath set to 40° C. The residue was purified by silica gel column chromatography (10% methanol/chloroform) to obtain 517 mg (59%) of the title compound.

¹H-NMR (CDCl₃, δ): 0.96-1.29 (5H, m), 1.41 (9H, s), 1.53-1.93 (8H, m), 2.05 (1H, qd, J=13, 6 Hz), 2.77-2.79 (1H, m), 3.02-3.37 (8H, m), 3.69 (3H, s), 4.56-4.64 (2H, m), 4.75-4.81 (2H, m), 6.15 (1/2H, d, J=1.2 Hz), 6.16 (1/2H, d, J=12 Hz), 6.19 (1/2H, d, J=12 Hz), 6.20 (1/2H, d, J=12 Hz), 7.24-7.32 (3H, m), 7.46 (1/2H, d, J=2 Hz), 7.47 (1/2H, d, J=2 Hz), 7.94 (1/2H, d, J=2 Hz), 7.97 (1/2H, br-s), 8.09-8.11 (1H, m)

### Reference Example 19

### 3-[[[(3-amino-1-cyclohexyl propoxy)carbonyl]oxy]methyl]-2-methyl-1-[(2,3,4,9-tetrahydro-9-methyl-4-oxo-1H-carbazol-3-yl)methyl]-1H-imidazolium chloride hydrochloride

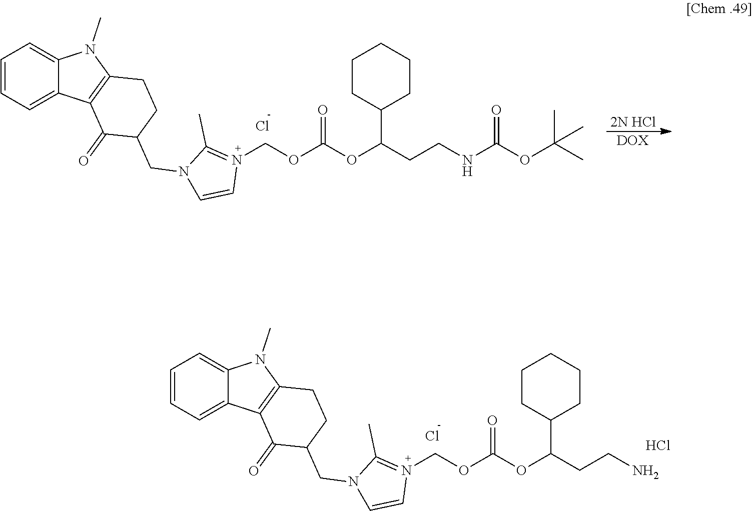

3 mL of 4 N hydrochloric acid/dioxane solution was added under cooling on ice to 3 mL of dioxane solution of 503 mg (0.78 mmol) of 3-[[[[1-cyclohexyl-3-[[(1,1-dimethylethoxy)carbonyl]amino]propoxy]carbonyl]oxy]methyl]-2-methyl-1-[(2,3,4,9-tetrahydro-9-methyl-4-oxo-1H-carbazol-3-yl)methyl]-1H-imidazolium chloride. The reaction solution was warmed to room temperature and left to stand still for 1 hour. Thereafter, the solvent was distilled off under reduced pressure. Ethyl acetate was added to the residue and stirred and washed. Crystals were collected by filtration to obtain 444 mg (98%) of the title compound.

¹H-NMR (DMSO-d6, δ): 0.85-1.28 (6H, m) 1.55-1.69 (5H, m), 1.84-2.02 (3H, m), 2.16-2.19 (1H, m), 2.75-2.82 (5H, m), 2.98-3.20 (3H, m), 3.75 (3H, s), 4.36 (1/2H, dd, J=14, 7 Hz), 4.37 (1/2H, dd, J=14, 7 Hz), 4.61-4.62 (1H, m), 4.73 (1H, dd, J=14, 7 Hz), 6.16 (1H, d, J=13 Hz), 6.18 (1H, d, J=13 Hz), 7.20-7.28 (2H, m), 7.56 (1H, d, J=8 Hz), 7.82 (1H, d, J=2 Hz), 7.86 (1/2H, d, J=2 Hz), 7.87 (1/2H, d, J=2 Hz), 7.98 (1H, d, J=8 Hz), 8.12 (3H, br-s)

### Example 6

### [3-cyclohexyl-3-[[[(ondansetron)methoxy]carbonyl]oxy]propyl]amino-chondroitin sulfate conjugate

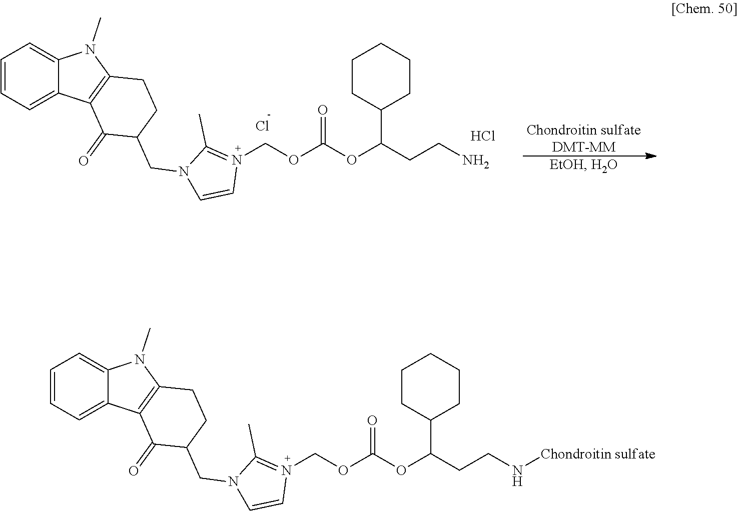

2 mL of ethanol was slowly added dropwise under stirring to 4.0 g (0.398 mmol) of 5% sodium chondroitin sulfate aqueous solution. To the mixed liquid, a solution of 46 mg (0.080 mmol) of 3-[[[(3-amino-1-cyclohexyl propoxy)carbonyl]oxy]methyl]-2-methyl-1-[(2,3,4,9-tetrahydro-9-methyl-4-oxo-1H-carbazol-3-yl)methyl]-1H-imidazolium chloride hydrochloride in 1 mL of ethanol was added, a solution of 38 mg (0.136 mmol) of 4-(4,6-dimethoxy-1,3,5-triazin-2-yl)-4-methylmorpholinium chloride (DMT-MM) in 1 mL of ethanol was then added, 1 mL of ethanol and 1 mL of water were further added, and the resultant liquid was stirred at room temperature overnight. 100 μL of 20% sodium chloride aqueous solution was added to the reaction solution, and ethanol was further added dropwise (2 mL) until immediately before the reaction solution became cloudy. The reaction solution was added dropwise under stirring to 12 mL of 93% ethanol, and 5 mL of ethanol was added to the mixed liquid and stirred for 1 hour. Precipitates were isolated using a centrifuge, washed twice with 90% ethanol, twice with ethanol, and twice with diethyl ether. The obtained precipitates were dried overnight using a vacuum pump to obtain 192 mg of the title compound. Based on values of integral in ¹H-NMR, the introduction ratio of ondansetron per whole disaccharide unit (glucuronic acid) of chondroitin sulfate was 10 mol %.

### Reference Example 20

### N-[2-[[(1,1-dimethylethoxy)carbonyl]amino]ethyl]carbamic acid chloromethyl ester

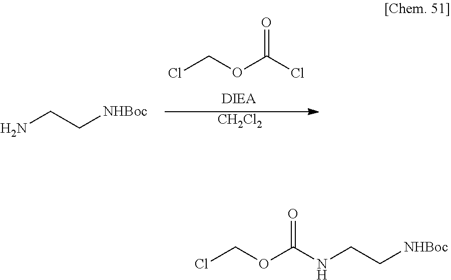

A methylene chloride solution of 0.666 mL (7.49 mmol) of chloromethyl chloroformate was added dropwise under cooling on ice to a methylene chloride solution of 1.00 g (6.24 mmol) of N-[(1,1-dimethylethoxy)carbonyl]-1,2-ethylenediamine and 1.21 mL (9.36 mmol) of N,N-diisopropylethylamine. After 15 minutes, water was then added to the reaction solution, and the resultant liquid was extracted with diethyl ether. The obtained organic layer was washed with 5% potassium hydrogensulfate aqueous solution, saturated sodium bicarbonate water, and saturated saline and dried over anhydrous magnesium sulfate, and the solvent was distilled off under reduced pressure. The residue was recrystallized by a hexane-chloroform mixed solvent to obtain 1.27 g (80%) of the title compound.

¹H-NMR (CDCl₃, δ): 1.44 (9H, s), 3.28-3.30 (2H, m), 3.33-3.36 (2H, m), 4.81 (1H, br-s), 5.50 (1H, br-s), 5.74 (2H, s)

### Reference Example 21

### 3-[[[[[2-[[(1,1-dimethylethoxy)carbonyl]amino]ethyl]amino]carbonyl]oxy]methyl]-2-methyl-1-[(2,3,4,9-tetrahydro-9-methyl-4-oxo-1H-carbazol-3-yl)methyl]-1H-imidazolium chloride

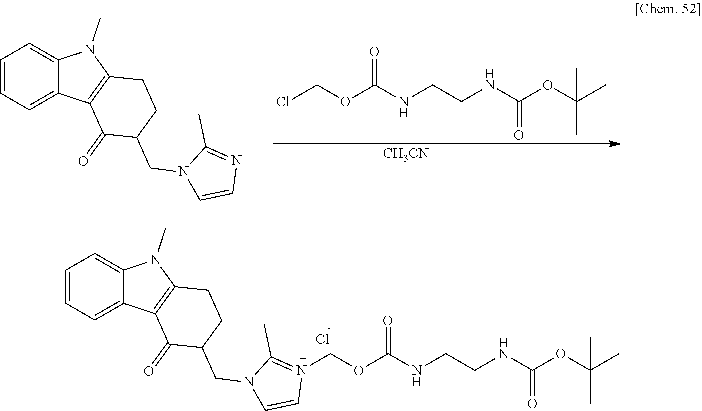

400 mg (L36 mmol) of ondansetron was added at room temperature to an acetonitrile solution of 342 mw. (1.35 mmol) of N-[2-[[(1,1-dimethylethoxy)carbonyl]amino]ethyl]carbamic acid chi ester and stirred at 85′C overnight. The reaction solution was filtered, and then the filtrate was condensed in a water bath set to 40° C. The residue was purified by silica gel column chromatography (10→20% methanol/chloroform) to obtain 365 mg (50%) of the title compound.

¹H-NMR (CDCl₃, δ): 1.40 (9H, s), 1.98-2.07 (1H, m), 2.63-2.65 (1H, m), 3.04 (3H, s), 3.13-3.26 (7H, m), 3.68 (3H, s), 4.52-4.54 (1H, m), 4.72 (1H, dd, J=14, 7 Hz), 5.67 (1H, br-s), 6.10 (2H, br-s), 7.25-7.31 (4H, m), 7.52 (1H, d, J=2 Hz), 7.78 (1H, br-s), 8.10-8.12 (1H, m)

### Reference Example 22

### 3-[[[[(2-aminoethyl)amino]carbonyl]oxy]methyl]-2-methyl-1-[(2,3,4,9-tetrahydro-9-methyl-4-oxo-1H-carbazol-3-yl)methyl]-1H-imidazolium chloride hydrochloride

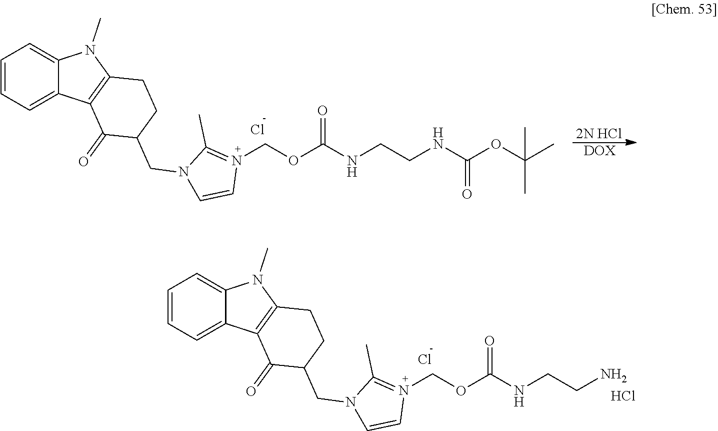

3 mL of 4 N hydrochloric acid/dioxane solution was added under cooling on ice to 3 mL of dioxane solution of 361 mg (0.66 mmol) of 3-[[[[[2-[[(1,1-dimethylethoxy)carbonyl]amino]ethyl]amino]carbonyl]oxy]methyl]-2-methyl-1-[(2,3,4,9-tetrahydro-9-methyl-4-oxo-1H-carbazol-3-yl)methyl]-1H-imidazolium chloride. The reaction solution was warmed to room temperature and left to stand still for 1 hour. Thereafter, the solvent was distilled off under reduced pressure. Ethyl acetate was added to the residue and stirred and washed, Crystals were collected by filtration to obtain 335 tug (quantitative) of the title compound.

¹H-NMR (DMSO-d6, δ): 1.94-2.02 (1H, m), 2.16-2.19 (1H, m), 2.79 (3H, s), 2.85-2.87 (2H, m), 2.98-3.30 (5H, m), 3.75 (3H, s), 4.34 (1H, dd, J=14, 7 Hz), 4.72 (1H, dd, J=14, 7 Hz), 6.06 (2H, s), 7.22 (1H, t, J=7 Hz), 7.27 (1H, t, J=7 Hz), 7.56 (1H, d, J=7 Hz), 7.78 (2H, s), 7.96 (1H, t, J=6 Hz), 7.99 (1H, d, J=7 Hz), 8.22 (3H, br-s)

### Example 7

### [2-[[[(ondansetron)methoxy]carbonyl]amino]ethyl]amino-chondroitin sulfate conjugate

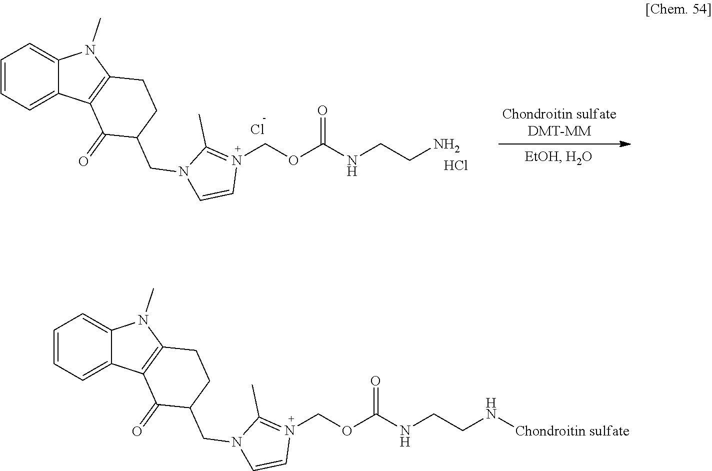

2 mL of ethanol was slowly added dropwise under stirring to 4.0 g (0.398 mmol) of 5% sodium chondroitin sulfate aqueous solution. To the mixed liquid, a solution of 39 mg (0.080 mmol) of 3-[[[[(2-aminoethyl)amino]carbonyl]oxy]methyl]-2-methyl-1-[(2,3,4,9-tetrahydro-9-methyl-4-oxo-1H-carbazol-3-yl)methyl]-1H-imidazolium chloride hydrochloride in 1 mL of ethanol was added, a solution of 38 mg (0.136 mmol) of 4-(4,6-dimethoxy-1,3,5-triazin-2-yl)-4-methylmorpholinium chloride (DMT-MM) in 1 mL of ethanol was then added, 1 mL of ethanol and 1 mL of water were further added, and the resultant liquid was stirred at room temperature overnight, 100 μL of 20% sodium chloride aqueous solution was added to the reaction solution, and ethanol was further added dropwise (2 mL) until immediately before the reaction solution became cloudy. The reaction solution was added dropwise under stirring to 12 mL of 93% ethanol, and 5 ad, of ethanol was added to the mixed liquid and stirred for 1 hour. Precipitates were isolated using a centrifuge, washed twice with 90% ethanol, twice with ethanol, and twice with diethyl ether. The obtained precipitates were dried overnight using a vacuum pump to obtain 193 mg of the title compound. Based on values of integral in ¹H-NMR, the introduction ratio of ondansetron per whole disaccharide unit (glucuronic acid) of chondroitin sulfate was 18 mol %,

### Reference Example 23

### N-[2-[[(1,1-dimethylethoxy)carbonyl]amino]ethyl]-N-(1-methylethyl)carbamic acid 1-chloro-2-methylpropyl ester

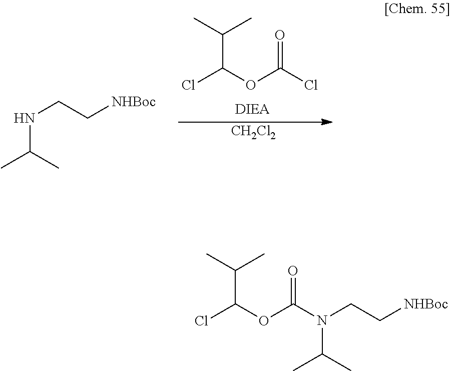

A methylene chloride solution of 0.434 mL (2.97 mmol) of 1-chloro-2-methylpropyl chloroformate was added dropwise under cooling on ice to a methylene chloride solution of 600 mg (2.97 mmol) of N-([(1,1-dimethylethoxy)carbonyl])-N′-(2-methylethyl)-ethylenediamine and 0.620 mL (3.56 mmol) of N,N-diisopropylethylamine. After 3 hours, water was then added to the reaction solution, and the resultant liquid was extracted with diethyl ether. The obtained organic layer was washed with 5% potassium hydrogensulfate aqueous solution, saturated sodium bicarbonate water, and saturated saline and dried over anhydrous magnesium sulfate, and the solvent was distilled off under reduced pressure. The residue was purified by silica gel column chromatography to obtain 989 mg (99%) of the title compound.

¹H-NMR (CDCl₃, δ): 1.07 (3H, d, J=7 Hz), 1.08 (3H, d, J=7 Hz), 1.20 (6H, d, J=7 Hz), 1.44 (9H, s), 2.17-2.23 (1H, m), 3.26-3.32 (4H, m), 4.13 (1/2H, br-s), 4.29 (1/2H, br-s), 4.73 (1/2H, br-s), 4.96 (1/2H, br-s), 6.37-6.38 (1H, m)

### Reference Example 24

### 3-[1-[[[[2-[[(1,1-dimethylethoxy)carbonyl]amino]ethyl](1-methylethyl)amino]carbonyl]carbonyl]oxy]-2-methylpropyl]-2-methyl-1-[(2,3,4,9-tetrahydro-9-methyl-4-oxo-1H-carbazol-3-yl)methyl]-1H-imidazolium chloride

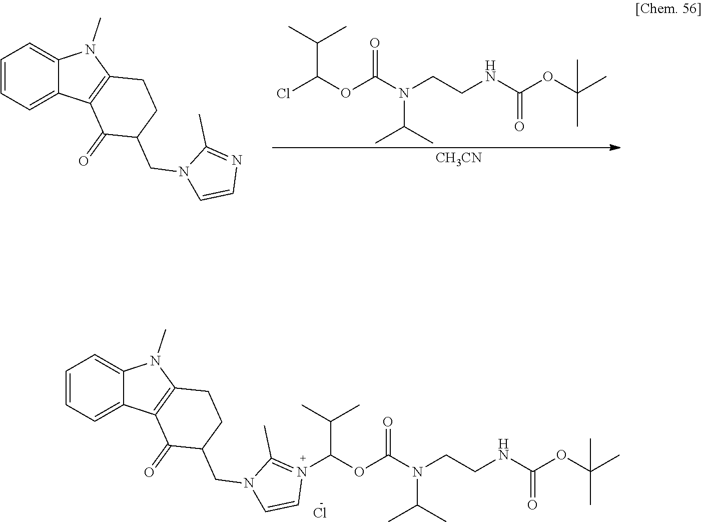

362 mg (1.23 mmol) of ondansetron was added at room temperature to an acetonitrile solution of 412 mg (1.22 mmol) of N-[2-[[(1,1-dimethylethoxy)carbonyl]amino]ethyl]-N-(1-methylethyl)carbamic acid 1-chloro-2-methylpropyl ester and stirred at 85° C. overnight. The reaction solution was condensed in a water bath set to 40° C. The residue was purified by silica gel column chromatography (10% methanol/chloroform) to obtain 97 mg (12%) of the title compound.

¹H-NMR (CDCl₃, δ): 0.77-0.94 (4H, m), 1.14-1.21 (8H, m), 1.42 (9/2H, s), 1.44 (9/2H, s), 2.10-2.12 (1H, m), 2.36-2.40 (1/2H, m), 2.62-2.63 (1/2H, m), 2.89-3.06 (4H, m), 3.15-3.49 (7H, m), 3.72 (3H, s), 4.13-4.20 (1H, m), 4.53-4.67 (1H, m), 4.76-4.96 (3/2H, m), 5.22 (1/4H, br-s), 5.37 (1/4H, br-s), 6.12-6.13 (1H, m), 7.26-7.32 (4H, m), 8.00-8.09 (3/2H, m), 8.23-8.30 (1/2H, m)

### Reference Example 25

### 3-[1-[[[(2-amino ethyl)(1-methylethyl)amino]carbonyl]oxy]-2-methylpropyl]-2-methyl-1-[(2,3,4,9-tetrahydro-9-methyl-4-oxo-1H-carbazol-3-yl)methyl]-1H-imidazolium hydrochloride

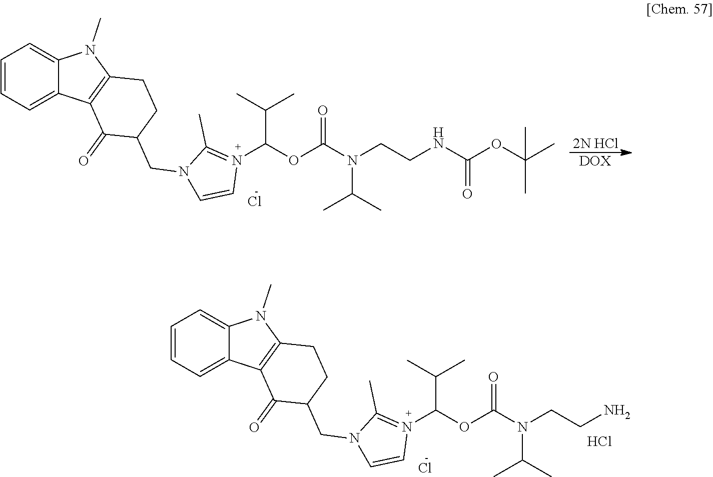

1 mL of 4 N hydrochloric acid/dioxane solution was added under cool on ice to 1 mL of dioxane solution of 97 mg (0.15 mmol) of 3-[1-[[[[2-[[(1,1-dimethylethoxy)carbonyl]amino]ethyl](1-methylethyl)amino]carbonyl]oxy]-2-methylpropyl]-2-methyl-1-[(2,3,4,9-tetrahydro-9-methyl-4-oxo-1H-carbazol-3-yl)methyl]-1H-imidazolium chloride. The reaction solution was warmed to room temperature and left to stand still for 1 hour. Thereafter, the solvent was distilled off under reduced pressure. Ethyl acetate was added to the residue and stirred and washed. Crystals were collected by filtration to obtain 79 mg (23%) of the title compound.

¹H-NMR (DMSO-d6, δ): 0.74-0.80 (3H, m), 1.05-1.19 (9H, m) 1.99-2.10 (2H, 2.63-2.88 (4H, m), 3.00-3.51 (7H, m), 3.75 (3H, s), 4.01-4.28 (1H, m), 4.32-4.41 (1H, m), 4.62-4.74 (1H, m), 6.31-6.37 (1H, m), 7.22 (1H, t, J=7 Hz), 7.27 (1H, t, J=7 Hz), 7.56 (3/2H, d, J=7 Hz), 7.86-8.00 (5/2H, m), 8.17 (1H, br-s), 8.32-8.34 (2H, m)

### Example 8

### [2-[[[2-methyl-1-(ondansetron)propoxy]carbonyl](1-methylethyl)amino]ethyl]amino-chondroitin sulfate conjugate

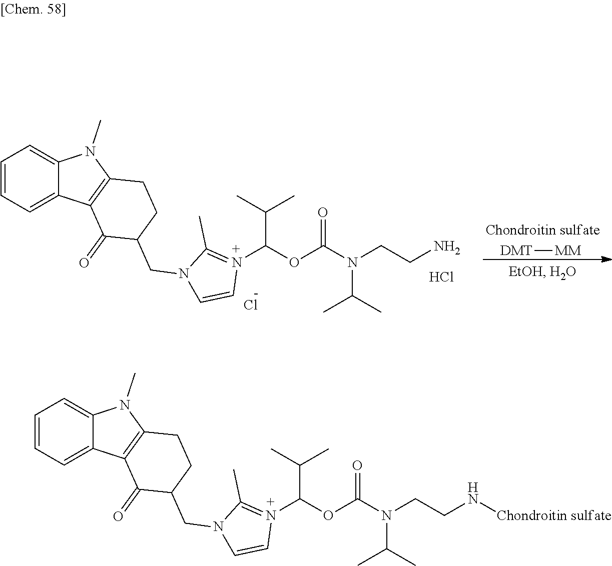

2 mL of ethanol was slowly added dropwise under stirring to 4.0 g (0.398 mmol) of 5% sodium chondroitin sulfate aqueous solution. To the mixed liquid, a solution of 45 mg (0.080 mmol) of 3-[1-[[[(2-aminoethyl)(1-methylethyl)amino]carbonyl]oxy]-2-methylpropyl]-2-methyl-1-[(2,3,4,9-tetrahydro-9-methyl-4-oxo-1H-carbazol-3-yl)methyl]-1H-imidazolium hydrochloride in 1 mL of ethanol was added, a solution of 38 mg (0.136 mmol) of 4-(4,6-dimethoxy-1,3,5-triazin-2-yl)-4-methylmorpholinium chloride (DMT-MM) in 1 mL of ethanol was then added, 1 mL of ethanol and 1 mL of water were further added, and the resultant liquid was stirred at room temperature overnight. 100 μL of 20% sodium chloride aqueous solution was added to the reaction solution, and ethanol was further added dropwise (3 mL) until immediately before the reaction solution became cloudy. The reaction solution was added dropwise under stirring to 12 mL of 93% ethanol, and 4 mL of ethanol was added to the mixed liquid and stirred for 1 hour. Precipitates were isolated using a centrifuge, washed twice with 90% ethanol, twice with ethanol, and twice with diethyl ether. The obtained precipitates were dried overnight using a vacuum pump to obtain 209 mg of the title compound. Based on values of integral in ¹H-NMR, the introduction ratio of ondansetron per whole disaccharide unit (glucuronic acid) of chondroitin sulfate was 18 mol %.

### Reference Example 26

### (3R)-3-[[(1,1-dimethylethoxy)carbonyl]amino]-1-piperidine carboxylic acid chloromethyl ester

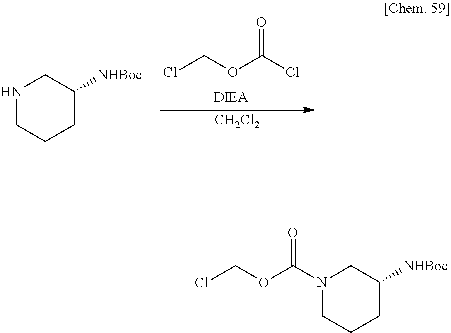

A methylene chloride solution of 386 mg (3.00 mmol) of chloromethyl chloroformate was added dropwise under cooling on ice to a methylene chloride solution of 500 mg (2.50 mmol) of (3R)-3-[[(1,1-dimethylethoxy)carbonyl]amino]piperidine and 0.652 mL (3.75 mmol) of N,N-diisopropylethylamine. After 15 minutes, water was then added to the reaction solution, and the resultant liquid was extracted with diethyl ether. The obtained organic layer was washed with 5% potassium hydrogensulfate aqueous solution, saturated sodium bicarbonate water, and saturated saline and dried over anhydrous magnesium sulfate, and the solvent was distilled off under reduced pressure. The residue was purified by silica gel column chromatography to obtain 630 mg (quantitative) of the title compound.

¹H-NMR (CDCl₃, δ): 1.45-1.55 (11H, m), 1.69-1.73 (1H, m), 1.87-1.91 (1H, m), 3.22-3.35 (2H, m), 3.57-3.77 (3H, m), 4.54 (1H, br-s), 5.72-5.85 (2H, m)

### Reference Example 27

### 3-[[[[(3R)-3-[[(1,1-dimethylethoxy)carbonyl]amino]-1-piperidinyl]carbonyl]oxy]methyl]-2-methyl-1-[(2,3,4,9-tetrahydro-9-methyl-4-oxo-1H-carbazol-3-yl)methyl]-1H-imidazolium chloride

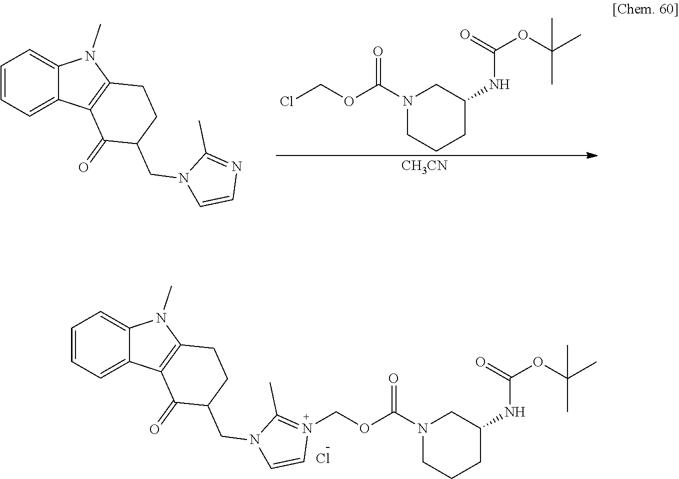

377 mg (1.28 mmol) of ondansetron was added at room temperature to an acetonitrile solution of 373 mg (1.27 mmol) of (3R)-3-[[(1,1-dimethylethoxy)carbonyl]amino]-1-piperidine carboxylic acid chloromethyl ester and stirred at 85° C. overnight. The reaction solution was condensed in a water bath set to 40° C. The residue was purified by silica gel column chromatography (10% methanol/chloroform) to obtain 697 mg (92%) of the title compound.

¹H-NMR (CDCl₃, δ): 1.32-1.57 (12H, m), 1.88-1.92 (1H, m), 2.03-2.11 (1H, m), 2.77-3.07 (5H, m), 3.12-3.35 (3H, m), 3.49-3.65 (2H, m), 3.70-3.95 (5H, m), 4.56-4.82 (3H, m), 6.06-6.21 (2H, m), 7.26-7.32 (3H, m), 7.39-7.45 (1H, m), 7.87 (1/2H, d, H=8 Hz), 7.96-7.80 (1/2H, m), 8.09-8.11 (1H, m)

### Reference Example 28

### 3-[[[[(3R)-3-amino-1-piperidinyl]carbonyl]oxy]methyl]-2-methyl-1-[(2,3,4,9-tetrahydro-9-methyl-4-oxo-1H-carbazol-3-yl)methyl]-1H-imidazolium chloride hydrochloride

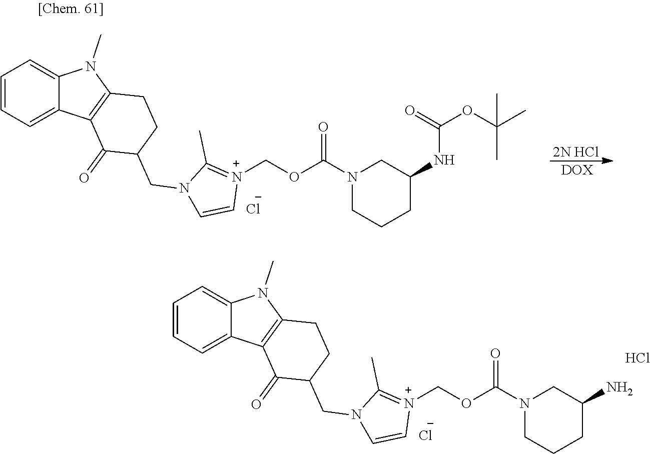

3 mL of 4 N hydrochloric acid/dioxane solution was added under cooling on ice to 3 mL of dioxane solution of 697 mg (L33 mmol) of 3-[[[[(3R)-3-[[(1,1-dimethylethoxy)carbonyl]amino]-1-piperidinyl]carbonyl]oxy]methyl]-2-methyl-1-[(2,3,4,9-tetrahydro-9-methyl-4-oxo-1H-carbazol-3-yl)methyl]-1H-imidazolium chloride. The reaction solution was warmed to room temperature and left to stand still for 1 hour. Thereafter, the solvent was distilled off under reduced pressure. Ethyl acetate was added to the residue and stirred and washed. Crystals were collected by filtration to obtain 611 mg (88%) of the title compound.

¹H-NMR (DMSO-d6, δ): 1.44-1.46 (1H, m), 1.56-1.61 (1H, 1.73-1.77 (1H, 1.91-1.97 (2H, m), 2.17-2.20 (1H, m), 2.78 (3/2H, s), 2.82 (3/2H, s), 3.02-3.19 (6H, m), 3.51-3.61 (1H, m), 3.75 (3H, s), 3.89-3.91 (1H, m), 4.34 (1H, dd, J=14, 7 Hz), 4.71-4.74 (1H, m), 6.08-6.09 (2H, m), 7.21-7.28 (2H, m), 7.56 (1H, d, J=8 Hz), 7.77 (1H, d, J=2 Hz), 7.80 (1H, d, J=2 Hz), 7.98 (1H, d, J=8 Hz), 8.36 (3/2H, br-s), 8.41 (3/2H, br-s)

### Example 9

### [(3R)-1-[[(ondansetron)methoxy]carbonyl]-3-piperidinyl]amino-chondroitin sulfate conjugate

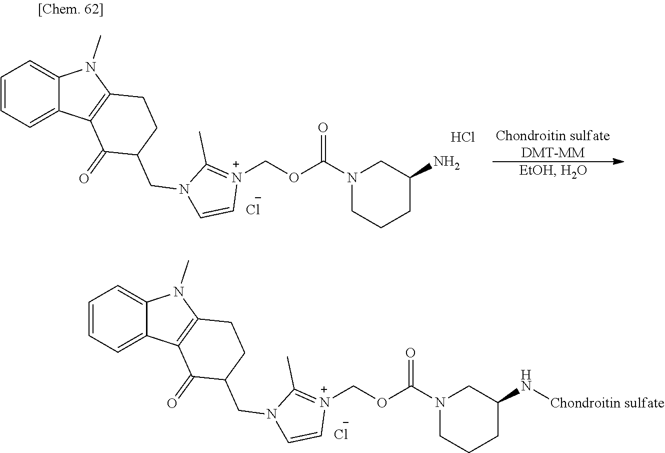

2 mL of ethanol was slowly added dropwise under stirring to 4.0 g (0.398 mmol) of 5% sodium chondroitin sulfate aqueous solution. To the mixed liquid, a solution of 42 mg (0.080 mmol) of 3-[[[[(3R)-3-amino-1-piperidinyl]carbonyl]oxy]methyl]-2-methyl-1-[(2,3,4,9-tetrabydro-9-methyl-4-oxo-1H-carbazol-3-yl)methyl]-1H-imidazolium chloride hydrochloride in 1 mL of ethanol was added, a solution of 38 mg (0.136 mmol) of 4-(4,6-dimethoxy-1,3,5-triazin-2-yl)-4-methylmorpholinium chloride (DMT-MM) in 1 mL of ethanol was then added, 1 mL of ethanol and 1 mL of water were further added, and the resultant liquid was stirred at room temperature overnight. 100 μL of 20% sodium chloride aqueous solution was added to the reaction solution, and ethanol was further added dropwise mL) until immediately before the reaction solution became cloudy. The reaction solution was added dropwise under stirring to 12 mL of 93% ethanol, and 5 mL of ethanol was added to the mixed liquid and stirred for 1 hour. Precipitates were isolated using a centrifuge, washed twice with 90% ethanol, twice with ethanol, and twice with diethyl ether. The obtained precipitates were dried overnight using a vacuum pump to obtain 209 mg of the title compound. Based on values of integral in ¹H-NMR, the introduction ratio of ondansetron per whole disaccharide unit (glucuronic acid) of chondroitin sulfate was 16 mol %.

### Reference Example 29

### N-[2-[[(1,1-dimethylethoxy)carbonyl]amino]ethyl]-N-methylcarbamic acid chloromethyl ester

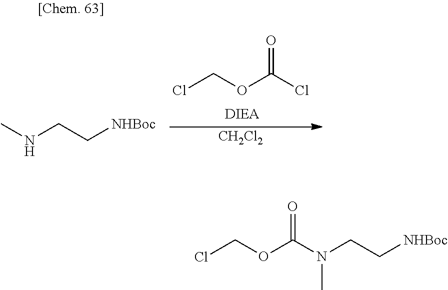

A methylene chloride solution of 0.612 mL (6.89 mmol) of chloromethyl chloroformate was added dropwise under cooling on ice to a methylene chloride solution of 1.00 g (5.74 mol) of N-([(1,1-dimethylethoxy)carbonyl])-N′-methylethylenediamine and 1.50 mL (8.61 mmol) of N,N-diisopropylethylamine. After 15 minutes, water was then added to the reaction solution, and the resultant liquid was extracted with diethyl ether. The obtained organic layer was washed with 5% potassium hydrogensulfate aqueous solution, saturated sodium bicarbonate water, and saturated saline and dried over anhydrous magnesium sulfate, and the solvent was distilled off under reduced pressure. The residue was purified by silica gel column chromatography to obtain 1.47 g (96%) of the title compound.

¹H-NMR (CDCl₃, δ): 1.44 (9H, s), 2.98 (3/2H, s), 3.00 (3/2H, s), 3.27-3.33 (2H, m), 3.39-3.47 (2H, m), 4.67 (1/2H, br-s), 4.79 (1/2H, br-s), 5.78 (1H, s), 5.79 (1H, s)

### Reference Example 30

### 3-[[[[[2-[[(1,1-dimethylethoxy)carbonyl]amino]ethyl]methylamino]carbonyl]oxy]methyl]-2-methyl-1-[(2,3,4,9-tetrahydro-9-methyl-4-oxo-1H-carbazol-3-yl)methyl]-1H-imidazolium chloride

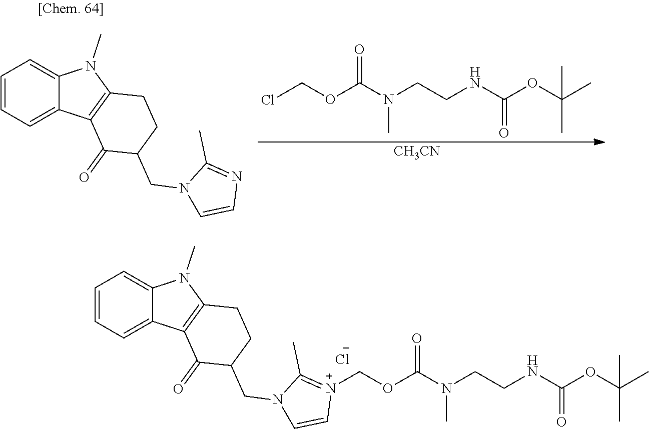

400 mg (1.36 mmol) of ondansetron was added at room temperature to an acetonitrile solution of 384 mg (1.36 mmol) of N-[2-[[(1,1-dimethylethoxy)carbonyl]amino]ethyl]-N-methylcarbamic acid chloromethyl ester and stirred at 85° C. overnight. The reaction solution was condensed in a water bath set to 40° C. The residue was purified by silica gel column chromatography (9→7% methanol/chloroform) to obtain 56 mg (7%) of the title compound.

¹H-NMR (CDCl₃, δ): 1.40 (9/2H, s), 1.41 (9/2H, s), 2.08 (1H, m), 2.76-2.80 (1H, m), 2.96 (3/2H, s), 2.98 (3/2H, s), 3.05-3.39 (10H, m), 3.71 (3H, s), 4.59-4.61 (1H, m), 4.75 (1/2H, dd, J=14, 7 Hz), 4.79 (1/2H, dd, J=14, 7 Hz), 5.02 (1/2H, br-s), 5.21 (1/2H, br-s), 6.08-6.16 (2H, m), 7.26-7.33 (3H, m), 7.42 (1/2H, d, J=2 Hz), 7.55 (1/2H, br-s), 7.84 (1H, br-s), 8.10 (1H, d, J=8 Hz)

### Reference Example 31

### 3-[[[[(2-aminoethyl)methylamino]carbonyl]oxy]methyl]-2-methyl-1-[(2,3,4,9-tetrahydro-9-methyl-4-oxo-1H-carbazol-3-yl)methyl]-1H-imidazolium chloride hydrochloride

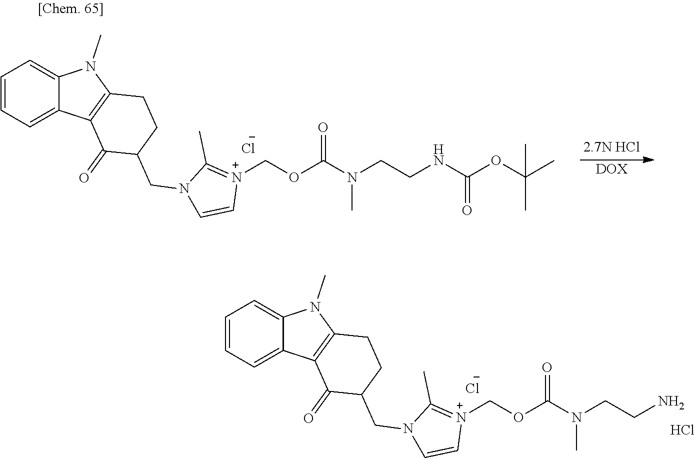

2 mL of 4 N hydrochloric acid/dioxane solution was added under cooling on ice to 1 mL of dioxane solution of 56 mg (0.10 mmol) of 3-[[[[[2-[[(1,1-dimethylethoxy)carbonyl]amino]ethyl]methylamino]carbonyl]oxy]methyl]-2-methyl-1-[(2,3,4,9-tetrahydro-9-methyl-4-oxo-1H-carbazol-3-yl)methyl]-1H-imidazolium chloride. The reaction solution was warmed to room temperature and left to stand still for 1 hour. Thereafter, the solvent was distilled off under reduced pressure. Ethyl acetate was added to the residue and stirred and washed. Crystals were collected by filtration to obtain 47 mg (95%) of the title compound.

¹H-NMR (DMSO-d6, δ): 1.94-2.02 (1H, m), 2.17-2.20 (1H, m), 2.79-3.05 (9H, m), 3.13-3.19 (2H, m), 3.45-3.52 (2H, m), 3.75 (3H, s), 4.34 (1H, dd, J=14, 7 Hz), 4.72 (1H, dd, J=14, 7 Hz), 6.02 (1H, s), 6.08 (1H, s), 7.21-7.29 (2H, m), 7.56 (1H, d, J=8 Hz), 7.76-7.77 (1H, m), 7.79 (1/2H, d, J=2 Hz), 7.82 (1/2H, d, J=2 Hz), 7.98 (1/2H, d, J=8 Hz), 7.99 (1/2H, d, J=8 Hz), 8.09 (3H, br-s)

### Example 10

### [2-[methyl[[(ondansetron)methoxy]carbonyl]amino]ethyl]amino-chondroitin sulfate conjugate

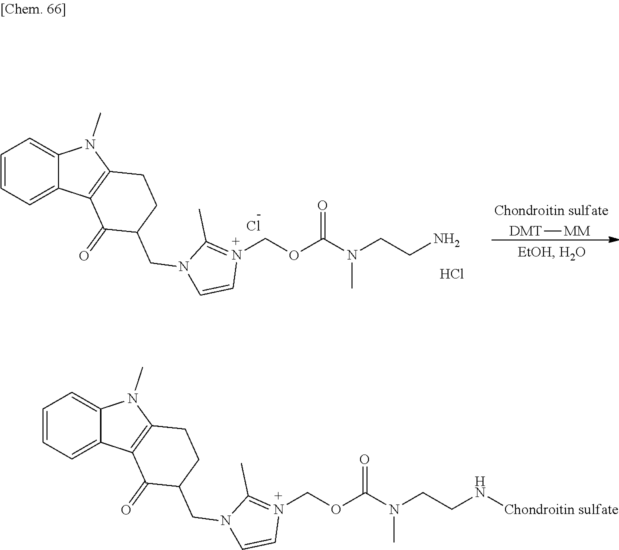

2 mL of ethanol was slowly added dropwise under stirring to 4.0 g (0.398 mmol) of 5% sodium chondroitin sulfate aqueous solution. To the mixed liquid, a solution of 40 mg (0.080 mmol) of 3-[[[[(2-aminoethyl)methylamino]carbonyl]oxy]methyl]-2-methyl-1-[(2,3,4,9-tetrahydro-9-methyl-4-oxo-1H-carbazol-3-yl)methyl]-1H-imidazolium chloride hydrochloride in 1 mL of ethanol was added, a solution of 38 mg (0.136 mmol) of 4-(4,6-dimethoxy-1,3,5-triazin-2-yl)-4-methylmorpholinium chloride (DMT-MM) in 1 mL of ethanol was then added, 1 mL of ethanol and 1 mL of water were further added, and the resultant liquid was stirred at room temperature overnight. 100 μL of 20% sodium chloride aqueous solution was added to the reaction solution, and ethanol was further added dropwise (2 mL) until immediately before the reaction solution became cloudy. The reaction solution was added dropwise under stirring to 12 mL of 93% ethanol, and 5 mL of ethanol was added to the mixed liquid and stirred for 1 hour. Precipitates were isolated using a centrifuge, washed twice with 90% ethanol, twice with ethanol, and twice with diethyl ether. The obtained precipitates were dried overnight using a vacuum pump to obtain 193 ME of the title compound. Based on values of integral in ¹H-NMR, the introduction ratio of ondansetron per whole disaccharide unit (glucuronic acid) of chondroitin sulfate was 18 mol %.

### Reference Example 32

### N-[3-[[(1,1-dimethylethoxy)carbonyl]amino]-2-methylpropyl]carbamic acid chloromethyl ester

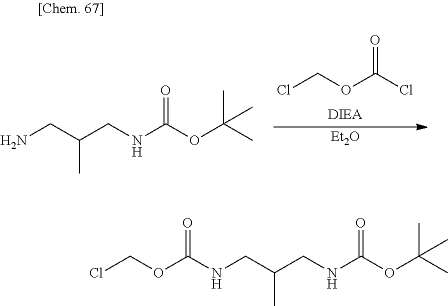

A diethyl ether solution of 829 mg (6.43 mmol) of chloromethyl chloroformate was added dropwise under cooling on ice to a diethyl ether solution of 1009 mg (5.36 mmol) of N-(3-amino-2-methylpropyl)carbamic acid 1,1-dimethyletyl ester and 1039 mg (8.04 mmol) of N,N-diisopropylethylamine. The temperature of the reaction solution was raised to room temperature and the reaction solution was stirred overnight. Water was added to the reaction solution, and then diethyl ether was distilled off under reduced pressure. After the resultant liquid was extracted with ethyl acetate, the obtained organic layer was washed with 10% potassium hydrogensulfate aqueous solution, saturated sodium bicarbonate water, and saturated saline and dried over anhydrous magnesium sulfate, and the solvent was distilled off under reduced pressure. The residue was purified by silica gel column chromatography (8→35% ethyl acetate/hexane) to obtain 1.19 g (79%) of the title compound.

¹H-NMR. (CDCl₃, δ): 0.89 (3H, d, J=7 Hz), 1.44 (9H, s), 1.77-1.86 (1H, m), 2.93-3.03 (2H, m), 3.17-3.27 (2H, m), 4.85 (1H, br-s), 5.74-5.79 (2H, m), 5.92 (1H, br-s)

### Reference Example 33

### 3-[[[[[3-[[(1,1-dimethylethoxy)carbonyl]amino]-2-methylpropyl]amino]carbonyl]oxy]methyl]-2-methyl-1-[(2,3,4,9-tetrabydro-9-methyl-4-tetrahydro-1H-carbazol-3-yl)methyl]-1H-imidazolium chloride

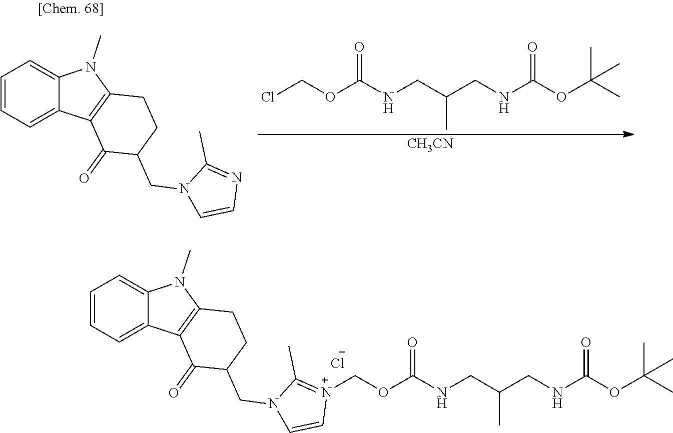 [SMILES: Cc1nccn1CC1CCc2c(c3ccccc3n2C)C1=O ; CC(CNC(=O)OCCl)CNC(=O)OC(C)(C)C ; "CH3CN" ; Cc1n(CC2CCc3c(c4ccccc4n3C)C2=O)cc[n+]1COC(=O)NCC(C)CNC(=O)OC(C)(C)C ; [Cl-]]

386 mg (1.31 mmol) of ondansetron was added at room temperature to an acetonitrile solution of 369 mg (1.31 mmol) of N-[3-[[(1,1-dimethylethoxy)carbonyl]amino]-2-methylpropyl]carbamic acid chloromethyl ester and stirred at 85° C. overnight. The reaction solution was filtered, and then the filtrate was condensed in a water bath set to 40° C. The residue was purified by silica gel column chromatography (10→420% methanol/chloroform) to obtain 309 mg (41%) of the title compound.

¹H-NMR. (CDCl₃, δ): 0.84 (3/2H, d, J=7 Hz), 0.85 (3/2H, d, J=7 Hz), 1.42 (9H, s), 1.82-1.83 (1H, m), 2.03 (1H, qd, J=12, 6 Hz), 2.70-2.72 (1H, m), 2.91-2.99 (2H, m), 3.05 (3H, s), 3.09-3.29 (5H, m), 3.68 (3H, s), 4.59 (1/2H, d, J=14 Hz), 4.60 (1/2H, d, J=14 Hz), 4.74 (1H, dd, J=14, 7 Hz), 5.16 (1H, br-s), 6.02-6.12 (2H, m), 7.14 (1H, t, J=6 Hz), 7.24-7.32 (3H, m), 7.48 (1H, d, J=2 Hz), 7.90 (1H, br-s), 8.09-8.11 (1H, m)

### Reference Example 34

### 3-[[[[(3-amino-2-methylpropyl)amino]carbonyl]oxy]methyl]-2-methyl-1-[(2,3,4,9-tetrahydro-9-methyl-4-oxo-1H-carbazol-3-yl)methyl]-1H-imidazolium chloride hydrochloride

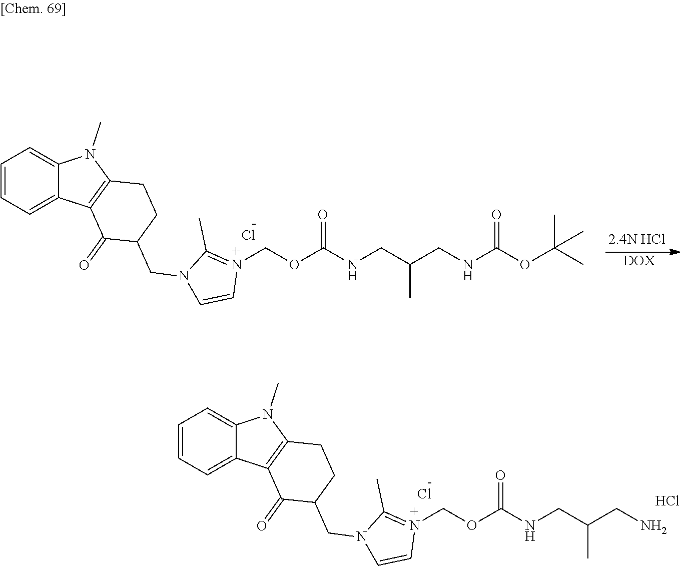

3 mL of 4 N hydrochloric acid/dioxane solution was added under cooling on ice to 2 mL of dioxane solution of 309 mg (0.66 mmol) of 3-[[[[[3-[[(1,1-dimethylethoxy)carbonyl]amino]-2-methylpropyl]amino]carbonyl]oxy]methyl]-2-methyl-1-[(2,3,4,9-tetrahydro-9-methyl-4-tetrahydro-1H-carbazol-3-yl)methyl]-1H-imidazolium chloride. The reaction solution was warmed to room temperature and left to stand still for 1 hour. Thereafter, the solvent was distilled off under reduced pressure. Ethyl acetate was added to the residue and stirred and washed. Crystals were collected by filtration to obtain 297 mg (quantitative) of the title compound.

¹H-NMR. (DMSO-d6, δ): 0.89 (3H, d, J=7 Hz), 1.91-2.00 (2H, m), 2.14-2.17 (1H, m), 2.54-2.57 (1H, m), 2.76-2.78 (4H, m), 2.92-3.04 (3H, m), 3.13-3.19 (2H, m), 3.75 (3H, s), 4.35 (1H, dd, J=14, 7 Hz), 4.72 (1H, dd, J=14, 7 Hz), 6.06 (2H, s), 7.22 (1H, t, J=8 Hz), 7.27 (1H, t, J=8 Hz), 7.56 (1H, d, J=8 Hz), 7.77 (1H, s), 7.79 (1H, s), 7.91 (1H, t, J=6 Hz), 7.98 (1H, d, J=8 Hz), 8.15 (3H, br-s)

### Example 11

### [2-methyl-3-[[[(ondansetron)methoxy]carbonyl]amino]propyl]amino-chondroitin sulfate conjugate

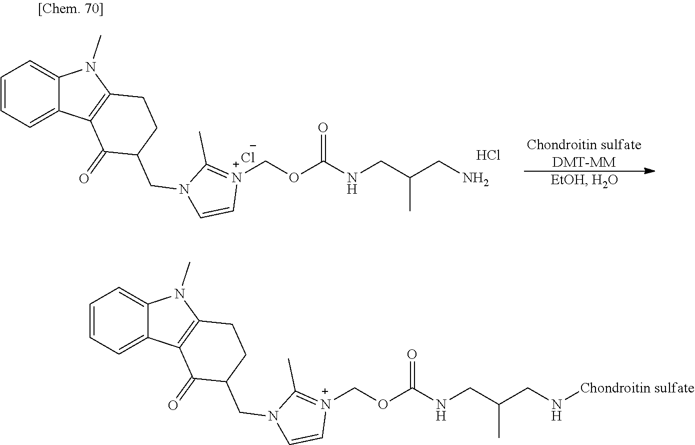

2 mL of ethanol was slowly added dropwise under stirring to 4.0 g (0.398 mmol) of 5% sodium chondroitin sulfate aqueous solution. To the mixed liquid, a solution of 41 mg (0.080 mmol) of 3-[[[[(3-amino-2-methylpropyl)amino]carbonyl]oxy]methyl]-2-methyl-1-[(2,3,4,9-tetrahydro-9-methyl-4-oxo-1H-carbazol-3-yl)methyl]-1H-imidazolium chloride hydrochloride in 1 mL of ethanol was added, a solution of 38 mg (0.136 mmol) of 4-(4,6-dimethoxy-1,3,5-triazin-2-yl)-4-methylmorpholinium chloride (DMT-MM) in 1 mL of ethanol was then added, 1 mL of ethanol and 1 mL of water were further added, and the resultant liquid was stirred at room temperature overnight. 100 μL of 20% sodium chloride aqueous solution was added to the reaction solution, and ethanol was further added dropwise (2 mL) until immediately before the reaction solution became cloudy. The reaction solution was added dropwise under stirring to 12 mL of 93% ethanol, and 5 mL of ethanol was added to the mixed liquid and stirred for 1 hour. Precipitates were isolated using a centrifuge, washed twice with 90% ethanol, twice with ethanol, and twice with diethyl ether. The obtained precipitates were dried overnight using a vacuum pump to obtain 199 mg of the title compound. Based on values of integral in ¹H-NMR, the introduction ratio of ondansetron per whole disaccharide unit (glucuronic acid) of chondroitin sulfate was 17 mol %.

### Reference Example 35

### N-[6-[[(1,1-dimethylethoxy)carbonyl]amino]hexyl]carbamic acid 1-chloroethyl ester

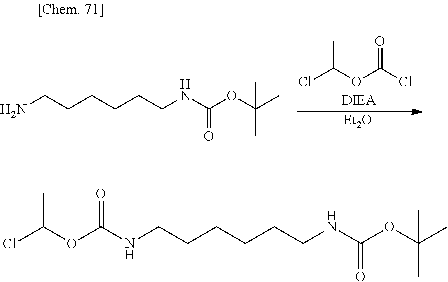

A methylene chloride solution of 793 mg (5.55 mmol) of 1-chloroethyl chloroformate was added dropwise under cooling on ice to a methylene chloride solution of 1.00 g (6.24 mmol) of N-[(1,1-dimethylethoxy)carbonyl]-1,6-hexariediamine and 1.00 g (4.62 mmol) of N,N-diisopropylethylamine. The reaction solution was stirred at room temperature overnight, water was then added to the reaction solution, and the resultant liquid was extracted with ethyl acetate. The obtained organic layer was washed with 10% potassium hydrogensulfate aqueous solution, saturated sodium bicarbonate water, and saturated saline and dried over anhydrous magnesium sulfate, and the solvent was distilled off under reduced pressure. The residue was purified by silica chromatography (5-4.20% ethyl acetate/hexane) to obtain 97 mg (7%) of the title compound.

¹H-NMR (CDCl₃, δ): 1.33-1.35 (5H, m), 1.44-1.54 (12H, m), 1.75 (3H, d, J=6 Hz), 3.10-3.12 (2H, m), 3.19-3.23 (2H, m), 4.52 (1H, br-s), 4.93 (1H, br-s), 6.57 (1H, q, J=6 Hz)

### Reference Example 36

### 3-[1-[[[[6-[[(1,1-dimethylethoxy)carbonyl]amino]hexyl]amino]carbonyl]oxy]ethyl]-2-methyl-1-[(2,3,4,9-tetrahydro-9-methyl-4-oxo-1H-carbazol-3-yl)methyl]-1H-imidazolium chloride

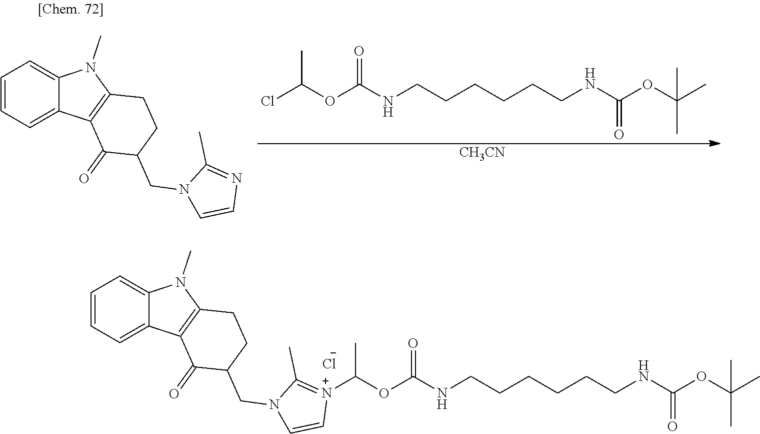 [SMILES: Cc1nccn1CC1CCc2c(c3ccccc3n2C)C1=O ; CC(Cl)OC(=O)NCCCCCCNC(=O)OC(C)(C)C ; "CH3CN" ; Cc1n(CC2CCc3c(c4ccccc4n3C)C2=O)cc[n+]1C(C)OC(=O)NCCCCCCNC(=O)OC(C)(C)C ; [Cl-]]

31.9 mg (1.09 mmol) of ondansetron was added at room temperature to an acetonitrile solution of 351 mg (L09 mmol) of N-[6-[[(1,1-dimethylethoxy)carbonyl]amino]hexyl]carbamic acid 1-chloroethyl ester and stirred at 85° C. overnight. The reaction solution was filtered, and then the filtrate was condensed in a water bath set to 40° C. The residue was purified by silica gel column chromatography (5→10% methanol/chloroform) to obtain 93 mg (14%) of the title compound.

¹H-NMR (CDCl₃, δ): 1.30-1.31 (5H, m), 1.44-1.51 (12H, m), 1.89 (3H, d, J=6 Hz), 2.07 (1H, qd, J=14, 5 Hz), 2.81-2.83 (1H, m), 3.04-3.17 (8H, m), 3.26-3.49 (2H, m), 3.70 (3H, s), 4.58-4.70 (3H, m), 5.78 (1H, t, J=6 Hz), 6.66 (1H, q, J=6 Hz), 7.24-7.33 (3H, m), 7.45 (1H, br-s), 8.08-8.10 (1H, m), 8.23-8.24 (1H, m)

### Reference Example 37

### 3-[1-[[[(6-aminohexyl)amino]carbonyl]oxy]ethyl]-2-methyl-1-[(2,3,4,9-tetrahydro-9-methyl-4-oxo-1H-carbazol-3-yl)methyl]-1H-imidazolium chloride hydrochloride

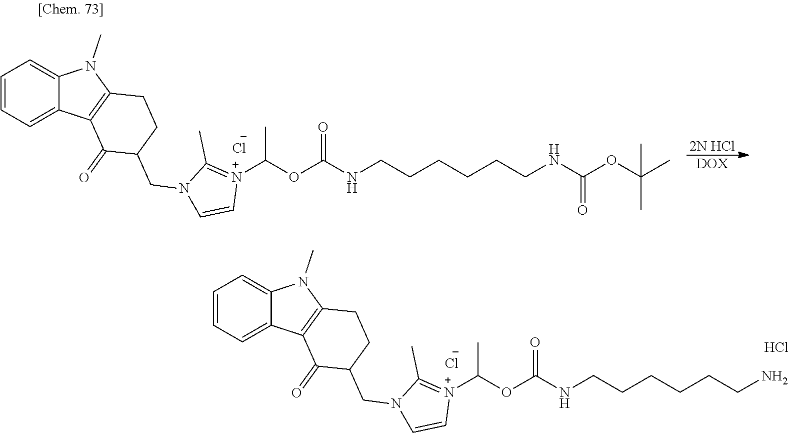

1 mL of 4 N hydrochloric acid/dioxane solution was added under cooling on ice to 1 mL of dioxane solution of 93 mg (0.15 mmol) of 3-[1-[[[[6-[[(1,1-dimethylethoxy)carbonyl]amino]hexyl]amino]carbonyl]oxy]ethyl]-2-methyl-1-[(2,3,4,9-tetrahydro-9-methyl-4-oxo-1H-carbazol-3-yl)methyl]-1H-imidazolium chloride. The reaction solution was warmed to room temperature and left to stand still for 1 hour. Thereafter, the solvent was distilled off under reduced pressure. Ethyl acetate was added to the residue and stirred and washed. Crystals were collected by filtration to obtain 84 mg (quantitative) of the title compound.

¹H-NMR (DMSO-d6, δ): 1.16-1.30 (4H, m), 1.34-1.40 (2H, m), 1.50-1.56 (2H, m), 1.73 (3H, d, J=6 Hz), 1.94-2.12 (2H, m), 2.70-2.74 (2H, m), 2.81 (3H, s), 2.89-3.19 (5H, m), 3.75 (3H, s), 4.36 (1H, dd, J=14, 8 Hz), 4.71-4.75 (1H, m), 6.74 (1H, q, J=6 Hz), 7.22 (1H, t, J=8 Hz), 7.27 (1H, t, J=8 Hz), 7.56 (1H, d, J=8 Hz), 7.72 (1H, t, J=6 Hz), 7.88 (1H, d, J=2 Hz), 7.93 (1H, d, J=2 Hz), 7.99 (1H, d, J=8 Hz), 8.04 (3H, br-s)

### Example 12

### [6-[[[1-(ondansetron)ethoxy]carbonyl]amino]hexyl]amino-chondroitin sulfate conjugate

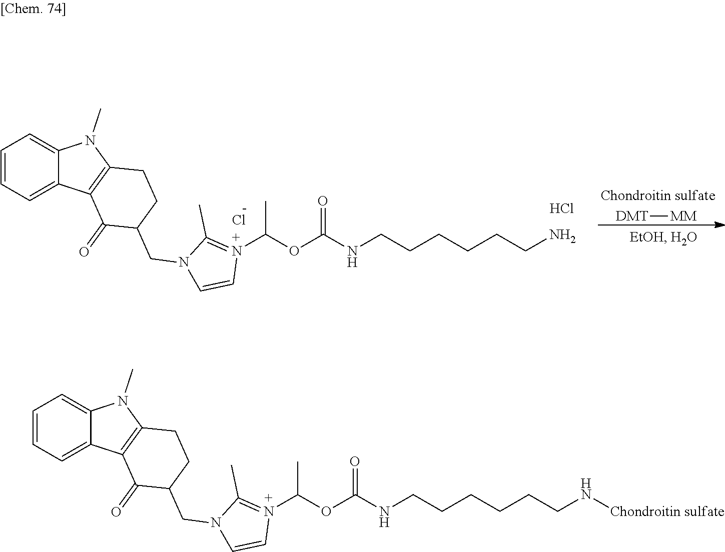

2 mL of ethanol was slowly added dropwise under stirring to 4.0 g (0.398 mmol) of 5% sodium chondroitin sulfate aqueous solution. To the mixed liquid, a solution of 44 mg (0.080 mmol) of 3-[1-[[[(6-aminohexyl)amino]carbonyl]oxy]ethyl]-2-methyl-1-[(2,3,4,9-tetrahydro-9-methyl-4-oxo-1H-carbazol-3-yl)methyl]-1H-imidazolium chloride hydrochloride in 1 mL of ethanol was added, a solution of 38 mg (0.136 mmol) of 4-(4,6-dimethoxy-1,3,5-triazin-2-yl)-4-methylmorpholinium chloride (DMT-MM) in 1 mL of ethanol was then added, 1 mL of ethanol and 1 mL of water were further added, and the resultant liquid was stirred at room temperature overnight. 100 μL, of 20% sodium chloride aqueous solution was added to the reaction solution, and ethanol was further added dropwise (2.3 mL) until immediately before the reaction solution became cloudy. The reaction solution was added dropwise under stirring to 12 mL of 93% ethanol, and 4.5 mL of ethanol was added to the mixed liquid and stirred for 1 hour. Precipitates were isolated using a centrifuge, washed twice with 90% ethanol, twice with ethanol, and twice with diethyl ether. The obtained precipitates were dried overnight using a vacuum pump to obtain 196 mg of the title compound. Based on values of integral in ¹H-NMR, the introduction ratio of ondansetron per whole disaccharide unit (glucuronic acid) of Chondroitin sulfate was 12 mol %.

### Reference Example 38

### Carbonic acid chloromethyl 2-[[(1,1-dimethylethoxy)carbonyl]amino]ethyl ester

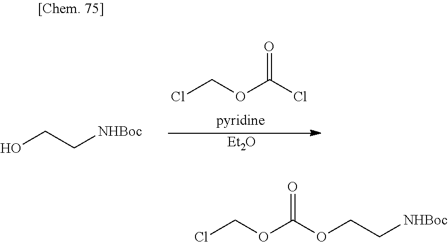 [SMILES: "Et2O" ; O=C(Cl)OCCl ; CC(C)(C)OC(=O)NCCO ; CC(C)(C)OC(=O)NCCOC(=O)OCCl]

1.32 mL (14.9 mmol) of methyl chloroformate was added at −15° C. to a diethyl ether (30 mL) solution of 2.00 g (12.4 mmol) of N-(2-hydroxyethyl)carbamic acid 1,1-dimethylethyl ester, and subsequently, a diethyl ether (15 mL) solution of 1.20 mL (14.9 mmol) of pyridine was added dropwise. The reaction solution was warmed to room temperature and stirred for 6 hours, water was then added to the reaction solution, and the resultant liquid was extracted with diethyl ether. The obtained organic layer was washed with saturated ammonium chloride aqueous solution, saturated sodium bicarbonate water, and saturated saline and dried over anhydrous magnesium sulfate, and the solvent was distilled off under reduced pressure. The residue was purified by silica gel column chromatography to obtain 2.63 g (84%) of the title compound.

¹H-NMR (CDCl₃, δ): 1.45 (9H, s), 3.44-3.45 (2H, m), 4.29 (2H, t, J=5 Hz), 4.82 (1H, br-s), 5.74 (2H, s)

### Reference Example 39

### 3-[[[[2-[[(1,1-dimethylethoxy)carbonyl]amino]ethoxy]carbonyl]oxy]methyl]-methyl-1-[(2,3,4,9-tetrahydro-9-methyl-4-oxo-1H-carbazol-3-yl)methyl]-1H-imidazolium iodide

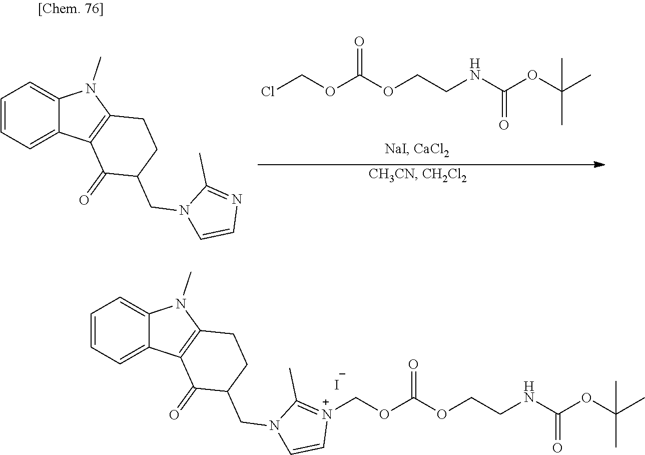

924 mg (3.15 mmol) of ondansetron, 944 mg (6.30 mmol) of sodium iodide, and 105 mg (0.95 mmol) of calcium chloride were added at room temperature to a mixed solution of dichloromethane and acetonitrile of 800 mg (3.15 mmol) of carbonic acid chloromethyl 2-[[(1,1-dimethylethoxy)carbonyl]amino]ethyl ester and stirred at 45° C. for 6 hours. The reaction solution was filtered, and then the reaction solution was condensed in a water bath set to 40° C. The residue was purified by silica gel column chromatography (10→15% methanol/chloroform) to obtain 480 mg (24%) of the title compound,

¹H-NMR (CDCl₃, δ): 1.42 (9H, s), 2.04 (1H, qd, J=12, 5 Hz), 2.74-2.79 (1H, m), 3.05-3.11 (4H, m), 3.27-3.43 (4H, m), 3.69 (3H, s), 4.26 (2H, t, J=5 Hz), 4.36 (1H, dd, J=14, 6 Hz), 4.86 (1H, dd, J=14, 6 Hz), 4.95 (1H, br-s), 6.11 (1H, d, J=12 Hz), 6.15 (1H, d, J=12 Hz) 7.26-7.32 (3H, m), 7.46 (1H, d, J=2 Hz), 7.68 (1H, d, J=2 Hz), 8.10-8.12 (1H, m)

### Reference Example 40

### 3-[[[(2-aminoethoxy)carbonyl]oxy]methyl]-2-methyl-1-[(2,3,4,9-tetrahydro-9-methyl-4-oxo-1H-carbazol-3-yl)methyl]-1H-imidazolium chloride hydrochloride

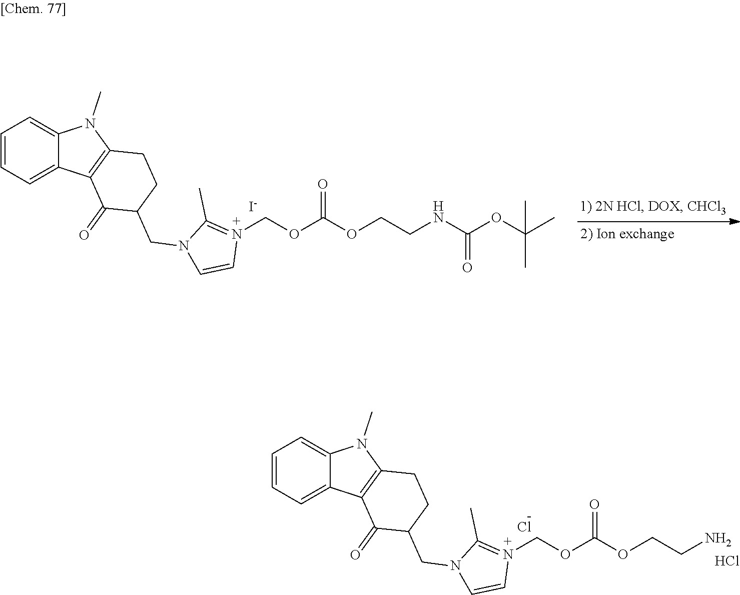

2 mL of 4N hydrochloric acid/dioxane solution was added under cooling on ice to 2 mL of chloroform solution of 424 mg (0.66 mmol) of 3-[[[[2-[[(1,1-dimethylethoxy)carbonyl]amino]ethoxy]carbonyl]oxy]methyl]-2-methyl-1-[(2,3,4,9-tetrahydro-9-methyl-4-oxo-1H-carbazol-3-yl)methyl]-1H-imidazolium iodide. The reaction solution was warmed to room temperature and left to stand still for 30 minutes. Thereafter, the solvent was distilled off under reduced pressure. The residue was dissolved in methanol and allowed to pass through Cl type ion exchange resin (DOWEX (registered trademark) 1×4 100-200 mesh), and the eluate was condensed under reduced pressure. Ethyl acetate was added to the residue and stirred for 1.5 hours. The precipitated crystals were collected by filtration to obtain 295 mg (92%) of the title compound.

¹H-NMR (DMSO-d6, δ): 1.99 (1H, qd, J=12, 5 Hz), 2.19-2.22 (1H, m), 2.80 (3H, s), 2.98-3.05 (1H, m), 3.12-3.21 (4H, m), 3.75 (3H, s), 4.34 (1H, dd, J=14, 7 Hz), 4.40 (2H, t, J=6 Hz), 4.73 (1H, dd, J=14, 7 Hz), 6.21 (1H, d, J=12 Hz), 6.23 (1H, d, J=12 Hz), 7.20-7.28 (2H, m), 7.56 (1H, d, J=8 Hz), 7.82 (1H, d, J=2 Hz), 7.88 (1H, d, J=2 Hz), 7.99 (1H, d, J=8 Hz), 8.50 (3H, br-s)

### Example 13

### [2-[[[(ondansetron)methoxy]carbonyl]oxy]ethyl]amino-chondroitin sulfate conjugate

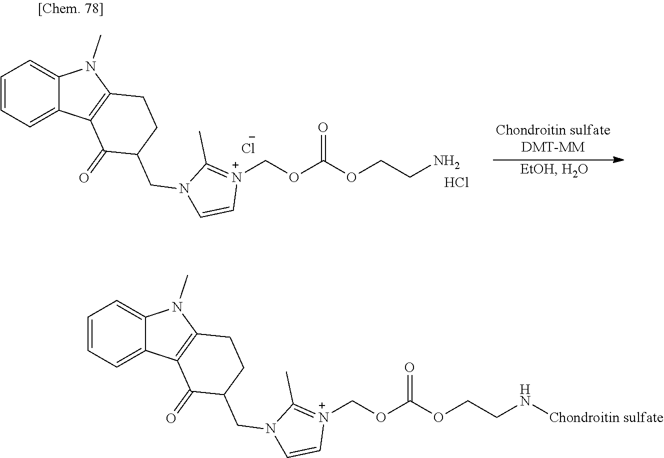

2 mL of ethanol was slowly added dropwise under stirring to 4.0 g (0.398 mmol) of 5% sodium chondroitin sulfate aqueous solution. To the mixed liquid, 1 mL of an aqueous solution of 46 mg (0,094 mmol) of 3-[[[(2-aminoethoxy)carbonyl]oxy]methyl]-2-methyl-1-[(2,3,4,9-tetrahydro-9-methyl-4-oxo-1H-carbazol-3-yl)methyl]-1H-imidazolium chloride hydrochloride was added, 1 mL of an aqueous solution of 38 mg (0.136 mmol) of 4-(4,6-dimethoxy-1,3,5-triazin-2-yl)-4-methylmorpholinium chloride (DMT-MM) was then added, 4 mL of ethanol was further added, and the resultant liquid was stirred at room temperature overnight. 100 μL of 20% sodium chloride aqueous solution was added to the reaction solution, and ethanol was further added dropwise (3 until immediately before the reaction solution became cloudy. The reaction solution was added dropwise under stirring to 14.5 mL of 93% ethanol, and 5.5 mL of ethanol was added to the mixed liquid and stirred for 1 hour. Precipitates were isolated using a centrifuge, washed twice with 90% ethanol, twice with ethanol, and twice with diethyl ether. The obtained precipitates were dried overnight using a vacuum pump to obtain 257 mg of the title compound. Based on values of integral in ¹H-NMR, the introduction ratio of ondansetron per whole disaccharide unit (glucuronic acid) of chondroitin sulfate was 17 mol %.

### Reference Example 41

### 1-[2-[(4-chlorophenyl)phenyl methoxy]ethyl]-1-[[[[3-[[(1,1-dimethylethoxy)carbonyl]amino]propoxy]carbonyl]oxy]methyl]piperidinium chloride

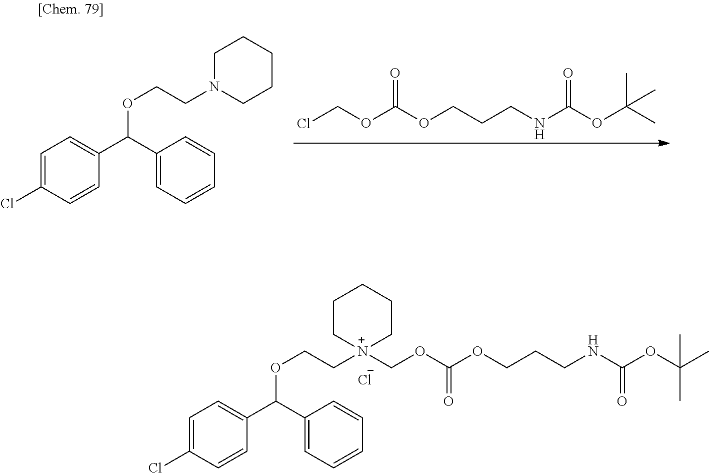 [SMILES: Clc1ccc(C(OCCN2CCCCC2)c2ccccc2)cc1 ; CC(C)(C)OC(=O)NCCCOC(=O)OCCl ; CC(C)(C)OC(=O)NCCCOC(=O)OC[N+]1(CCOC(c2ccccc2)c2ccc(Cl)cc2)CCCCC1 ; [Cl-]]

680 mg (2.06 mmol) of cloperastine was added at room temperature to a dichloromethane solution of 368 mg (1.37 mmol) of carbonic acid chloromethyl. 3-[[(1,1-dimethylethoxy)carbonyl]amino]propyl ester, and the reaction solution was condensed at 70′C over 1 hour and then stirred at the same temperature overnight. The residue was purified by silica gel column chromatography (2>10% methanol/chloroform) to obtain 335 mg (41%) of the title compound.

¹H-NMR (CDCl₃, δ): 1.42 (9H, s), 1.76-1.97 (8H, m), 3.22 (2H, q, J=7 Hz), 3.71-3.78 (2H, m), 3.93-4.05 (4H, m), 4.22-4.26 (4H, m), 4.71 (1H, br-s), 5.46 (1H, s), 5.82 (1H, d, J=12 Hz), 5.84 (1H, d, J=12 Hz), 7.24-7.36 (9H, m)

### Reference Example 42

### 1-[[[(3-aminopropoxy)carbonyl]oxy]methyl]-1-[2-[(4-chlorophenyl)phenylmethoxy]ethyl]piperidinium chloride hydrochloride

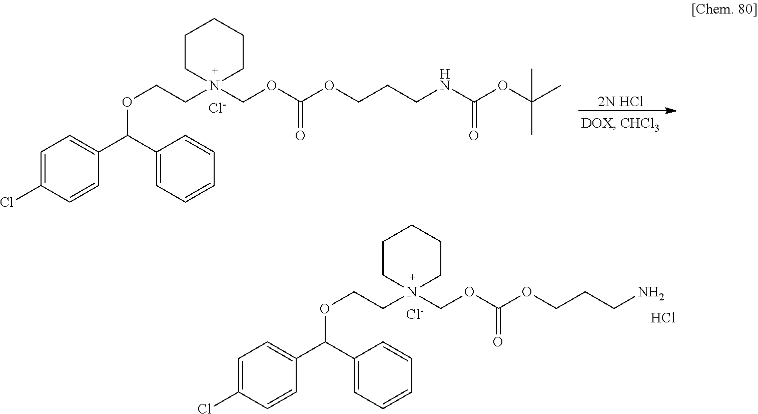

1 mL of 4 N hydrochloric acid/dioxane solution was added under cooling on ice to 1 mL of chloroform solution of 335 mg (0.56 mmol) of 1-[2-[(4-chlorophenyl)phenyl methoxy]ethyl]-1-[[[[3-[[(1,1-dimethylethoxy)carbonyl]amino]propoxy]carbonyl]oxy]methyl]piperidinium chloride. The reaction solution was warmed to room temperature and left to stand still for 1 hour. Thereafter, the solvent was distilled off under reduced pressure. Ethyl acetate was added to the residue and stirred for 1 hour. Crystals were collected by filtration to obtain 248 tug (83%) of the title compound.

¹H-NMR (DMSO-d6, δ): 1.51-1.63 (2H, m), 1.78-1.99 (6H, m), 2.86 (2H, q, J=6 Hz), 3.47-3.52 (4H, m), 3.79-3.81 (4H, m), 4.23 (2H, t, J=6 Hz), 5.48 (2H, s), 5.64 (1H, s), 7.28-7.43 (9H, m), 8.21-8.27 (3H, m)

### Example 14

### [3-[[[(cloperastine)methoxy]carbonyl]oxy]propyl]amino-chondroitin sulfate conjugate

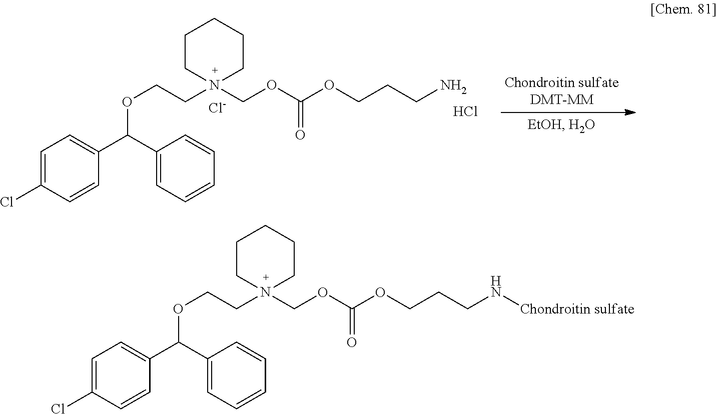

2 mL of ethanol was slowly added dropwise under stirring to 4.0 g (0.398 mmol) of 5% sodium chondroitin sulfate aqueous solution. To the mixed liquid, a solution of 43 mg (0.080 mmol) of 1-[[[(3-aminopropoxy)carbonyl]oxy]methyl]-1-[2-[(4-chlorophenyl)phenyl methoxy]ethyl]piperidinium chloride hydrochloride in 1 mL of ethanol was added, a solution of 38 mg (0.136 mmol) of 4-(4,6-dimethoxy-1,3,5-triazin-2-yl)-4-methylmorpholinium chloride (DMT-MM) in 1 mL of ethanol was then added, 1 mL of ethanol and 1 mL of water were further added, and the resultant liquid was stirred at room temperature overnight. 100 μL of 20% sodium chloride aqueous solution was added to the reaction solution, and ethanol was further added dropwise (3 mL) until immediately before the reaction solution became cloudy. The reaction solution was added dropwise under stirring to 12 mL of 93% ethanol, and 4 mL of ethanol was added to the mixed liquid and stirred for 1 hour. Precipitates were isolated using a centrifuge, washed twice with 90% ethanol, twice with ethanol, and twice with diethyl ether. The obtained precipitates were dried overnight using a vacuum pump to obtain 196 mg of the title compound. Based on values of integral in ¹H-NMR, the introduction ratio of cloperastine per whole disaccharide unit (glucuronic acid) of chondroitin sulfate was 7 mol %.

### Reference Example 43

### N-[[[[3-[[(1,1-dimethylethoxy)carbonyl]amino]propoxy]carbonyl]oxy]ethyl]-N,N,α-trimethyl-10H-phenothiazin-10-ethanaininium chloride

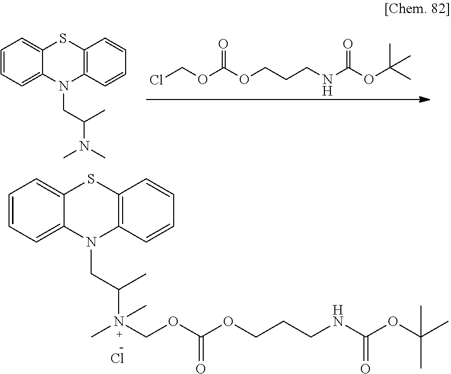

623 mg (2.19 mmol) of promethazine was added to a dichloromethane solution of 391 mg (1.46 mmol) of carbonic acid chloromethyl 3-[[(1,1-dimethylethoxy)carbonyl]amino]propyl ester, and the reaction solution was condensed at 70° C. over 1 hour and then stirred at the same temperature overnight. The residue was purified by silica gel column chromatography (5->15% methanol/chloroform) to obtain 247 rug (31%) of the title compound.

¹H-NMR (CDCl₃, δ): 1.20-1.23 (3H, m), 1.42 (9H, s), 1.86 (2H, quire, 7 Hz), 3.13-3.20 (2H, m), 3.52 (3H, s), 3.64 (3H, s), 3.69-3.71 (1H, m), 3.89-4.01 (1H, m), 4.19-4.29 (2H, m), 4.90 (1H, dd, J=15.5 Hz), 5.01 (1H, br-s), 5.97 (1H, d, J=9 Hz), 6.13 (1H, d, J=9 Hz), 6.97-7.02 (3H, m), 7.19-7.26 (5H, m)

### Reference Example 44

### N-[[[(3-aminopropoxy)carbonyl]oxy]methyl]-N,N,α-trimethyl-10H-phenothiazin-10-ethanaminium chloride hydrochloride

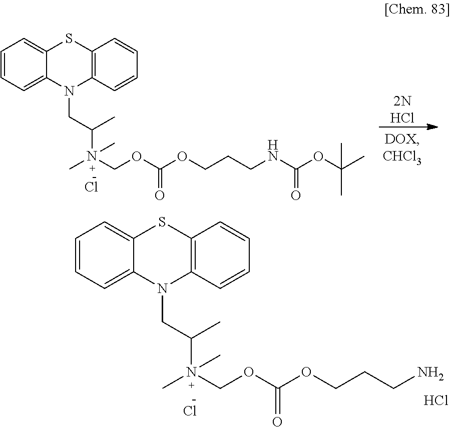

2 mL of 4 N hydrochloric acid/dioxane solution was added under cooling on ice to 2 mL of chloroform solution of 247 mg (0.45 mmol) of N-[[[[3-[[(1,1-dimethylethoxy)carbonyl]amino]propoxy]carbonyl]oxy]methyl]-N,N,α-trimethyl-10H-phenothiazin-10-ethanaminium chloride. The reaction solution was warmed to room temperature and left to stand still for 1 hour. Thereafter, the solvent was distilled off under reduced pressure. Ethyl acetate was added to the residue and stirred for 1 hour. Crystals were collected by filtration to obtain 158 mg (72%) of the title compound.

¹H-NMR (DMSO-d6, δ): 1.43 (3H, d, J=7 Hz), 1.98-2.02 (2H, m), 2.83-2.88 (2H, m), 3.15-3.22 (6H, m), 3.90-3.91 (1H, m), 4.13-4.17 (1H, m), 4.24-4.32 (2H, m), 4.66-4.69 (1H, m), 5.48 (1H, d, J=9 Hz), 5.51 (1H, d, J=9 Hz), 7.02-7.09 (3H, m), 7.24-7.34 (5H, m), 8.26 (3H, br-s)

### Example 15

### [3-[[[(promethazine)methoxy]carbonyl]oxy]propyl]amino-chondroitin sulfate conjugate

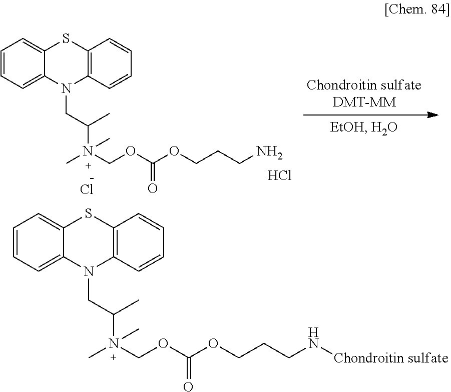

2 mL of ethanol was slowly added dropwise under stirring to 4.0 g (0.398 mmol) of 5% sodium chondroitin sulfate aqueous solution, To the mixed liquid, a solution of 39 mg (0.080 mmol) of N-[[[(3-aminopropoxy)carbonyl]oxy]methyl]-N,N,α-trimethyl-10H-phenothiazin-10-ethanaminium chloride hydrochloride in 1 mL of ethanol was added, a solution of 38 mg (0.136 mmol) of 4-(4,6-dimethoxy-1,3,5-triazin-2-yl)-4-methylmorpholinium chloride (DMT-MM) in 1 mL of ethanol was then added, 1 mL of ethanol and 1 mL of water were further added, and the resultant liquid was stirred at room temperature overnight. 100 μL of 20% sodium chloride aqueous solution was added to the reaction solution, and ethanol was further added dropwise (2.5 mL) until immediately before the reaction solution became cloudy. The reaction solution was added dropwise under stirring to 12 mL of 93% ethanol, and 4.5 mL of ethanol was added to the mixed liquid and stirred for 1 hour. Precipitates were isolated using a centrifuge, washed twice with 90% ethanol, twice with ethanol, and twice with diethyl ether. The obtained precipitates were dried overnight using a vacuum pump to obtain 196 mg of the title compound. Based on values of integral in H-NMR, the introduction ratio of promethazine per whole disaccharide unit (glucuronic acid) of chondroitin sulfate was 6 mol %.

### Reference Example 45

### Carbonic acid 1-chloroethyl 2[[(1,1-dimethylethoxy)carbonyl]amino]-1-methylethyl ester

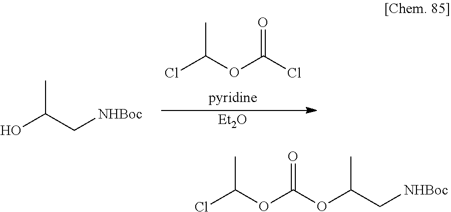

1.33 mL (12.2 mmol) of 1-chloroethyl chloroformate was added at −15° C. to a diethyl ether (1.5 mL) solution of 1.78 g (10.2 mmol) of N-(2-hydroxypropyl)carbamic acid 1,1-dimethylethyl ester, and subsequently, a diethyl ether (10 mL) of 1.23 mL (15.2 mmol) of pyridine was added dropwise. The reaction solution was warmed to room temperature and stirred for 6 hours, water was then added to the reaction solution, and the resultant liquid was extracted with diethyl ether. The obtained organic layer was washed with saturated ammonium chloride aqueous solution, saturated sodium bicarbonate water, and saturated saline and dried over anhydrous magnesium sulfate, and the solvent was distilled off under reduced pressure. The residue was purified by silica gel column chromatography to obtain 2.2.5 g (79%) of the title compound.

¹H-NMR (CDCl₃, δ): 1.31 (1.5H, d, J=6 Hz), 1.32 (1.5H, d, J=6 Hz), 1.44 (9H, s), 1.83 (1.5H, d, J=6 Hz), 1.84 (1.5H, d, J=6 Hz), 3.22-3.36 (1H, m), 3.41-3.43 (1H, m), 4.80 (1H, br-s), 4.87-4.91 (1H, m), 6.43 (1H, q, J=6 Hz)

### Reference Example 46

### 3-[1-[[[2-[[(1,1-dimethylethoxy)carbonyl]amino]-1-methylethoxy]carbonyl]oxy]ethyl]-2-methyl-1-[(2,3,4,9-tetrahydro-9-methyl-4-oxo-1H-carbazol-3-yl)methyl]-1H-imidazolium iodide

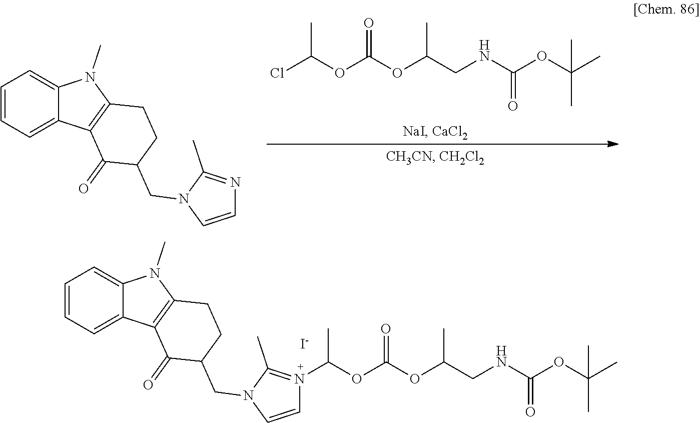

833 mg (2.84 mmol) of ondansetron, 851 mg (5.68 mmol) of sodium iodide, and 95 mg (0.85 mmol) of calcium chloride were added at room temperature to a mixed solution of dichloromethane and acetonitrile of 800 mg (2.84 mmol) of carbonic acid 1-chloroethyl 2-[[(1,1-dimethylethoxy)carbonyl]amino]-1-methylethyl ester and stirred at 45° C. overnight. The reaction solution was filtered, and then the reaction solution was condensed in a water bath set to 40° C. The residue was purified by silica gel column chromatography (10% methanol/chloroform) to obtain 239 mg (13%) of the title compound.

¹H-NMR (CDCl₃, δ): 1.26 (3/2H, d, J=7 Hz), 1.30-1.31 (3/2H, m), 1.38-1.47 (9H, m), 2.00 (3/2H, d, J=7 Hz), 2.05-2.09 (5/2H, m), 2.77-2.86 (1H, m), 3.09-3.13 (3H, m), 3.19-3.55 (5H, m), 3.68 (3/2H, s), 3.69 (3/2H, s), 4.42-4.94 (4H, m), 6.62-6.71 (1H, m), 7.24-7.43 (4H, m), 7.83-8.16 (2H, m)

### Reference Example 47

### 3-[1-[[(2-amino-1-methylethoxy)carbonyl]oxy]ethyl]-2-methyl-1-[(2,3,4,9-tetrahydro-9-methyl-4-oxo-1H-carbazol-3-yl)methyl]-1H-imidazolium chloride hydrochloride

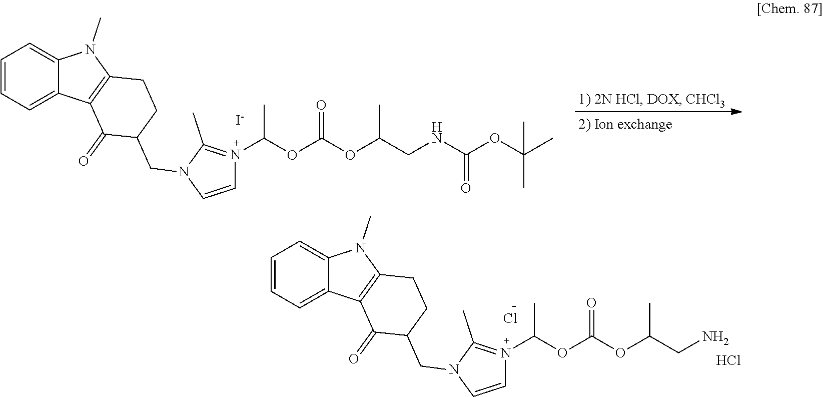

2 mL of 4 N hydrochloric acid/dioxane solution was added under cooling on ice to 2 mL of chloroform solution of 239 mg (0.36 mmol) of 3-[1-[[[2-[[(1,1-dimethylethoxy)carbonyl]amino]-1-methylethoxy]carbonyl]oxy]ethyl]-2-methyl-1-[(2,3,4,9-tetrahydro-9-methyl-4-oxo-1H-carbazol-3-yl)methyl]-1H-imidazolium iodide. The reaction solution was warmed to room temperature and left to stand still for 15 minutes. Thereafter, the solvent was distilled off under reduced pressure. The residue was dissolved in methanol and allowed to pass through Cl type ion exchange resin (DOWEX (registered trademark) 1×4 100-200 mesh), and the eluate was condensed under reduced pressure. Ethyl acetate was added to the residue and stirred for 1.5 hours. The precipitated crystals were collected by filtration to obtain 154 mg (84%) of the title compound.

¹H-NMR (DMSO-d6, δ): 1.24-1.31 (3H, m), 1.83 (3/2H, d, J=7 Hz), 1.84 (3/2H, d, 0.1=7 Hz), 1.96-2.01 (1H, m), 2.12-2.25 (1H, m), 2.82-2.87 (3H, m), 2.98-3.20 (5H, m), 3.75 (3/2H, s), 3.76 (3/2H, s), 4.33-4.41 (1H, m), 4.73-4.78 (1H, m), 4.90-4.96 (4H, m), 6.82-6.94 (1H, m), 7.21-7.29 (2H, m), 7.57-7.58 (1H, m), 7.88-7.91 (1H, m), 7.99-8.01 (1H, m), 8.07 (1/2H, d, J=2 Hz), 8.11 (1/2H, d, J=2 Hz), 8.42 (3H, br-s)

### Example 16

### [2-[[[1-(ondansetron)ethoxy]carbonyl]oxy]propyl]amino-chondroitin sulfate conjugate

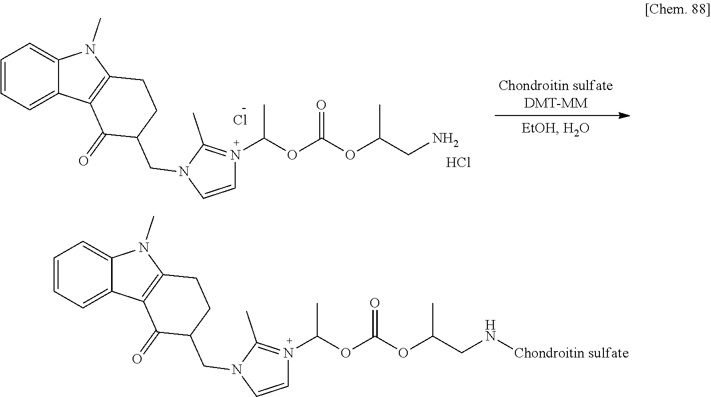

2 mL of ethanol was slowly added dropwise under stirring to 4.0 g (0.398 mmol) of 5% sodium chondroitin sulfate aqueous solution. To the mixed liquid, 1 mL of aqueous solution of 59 mg (0.11 mmol) of 3-[1-[[(2-amino-1-methylethoxy)carbonyl]oxy]ethyl]-2-methyl-1-[(2,3,4,9-tetrahydro-9-methyl-4-oxo-1H-carbazol-3-yl)methyl]-1H-imidazolium chloride hydrochloride was added, 1 mL of aqueous solution of 38 mg (0.136 mmol) of 4-(4,6-dimethoxy-1,3,5-triazin-2-yl)-4-methylmorpholinium chloride (DMT-MM) was then added, 1 mL of water and 5 mL of ethanol were further added, and the resultant liquid was stirred at room temperature overnight. 100 μL of 20% sodium chloride aqueous solution was added to the reaction solution, and ethanol was further added dropwise (5 mL) until immediately before the reaction solution became cloudy. The reaction solution was added dropwise under stirring to 16.5 mL of 93% ethanol, and 5 mL of ethanol was added to the mixed liquid and stirred for 1 hour, Precipitates were isolated using a centrifuge, washed twice with 90% ethanol, twice with ethanol, and twice with diethyl ether. The obtained precipitates were dried overnight using a vacuum pump to obtain 241 mg of the title compound. Based on values of integral in ¹H-NMR, the introduction ratio of ondansetron per whole disaccharide unit (glucurone acid) of chondroitin sulfate was 15 mol %.

### Reference Example 48

### N-[[[[[3-[[(1,1-dimethylethoxy)carbonyl]amino]-2-methylpropyl]amino]carbonyl]oxy]methyl]-N,N,α-trimethyl-10H-phenothiazin-10-ethanaminium chloride

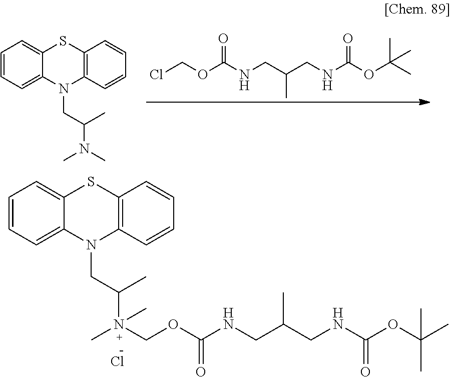

300 mg (L07 mmol) of promethazine was added to a dichloromethane solution of 304 mg (1.07 mmol) of N-[3-[[(1,1-dimethylethoxy)carbonyl]amino]-2-methylpropyl]carbamic acid chloromethyl ester, and the reaction solution was condensed at 70° C. over 1 hour and then stirred at the same temperature overnight. The residue was purified by silica gel column chromatography (4->20% methanol/chloroform) to obtain 396 mg (66%) of the title compound.

¹H-NMR (CDCl₃, δ): 0.86-0.88 (3H, m), 1.42 (9H, s), 1.62 (3H, d, 6 Hz), 1.87-1.88 (1H, m), 2.98-3.17 (4H, m), 3.33-3.43 (6H, m), 3.97-4.00 (1H, m), 4.27 (1H, dd, J=15, 6 Hz), 4.85-4.89 (1H, m), 5.16-5.18 (1H, m), 5.59-5.70 (2H, m), 7.00-7.29 (8H, m), 8.01 (1H, t, J=6 Hz)

### Reference Example 49

### N-[[[[(3-amino-2-methylpropyl)amino]carbonyl]oxy]methyl]-N,N,α-trimethyl-10H-phenothiazin-10-ethanaminium chloride hydrochloride

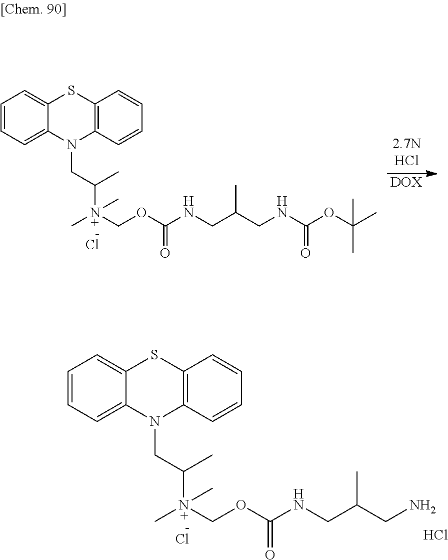

2 mL of 4 N hydrochloric acid/dioxane solution was added under cooling on ice to 2 mL of dioxane solution of 390 mg (0.69 mmol) of N-[[[[[3-[[(1,1-dimethylethoxy)carbonyl]amino]-2-methylpropyl]amino]carbonyl]oxy]methyl]-N,N,α-trimethyl-10H-phenothiazin-10-ethanaminium chloride. The reaction solution was warmed to room temperature and left to stand still for 1 hour. Thereafter, 2 mL of 4 N hydrochloric acid/dioxane solution was added again and left to stand still for 2 hours at room temperature, and then the solvent was distilled off under reduced pressure. Ethyl acetate was added to the residue and stirred for 1 hour. Crystals were collected by filtration to obtain 343 mg (99%) of the title compound.

¹H-NMR (DMSO-d6, δ): 0.90 (3H, d, J=7 Hz), 1.42 (3H, d, J=7 Hz), 1.89-1.97 (1H, m), 2.59-2.83 (2H, m), 2.96-3.16 (8H, m), 3.83-3.85 (1H, m), 4.14 (1H, d, J=15 Hz), 4.68 (1H, dd, J=15, 4 Hz), 5.35 (1H, d, J=9 Hz), 5.39 (1H, d, J=9 Hz), 7.05-7.08 (2H, m), 7.26-7.33 (6H, m), 8.13-8.17 (4H, m)

### Example 17

### [2-methyl-3-[[[(promethazine)methoxy]carbonyl]amino]propyl]amino-chondroitin sulfate conjugate

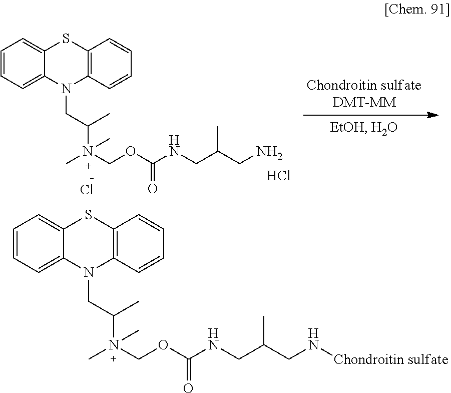

2 mL of ethanol was slowly added dropwise under stirring to 4.0 g (0.398 mmol) of 5% sodium chondroitin sulfate aqueous solution. To the mixed liquid, a solution of 40 mg (0.080 mmol) of N-[[[[(3-amino-2-methylpropyl)amino]carbonyl]oxy]methyl]-N,N,α-trimethyl-10H-phenothiazin-10-ethanaminium chloride hydrochloride in 1 mL of ethanol was added, a solution of 38 mg (0.136 mmol) of 4-(4,6-dimethoxy-1,3,5-triazin-2-yl)-4-methylmorpholinium chloride (DMT-MM) in 1 mL of ethanol was then added, 1 mL of ethanol and 1 mL of water were further added, and the resultant liquid was stirred at room temperature overnight. 100 μL of 20% sodium chloride aqueous solution was added to the reaction solution, and ethanol was further added dropwise (2.5 mL) until immediately before the reaction solution became cloudy. The reaction solution was added dropwise under stirring to 12 ML of 93% ethanol, and 4.5 mL of ethanol was added to the mixed liquid and stirred for 1 hour. Precipitates were isolated using a centrifuge, washed twice with 90% ethanol, twice with ethanol, and twice with diethyl ether. The obtained precipitates were dried overnight using a vacuum pump to obtain 196 mg of the title compound. Based on values of integral in ¹H-NMR, the introduction ratio of promethazine per whole disaccharide unit (glucuronic acid) of chondroitin sulfate was 18 mol %.

### Reference Example 50

### 1-[2-[(4-chlorophenyl)phenyl methoxy]ethyl]-1-[[[[[3-[[(1,1-dimethylethoxy)carbonyl]amino]-2-methylpropyl]amino]carbonyl]oxy]methyl]piperidinium chloride

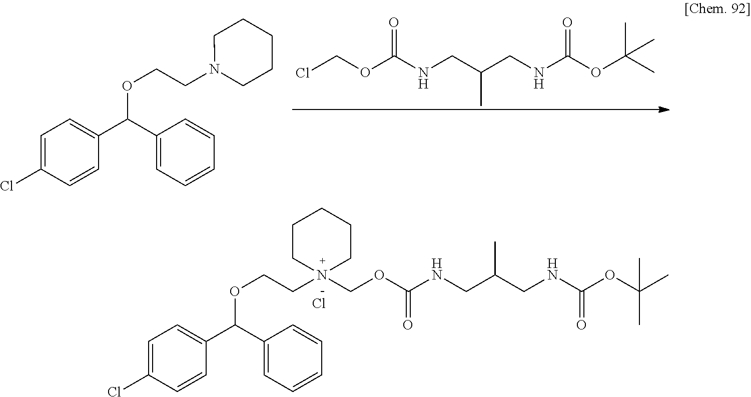

308 mg (0.93 mmol) of cloperastine was added at room temperature to a dichloromethane solution of 262 mg (0.93 mmol) of N-[3-[[(1,1-dimethylethoxy)carbonyl]amino]-2-methylpropyl]carbamic acid chloromethyl ester, and the reaction solution was condensed at 75° C. over 1 hour and then stirred at the same temperature overnight. The residue was purified by silica gel column chromatography (1→10% methanol/chloroform) to obtain 351 mg (62%) of the title compound.

¹H-NMR (CDCl₃, δ): 0.88 (3H, d, J=7 Hz), 1.42 (9H, s), 1.75-1.90 (7H, m), 2.97-3.18 (4H, m), 3.58-3.76 (4H, m), 3.90-3.92 (2H, m), 4.10-4.17 (2H, m), 5.23 (1H, br-s), 5.46-5.53 (3H, m), 7.25-7.35 (9H, m), 8.26 (1H, br-s)

### Reference Example 51

### 1-[[[[(3-amino-2-methylpropyl)amino]carbonyl]oxy]methyl]-1-[2-[(4-chlorophenyl)phenyl methoxy]ethyl]piperidinium chloride hydrochloride

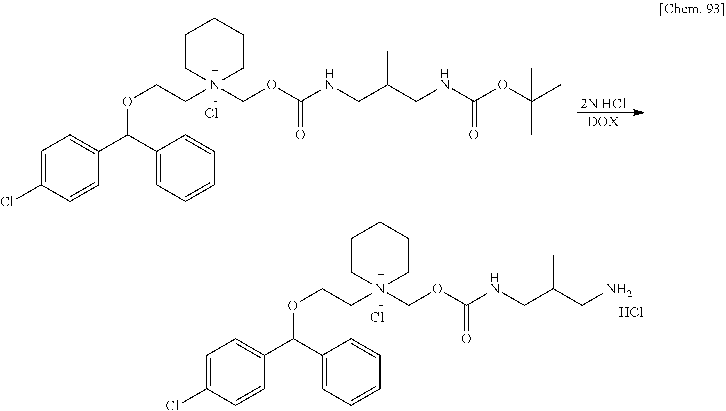

2 mL of 4 N hydrochloric acid/dioxane solution was added under cooling on ice to 2 mL of dioxane solution of 335 mg (0.56 mmol) of 1-[2-[(4-chlorophenyl)phenyl methoxy]ethyl]-1-[[[[[3-[[(1,1-dimethylethoxy)carbonyl]amino]-2-methylpropyl]amino]carbonyl]oxy]methyl]piperidinium chloride. The reaction solution was warmed to room temperature and left to stand still for 1 hour. Thereafter, the solvent was distilled off under reduced pressure. Ethyl acetate was added to the residue and stirred for 1 hour. Crystals were collected by filtration to obtain 231 mg (74%) of the title compound.

¹H-NMR (DMSO-d6, δ): 0.92 (3H, d, J=7 Hz), 1.52-1.60 (2H, m), 1.79-1.99 (5H, m), 2.58-2.61 (1H, m), 2.79-2.83 (1H, m), 2.98-3.08 (2H, m), 3.39-3.51 (4H, m), 3.75-3.79 (4H, m), 5.37-5.39 (2H, m), 5.69 (1H, br-s), 7.27-7.42 (9H, m), 8.22-8.26 (4H, m)

### Example 18

### [3-[[[(cloperastine)methoxy]carbonyl]amino]-2-methylpropyl]amino-chondroitin sulfate conjugate

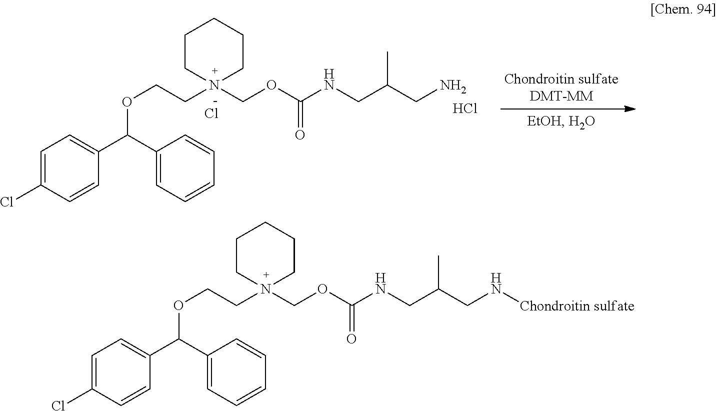

2 mL of ethanol was slowly added dropwise under stirring to 4.0 g (0.398 mmol) of 5% sodium chondroitin sulfate aqueous solution. To the mixed liquid, a solution of 43 mg (0.080 mmol) of 1-[[[[(3-amino-2-methylpropyl)amino]carbonyl]oxy]methyl]-1-[2-[(4-chlorophenyl)phenyl methoxy]ethyl]piperidinium chloride hydrochloride in 1 mL of ethanol was added, a solution of 38 mg (0.136 mmol) of 4-(4,6-dimethoxy-1,3,5-triazin-2-yl)-4-methylmorpholinium chloride (DMT-MM) in. 1 mL of ethanol was then added, 1 mL of ethanol and 1 mL of water were further added, and the resultant liquid was stirred at room temperature overnight, 100 μL of 20% sodium chloride aqueous solution was added to the reaction solution, and ethanol was further added dropwise (6 mL) until immediately before the reaction solution became cloudy. The reaction solution was added dropwise under stirring to 12 mL of 93% ethanol, and 1 mL of ethanol was added to the mixed liquid and stirred for 1 hour. Precipitates were isolated using a centrifuge, washed twice with 90% ethanol, twice with ethanol, and twice with diethyl ether. The obtained precipitates were dried overnight using a vacuum pump to obtain 215 mg of the title compound. Based on values of integral in ¹H-NMR, the introduction ratio of cloperastine per whole disaccharide unit (glucuronic acid) of chondroitin sulfate was 20 mol %.

### Reference Example 52

### 1-[[[[[3-[[(1,1-dimethylethoxy)carbonyl]amino]-2-methylpropyl]amino]carbonyl]oxy]methyl]-4-[[ethyl-hydroxy-1-oxo-2-phenylpropyl)amino]methyl]pyridinium chloride

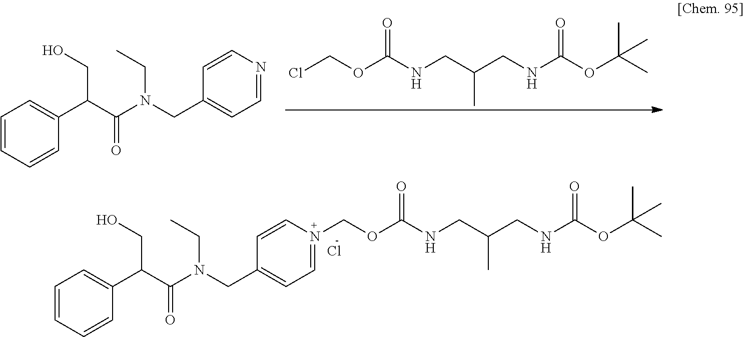

204 mg (0.72 mmol) of tropicamide was added at room temperature to a dichloromethane solution of 183 mg (0.65 mmol) of N-[3-[[(1,1-dimethylethoxy)carbonyl]amino]-2-methylpropyl]carbamic acid chloromethyl ester, and the reaction solution was condensed at 75° C. over 1 hour and then stirred at the same temperature overnight. The residue was purified by silica gel column chromatography (10→30% methanol/chloroform) to obtain 275 mg (75%) of the title compound.

¹H-NMR (CDCl₃, δ): 0.84 (3H, d, J=7 Hz), 1.04 (5/2H, t, J=7 Hz), 1.10 (1/2H, t, J=7 Hz), 1.41 (9H, s), 1.82-1.85 (1H, m), 2.97-3.10 (4H, m), 3.37-3.45 (1H, m), 3.69-3.82 (2H, m), 4.08-4.12 (1H, m), 4.39-4.42 (2H, m), 4.75-4.93 (1H, m), 5.17-5.21 (1H, m), 5.29-5.31 (1H, m), 6.55 (2H, s), 7.20-7.35 (5H, m), 7.55 (1H, br-s), 7.87 (1/3H, d, J=7 Hz), 8.04 (5/3H, d, J=7 Hz), 9.21 (2H, d, J=7 Hz)

### Reference Example 53

### 1-[[[[(3-amino-2-methylpropyl)amino]carbonyl]oxy]methyl]-4-[[ethyl(3-hydroxy-1-oxo-2-phenylpropyl)amino]methyl]pyridinium chloride hydrochloride

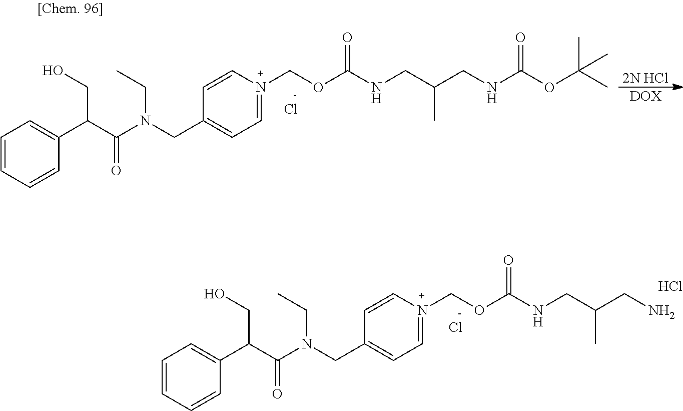

2 mL of 4 N hydrochloric acid/dioxane solution was added under cooling on ice to 2 mL of dioxane solution of 270 mg (0.48 mmol) of 1-[[[[[3-[[(1,1-dimethylethoxy)carbonyl]amino]-2-methylpropyl]amino]carbonyl]oxy]methyl]-4-[[ethyl(3-hydroxy-1-oxo-2-phenylpropyl)amino]methyl]pyridinium chloride. The reaction solution was warmed to room temperature and left to stand still for 1 hour. Thereafter, the solvent was distilled off under reduced pressure. Ethyl acetate was added to the residue and stirred for 1 hour. Crystals were collected by filtration to obtain 156 mg (65%) of the title compound.

¹H-NMR (DMSO-d6, δ): 0.88 (12/5H, d, J=7 Hz), 0.90 (3/5H, d, J=7 Hz), 1.02 (3H, t, J=7 Hz), 1.89-1.96 (1H, m), 2.54-3.03 (4H, m), 3.32-3.61 (3H, m), 3.89-4.05 (1H, m), 4.23-4.26 (1H, m), 4.71 (1H, d, J=18 Hz), 4.90 (1H, d, J=18 Hz), 4.96-5.04 (1H, m), 6.33 (2H, s), 7.18-7.38 (5H, m), 7.84 (2/5H, d, J=6 Hz), 7.89 (8/5H, d, J=6 Hz)f, 8.07-8.12 (4H, m), 9.00 (2/5H, d, J=6 Hz), 9.07 (8/5H, d, J=6 Hz).

### Example 19

### [2-methyl-3-[[[(tropicamide)methoxy]carbonyl]amino]propyl]amino-chondroitin sulfate conjugate

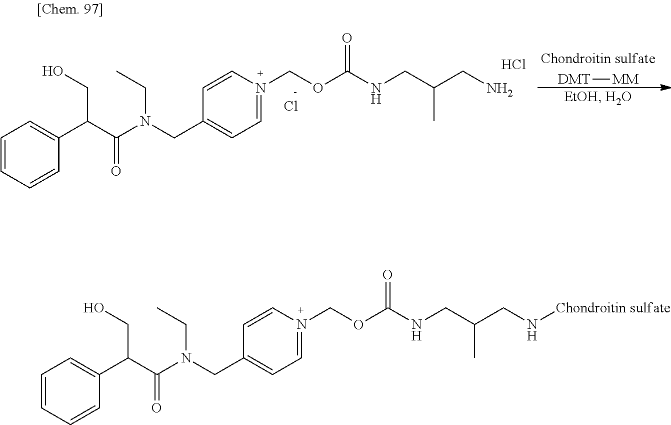

2 mL of ethanol was slowly added dropwise under stirring to 4.0 g (0.398 mmol) of 5% sodium chondroitin sulfate aqueous solution. To the mixed liquid, a solution of 40 mg (0.080 mmol) of 1-[[[[(3-amino-2-methylpropyl)amino]carbonyl]oxy]methyl]-4-[[ethyl(3-hydroxy-1-oxo-2-phenylpropyl)amino]methyl]pyridinium chloride hydrochloride in 1 mL of ethanol was added, a solution of 38 mg (0.136 mmol) of 4-(4,6-dimethoxy-1,3,5-triazin-2-yl)-4-methylmorpholinium chloride (DMT-MM) in 1 mL of ethanol was then added, 1 mL of ethanol and 1 mL of water were further added, and the resultant liquid was stirred at room temperature overnight. 100 μL of 20% sodium chloride aqueous solution was added to the reaction solution, and ethanol was further added dropwise mL) until immediately before the reaction solution became cloudy. The reaction solution was added dropwise under stirring to 12 mL of 93% ethanol, and 5 mL of ethanol was added to the mixed liquid and stirred for 1 hour. Precipitates were isolated using a centrifuge, washed twice with 90% ethanol, twice with ethanol, and twice with diethyl ether. The obtained precipitates were dried overnight using a vacuum pump to obtain 211 mg of the title compound. Based on values of integral in

¹H-NMR, the introduction ratio of tropicamide per whole disaccharide unit (glucuronic acid) of chondroitin sulfate was 18 mol %

### Reference Example 54

### N-[2-[[2-(methylthio)ethyl]amino]ethyl]carbamic acid 1,1-dimethylethyl ester

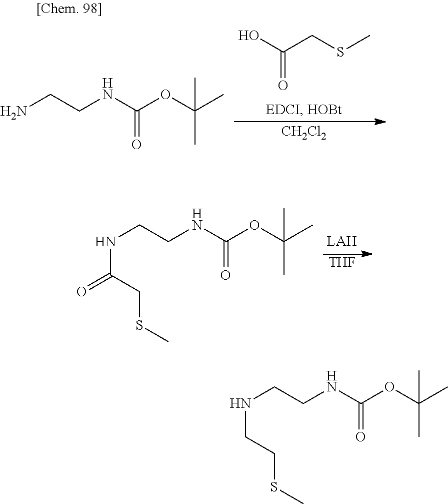

505 mg (3.74 mmol) of 1-hydroxybenzotriazole (HOBt) and 717 mg (3.74 mmol) of 1-ethyl-3-(3-dimethylaminopropyl)carbodiimide hydrochloride (EDCI) were added under cooling on ice to a dichloromethane solution of 500 mg (3.12 mmol) of N-[(1,1-dimethylethoxy)carbonyl]-1,2-ethylenediamine and 331 mg (3.12 mmol) of methylthioacetic acid, and the resultant liquid was warmed to room temperature and stirred overnight. The reaction solution was diluted with ethyl acetate and then washed with 10% potassium hydrogensulfate aqueous solution, saturated sodium bicarbonate water, and saturated saline. The organic layer was dried over anhydrous magnesium sulfate, and then the solvent was distilled off under reduced pressure to obtain 741 mg of N-[2-[[2-(methylthio)-1-oxoethyl]amino]ethyl]carbamic acid 1,1-dimethylethyl ester.

226 mg (5.95 mmol) of lithium aluminum hydride was added under cooling on ice to a tetrahydrofuran solution of 739 mg (2.98 mmol) of N-[2-[[2-(methylthio)-1-oxoethyl]amino]ethyl]carbamic acid 1,1-dimethylethyl ester thus obtained. The reaction solution was warmed to room temperature and stirred overnight, water and anhydrous magnesium sulfate were sequentially added to the reaction solution, and the resultant liquid was filtered through celite. The solvent was distilled off under reduced pressure, and then the residue was purified by silica gel column chromatography (chloroform/methanol) to obtain 278 mg (38%) of the title compound.

¹H-NMR (CDCl₃, δ): 1.45 (9H, s), 2.10 (3H, s), 2.65 (2H, t, J=7 Hz), 2.75 (2H, t, J=6 Hz), 2.82 (2H, t, J=7 Hz), 3.21-3.24 (2H, m), 4.94 (1H, br-s)

### Reference Example 55

### N-[2-[[(1,1-dimethylethoxy)carbonyl]amino]ethyl]-N-[2-(methylthio)ethyl]carbamic acid chloromethyl ester

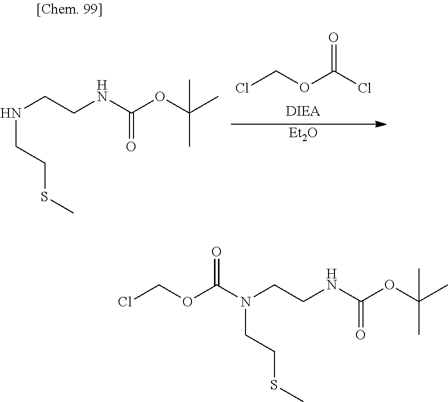

A diethyl ether solution of 179 mg (1.39 mmol) of chloromethyl chloroformate was added dropwise under cooling on ice to a diethyl ether solution of 273 mg (1.16 mmol) of N-[2-[[2-(methylthio)ethyl]amino]ethyl]carbamic acid 1,1-dimethylethyl ester and 226 mg (1.75 mmol) of N,N-diisopropylethylamine. The reaction solution was warmed to room temperature and stirred overnight, water was then added to the reaction solution, and the resultant liquid was extracted with diethyl ether. The obtained organic layer was washed with 10% potassium hydrogensulfate aqueous solution, saturated sodium bicarbonate water, and saturated saline and dried over anhydrous magnesium sulfate, and the solvent was distilled off under reduced pressure. The residue was purified by silica gel column chromatography (12%→50% ethyl acetate/hexane) to obtain 317 mg (84%) of the title compound.

¹H-NMR (CDCl₃, δ): 1.44 (9H, s), 2.14 (3/2H, s), 2.15 (3/2H, s), 2.66 (1H, t, J=7 Hz), 2.70 (1H, t, J=7 Hz), 3.28-3.53 (6H, m), 4.74 (1/2H, br-s), 4.84 (1/2H, br-s), 5.79 (2H, s)

### Reference Example 56

### 3-[[[[[2-[[(1,1-dimethylethoxy)carbonyl]amino]ethyl][2-(methylthio)ethyl]amino]carbonyl]oxy]methyl]-2-methyl-1-[(2,3,4,9-tetrahydro-9-methyl-4-oxo-1H-carbazol-3-yl)methyl]-1H-imidazolium chloride

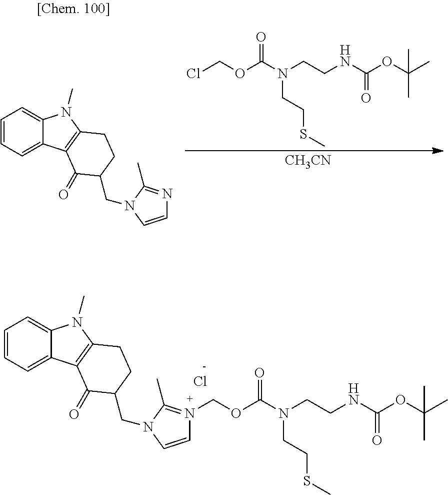

285 mg (0.97 mmol) of ondansetron was added at room temperature to an acetonitrile solution of 317 mg (0.97 mmol) of N-[2-[[(1,1-dimethylethoxy)carbonyl]amino]ethyl]-N-[2-(methylthio)ethyl]carbamic acid chloromethyl ester and stirred at 85° C. overnight. The reaction solution was condensed in a water bath set to 40° C. The residue was purified by silica gel column chromatography (10→20% methanol/chloroform) to obtain 444 mg (72%) of the title compound.

¹H-NMR (CDCl₃, δ): 1.40 (9/2H, s), 1.41 (9/2H, s), 2.03-2.08 (1H, m), 2.09 (3/2H, s), 2.10 (3/2H, s), 2.16-2.77 (3H, m), 3.03 (3/2H, s), 3.06 (3/2H, s), 3.11-3.51 (9H, m), 3.70 (3H, s), 4.56-4.59 (1H, m), 4.74 (1/2H, dd, J=15, 7 Hz), 4.77 (1/2H, dd, J=15, 7 Hz), 5.16 (1/2H, br-s), 5.43 (1/2H, br-s), 6.12 (1H, d, J=12 Hz), 6.17 (1H, d, J=12 Hz), 7.25-7.33 (3H, m), 7.46 (1/2H, d, J=2 Hz), 7.62 (1/2H, br-s), 7.85 (1H, br-s), 8.10 (1H, d, J=8 Hz)

### Reference Example 57

### 3-[[[[(2-aminoethyl)[2-(methylthio)ethyl]amino]carbonyl]oxy]methyl]-2-methyl-1-[(2,3,4,9-tetrahydro-9-methyl-4-oxo-1H-carbazol-3-yl)methyl]-1H-imidazolium chloride hydrochloride

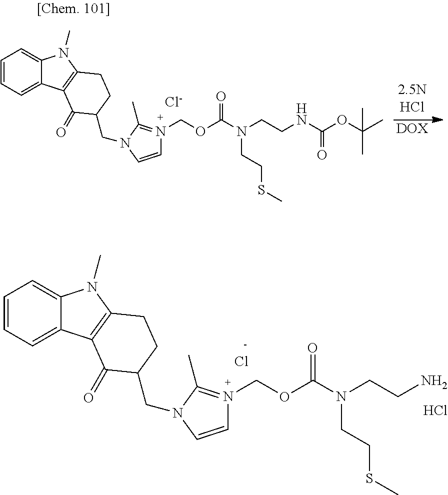

5 mL of 4 N hydrochloric acid/dioxane solution was added under cooling on ice to 3 mL of dioxane solution of 430 mg (0.68 mmol) of 3-[[[[[2-[[(1,1-dimethylethoxy)carbonyl]amino]ethyl][2-(methylthio)ethyl]amino]carbonyl]oxy]methyl]-2-methyl-1-[(2,3,4,9-tetrahydro-9-methyl-4-oxo-1H-carbazol-3-yl)methyl]-1H-imidazolium chloride. The reaction solution was warmed to room temperature and left to stand still for 2 hours. Thereafter, the solvent was distilled off under reduced pressure. Ethyl acetate was added to the residue and stirred and washed. Crystals were collected by filtration to obtain 396 mg (quantitative) of the title compound,

¹H-NMR (DMSO-d6, δ): 1.99 (3H, s), 2.06 (3/2H, s), 2.07 (3/2H, s), 2.16-2.20 (1H, m), 2.62-2.65 (2H, m), 2.80 (3/2H, s), 2.82 (3/2H, s), 2.95-3.05 (3H, m), 3.41-3.46 (2H, m), 3.49 (1H, t, J=7 Hz), 3.54 (1H, t, J=7 Hz), 3.75 (3H, s), 4.32-4.37 (1H, m), 4.72 (1H, dd, J=14, 7 Hz), 6.05-6.10 (2H, m), 7.22 (1H, t, J=8 Hz), 7.27 (1H, id, J=8, 2 Hz), 7.56 (1H, d, J=8 Hz), 7.76 (1/2H, d, J=3 Hz), 7.77 (1/2H, d, J=3 Hz). 7.80 (1/2H, d, J=3 Hz), 7.85 (1/2H, d, J=3 Hz), 7.99 (1H, d, J=8 Hz), 8.13 (3H, br-s)

### Example 20

### [2-[[2-(methylthio)ethyl][[(ondansetron)methoxy]carbonyl]amino]ethyl]amino-chondroitin sulfate conjugate

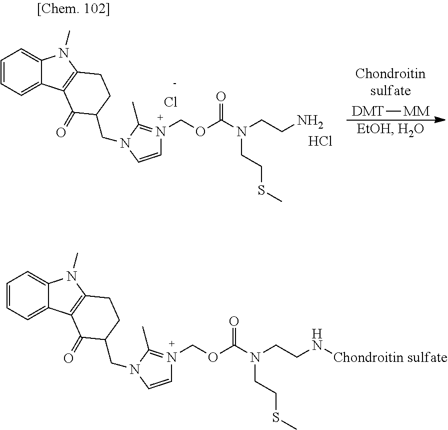

2 mL of ethanol was slowly added dropwise under stirring to 4.0 g (0.398 mmol) of 5% sodium chondroitin sulfate aqueous solution. To the mixed liquid, a solution of 46 mg (0.080 mmol) of 3-[[[[(2-aminoethyl)[2-(methylthio)ethyl]amino]carbonyl]oxy]methyl]-2-methyl-1-[(2,3,4,9-tetrahydro-9-methyl-4-oxo-1H-carbazol-3-yl)methyl]-1H-imidazolium chloride hydrochloride in 1 mL of ethanol was added, a solution of 38 mg (0.136 mmol) of 4-(4,6-dimethoxy-1,3,5-triazin-2-yl)-4-methylmorpholinium chloride (DMT-MM) in 1 mL of ethanol was then added, 1 mL of ethanol and 1 mL of water were further added, and the resultant liquid was stirred at room temperature overnight. 100 μL of 20% sodium chloride aqueous solution was added to the reaction solution, and ethanol was further added dropwise (2 mL) until immediately before the reaction solution became cloudy. The reaction solution was added dropwise under stirring to 12 mL of 93% ethanol, and 5 mL of ethanol was added to the mixed liquid and stirred for 1 hour. Precipitates were isolated using a centrifuge, washed twice with 90% ethanol, twice with ethanol, and twice with diethyl ether. The obtained precipitates were dried overnight using a vacuum pump to obtain 199 mg of the title compound. Based on values of integral in ¹H-NMR, the introduction ratio of ondansetron per whole disaccharide unit (glucuronic acid) of chondroitin sulfate was 25 mol %.

### Example 21

### [2-[[[(ondansetron)methoxy]carbonyl]amino]ethyl]amino-hyaluronic acid conjugate

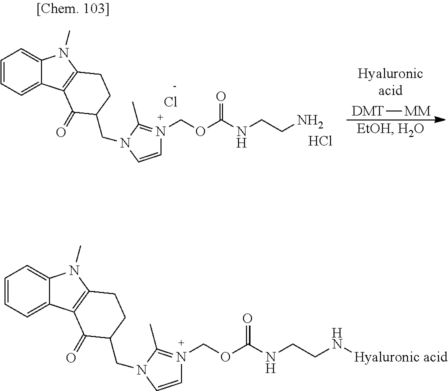

10 mL of ethanol was slowly added dropwise under stirring to 10 g (0.249 mmol) of 1% sodium hyaluronate aqueous solution. To the mixed liquid, a solution of 6.0 mg (0.0125 mmol) of 3-[[[[(2-aminoethyl)amino]carbonyl]oxy]methyl]-2-methyl-1-[(2,3,4,9-tetrahydro-9-methyl-4-oxo-1H-carbazol-3-yl)methyl]-1H-imidazolium chloride hydrochloride in 1 mL of ethanol was added, a solution of 5.9 mg (0.0212 mmol) of 4-(4,6-dimethoxy-1,3,5-triazin-2-yl)-4-methylmorpholinium chloride (DMT-MM) in 1 mL of ethanol was then added, 0.5 mL of ethanol and 2.5 mL of water were further added, and the resultant liquid was stirred at room temperature overnight. 1.5 mL of 20% sodium chloride aqueous solution and 30 mL of EtOH were added to the reaction solution to form precipitates and the supernatant of the suspension was removed. 10 mL of ethanol was further added thereto and stirred, and then the supernatant was removed. Thereafter, washed twice with 90% ethanol, twice with ethanol, and twice with diethyl ether. The obtained precipitates were dried overnight using a vacuum pump to obtain 70 mg of the title compound. Based on values of integral in ¹H-NMR, the introduction ratio of ondansetron per whole disaccharide unit (glucuronic acid) of hyaluronic acid was 5 mol %.

### Example 22

### [2-[[[(ondansetron)methoxy]carbonyl]amino]ethyl]amino-carboxymethyl cellulose conjugate

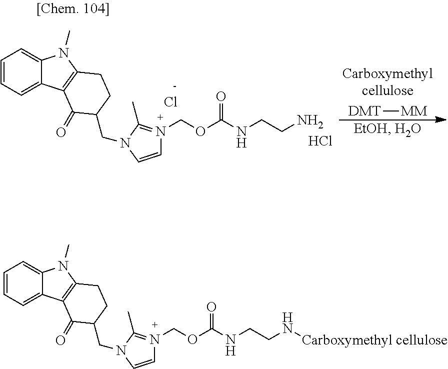

10 mL of ethanol was slowly added dropwise under stirring to 10 g (0.442 mmol) of 1% sodium carboxymethyl cellulose aqueous solution. To the mixed liquid, a solution of 10.7 mg (0.022 mmol) of 3-[[[[(2-aminoethyl)amino]carbonyl]oxy]methyl]-2-methyl-1-[(2,3,4,9-tetrahydro-9-methyl-4-oxo-1H-carbazol-3-yl)methyl]-1H-imidazolium chloride hydrochloride in 1 mL of ethanol was added, a solution of 10.3 mg (0.037 mmol) of 4-(4,6-dimethoxy-1,3,5-triazin-2-yl)-4-methylmorpholinium chloride (DMT-MM) in 1 mL of ethanol was then added, 0.5 mL of EtOH and 2.5 mL of water were further added, and the resultant liquid was stirred at room temperature overnight. 1 mL of 20% sodium chloride aqueous solution and 30 mL of EtOH were added to the reaction solution to form precipitates and the supernatant of the suspension was removed. 10 mL of ethanol was further added thereto and the supernatant was removed. Thereafter, washed twice with 90% ethanol, twice with ethanol, and twice with diethyl ether. The obtained precipitates were dried overnight using a vacuum pump to obtain 68 mg of the title compound. Based on the measurement result (247 nm) of a spectrophotometer, the introduction ratio of ondansetron per total weight of the polymer conjugate was 2 wt %:

### Example 23

### [4,4-dimethyl-3-[[[(ondansetron)methoxy]carbonyl]oxy]pentyl]amino-hyaluronic acid conjugate

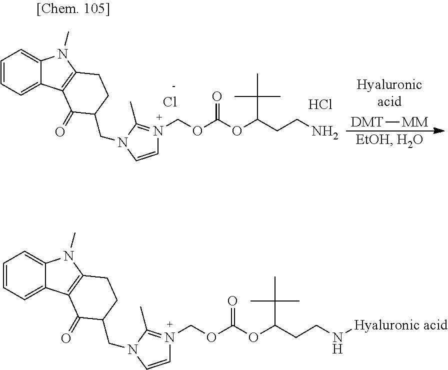

10 mL of ethanol was slowly added dropwise under stirring to 10 g (0.249 mmol) of 1% sodium hyaluronate aqueous solution. To the mixed liquid, 6.9 mg (0.0125 mmol) of 3-[[[[1-(2-aminoethyl)-2,2-dimethylpropoxy]carbonyl]oxy]methyl]-2-methyl-1-[(2,3,4,9-tetrahydro-9-methyl-4-oxo-1H-carbazol-3-yl)methyl]-1H-imidazolium chloride hydrochloride in 1 mL of ethanol was added, 5.9 mg (0.0213 mmol) of 4-(4,6-dimethoxy-1,3,5-triazin-2-yl)-4-methylmorpholinium chloride (DMT-MM) in 1 mL of ethanol was then added, 0.5 mL of ethanol and 2.5 mL of water were further added, and the resultant liquid was stirred at room temperature overnight. 1.5 mL of 20% sodium chloride aqueous solution and 30 mL of EtOH were added to the reaction solution to form precipitates and the supernatant of the suspension was removed. 10 mL of ethanol was further added thereto and stirred, and then the supernatant was removed. Thereafter, washed twice with 90% ethanol, twice with ethanol, and twice with diethyl ether. The obtained precipitates were dried overnight using a vacuum pump to obtain 65 mg of the title compound. Based on values of integral in ¹H-NMR, the introduction ratio of ondansetron per whole disaccharide unit (glucuronic acid) of hyaluronic acid was 1 mol %.

### Example 24

### [3-[[[(promethazine)methoxy]carbonyl]oxy]propyl]amino-carboxymethyl cellulose conjugate

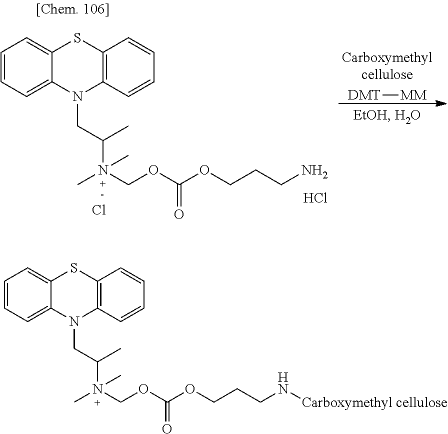

10 mL of ethanol was slowly added dropwise under stirring to 10 g (0.442 mmol) of 1% sodium carboxymethyl cellulose aqueous solution. A solution of 10.8 mg (0.022 mmol) of N-[[[(3-aminopropoxy)carbonyl]oxy]methyl]-N,N,α-trimethyl-10H-phenothiazin-10-ethanaminium chloride hydrochloride in 1 mL of ethanol was added, a solution of 10.3 mg (0.037 mmol) of 4-(4,6-dimethoxy-1,3,5-triazin-2-yl)-4-methylmorpholinium chloride (DMT-MM) in 1 mL of ethanol was then added, 0.5 mL of EtOH and 2.5 mL of water were further added, and the resultant liquid was stirred at room temperature overnight. 1 mL of 20% sodium chloride aqueous solution and 30 mL of EtOH were added to the reaction solution to form precipitates and the supernatant of the suspension was removed. 10 mL of ethanol was further added thereto and the supernatant was removed. Thereafter, washed twice with 90% ethanol, twice with ethanol, and twice with diethyl ether. The obtained precipitates were dried overnight using a vacuum pump to obtain 69 mg of the title compound. Based on the measurement result (249 nm) of a spectrophotometer, the introduction ratio of promethazine per total weight of the polymer conjugate was 0.7 wt %.

### Example 25

### [2-[[[(ondansetron)methoxy]carbonyl]amino]ethyl]amino-polyglutamic acid conjugate

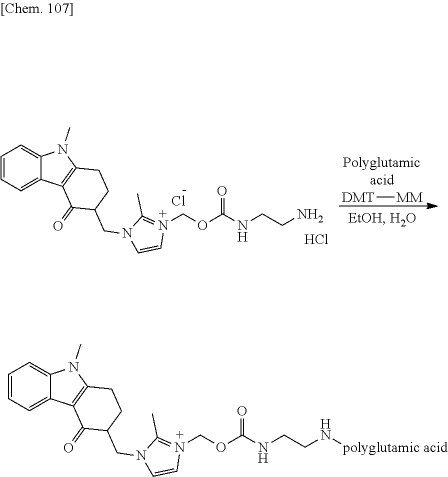

2 mL of ethanol was slowly added dropwise under stirring to 3.33 g (0.662 mmol) of 3% sodium polyglutamate aqueous solution. To the mixed liquid, a solution of 20.0 mg (0.033 mmol) of 3-[[[[(2-aminoethyl)amino]carbonyl]oxy]methyl]-2-methyl-1-[(2,3,4,9-tetrahydro-9-methyl-4-oxo-1H-carbazol-3-yl)methyl]-1H-imidazolium chloride hydrochloride in 1 mL of ethanol was added, a solution of 15.5 mg (0.056 mmol) of 4-(4,6-dimethoxy-1,3,5-triazin-2-yl)-4-methylmorpholinium chloride (DMT-MM) in 1 mL of ethanol was then added, 3 mL of ethanol and 3.7 mL of water were further added, and the resultant liquid was stirred at room temperature overnight. 100 μL of 20% sodium chloride aqueous solution was added to the reaction solution, and ethanol was further added dropwise (3 mL) until immediately before the reaction solution became cloudy. The reaction solution was added dropwise under stirring to 10 mL of 90% ethanol, and 5 mL of ethanol was added to the mixed liquid and stirred. Precipitates were isolated using a centrifuge, washed twice with 90% ethanol, twice with ethanol, and twice with diethyl ether. The obtained precipitates were dried overnight using a vacuum pump to obtain 84 mg of the title compound. Based on the measurement result (247 nm) of a spectrophotometer, the introduction ratio of ondansetron per total weight of the polymer conjugate was 6 wt %.

### Example 26

### [2-[[[(ondansetron)methoxy]carbonyl]amino]ethyl]amino-alginic acid conjugate

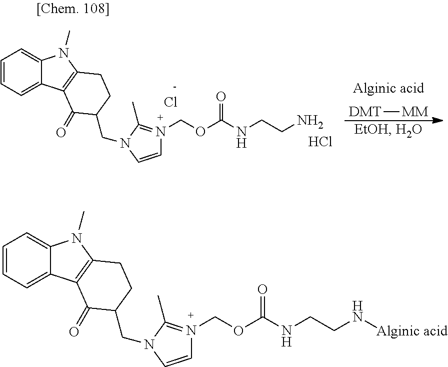

5 mL of water and 12 mL of ethanol were slowly added dropwise under stirring to 10 g (0.505 mmol) of 1% sodium alginate aqueous solution. To the mixed liquid, a solution of 12.2 mg (0.025 mmol) of 3-[[[[(2-aminoethyl)amino]carbonyl]oxy]methyl]-2-methyl-1-[(2,3,4,9-tetrahydro-9-methyl-4-oxo-1H-carbazol-3-yl)methyl]-1H-imidazolium chloride hydrochloride in 0.5 mL of ethanol was added, a solution of 11.8 mg (0.043 mmol) of 4-(4,6-dimethoxy-1,3,5-triazin-2-yl)-4-methylmorpholinium chloride (DMT-MM) in 0.5 mL of ethanol was then added, 2 mL of ethanol and 7.5 mL of water were further added, and the resultant liquid was stirred at room temperature overnight. 1 mL of 20% sodium chloride aqueous solution was added to the reaction solution and stirred. 200 mL of acetone was added to the reaction solution to form precipitates. After the supernatant was removed, washed 6 times with 90% acetone, twice with acetone, and twice with diethyl ether. The obtained precipitates were dried overnight using a vacuum pump to obtain 68 mg of the title compound. Based on the measurement result (247 nm) of a spectrophotometer, the introduction ratio of ondansetron per total weight of the polymer conjugate was 4 wt %.

### Example 27

### [2-[[[(ondansetron)methoxy]carbonyl]amino]ethyl]amino-polyacrylic acid conjugate

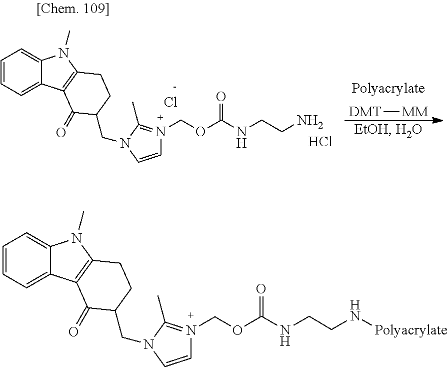

3 mL of ethanol was slowly added dropwise under stirring to 5 g (1.06 mmol) of 2% sodium polyacrylate aqueous solution. To the mixed liquid, a solution of 25.6 mg (0.053 mmol) of 3-[[[[(2-aminoethyl)amino]carbonyl]oxy]methyl]-2-methyl-1-[(2,3,4,9-tetrahydro-9-methyl-4-oxo-1H-carbazol-3-yl)methyl]-1H-imidazolium chloride hydrochloride in 1 mL of ethanol was added, a solution of 24.9 mg (0.090 mmol) of 4-(4,6-dimethoxy-1,3,5-triazin-2-yl)-4-methylmorpholinium chloride (DMT-MM) in 1 mL of ethanol was then added, 2 mL of water was further added, and the resultant liquid was stirred at room temperature overnight. The reaction solution was condensed, ethanol was distilled off, and then freeze dry was performed. The obtained solid substance was washed with 80% ethanol two times, with 90% ethanol two times, with ethanol two times, and further with diethyl ether two times. The obtained solid substance was dried overnight using a vacuum pump to obtain 74 mg of the title compound. Based on the measurement result (247 nm) of a spectrophotometer, the introduction ratio of ondansetron per total weight of the polymer conjugate was 8 wt %.

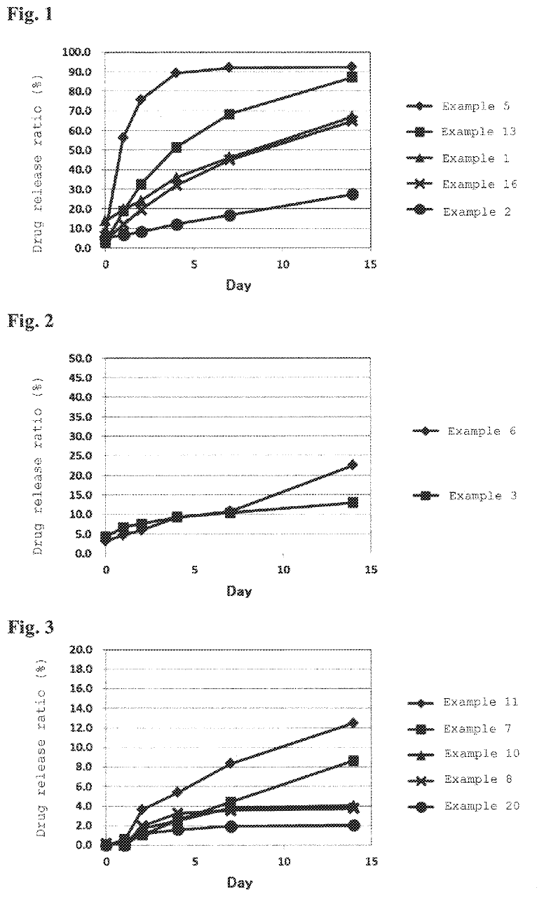

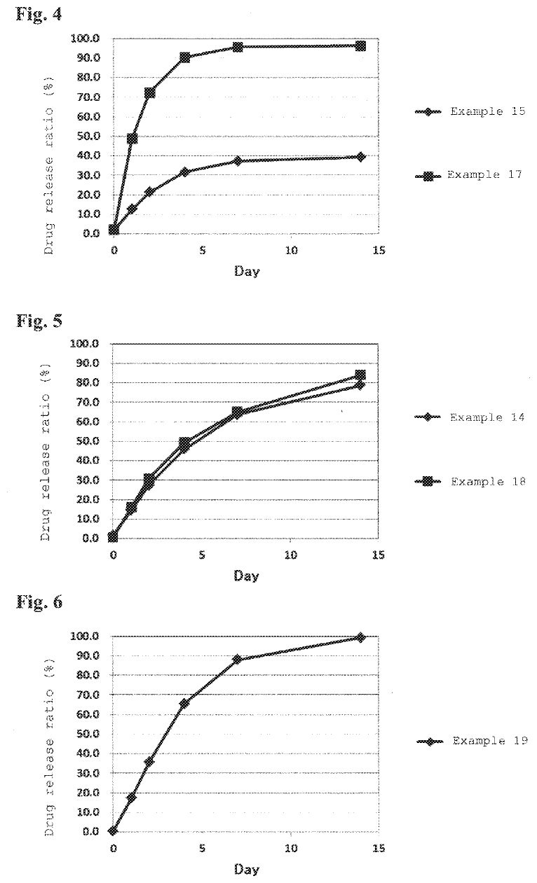

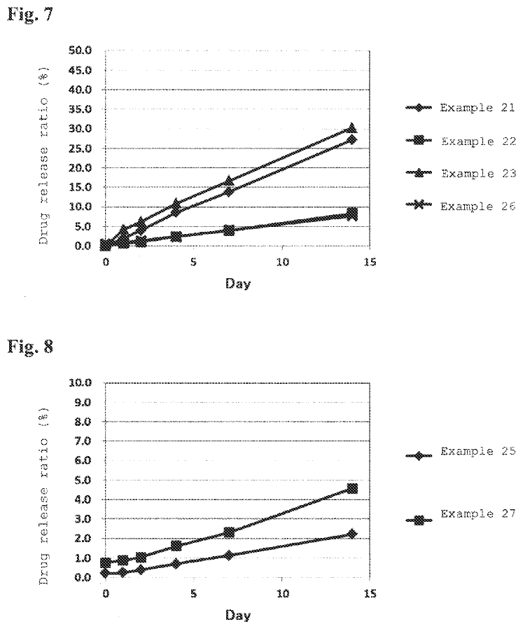

### Test Example 1 Drug Release Test of Drug-Polymer Conjugate

[Operation] Each evaluation polymer conjugate presented in Table 1 was dissolved in a concentration of 1.5 mg/mL in 20 mM of a sodium phosphate buffer solution having a pH of 7.0 and then dispensed. Immediately after dissolving, the drug-polymer conjugate amount and the release drug amount present in the solution were analyzed as an initial state (storage 0 day) by SEC-HPLC. Other dispensed injection liquid was stored under the condition of 36° C. immediately after dissolving, and the drug amount after each time elapsed was analyzed in the similar manner. From a ratio of the release drug amount and the drug-polymer conjugate amount at each time point which had been obtained in this way, a drug release ratio (%) was calculated. A relation between time and the drug release ratio is as shown in FIGS. 1 to 8.

The HPLC conditions are as follows.

Column: TSGgel α-3000 (7.8 mm×300 mm)

Flow rate: 0.5 mL/min

Temperature: 35° C.

Mobile phase: acetonitrile/physiological saline solution=1/2

TABLE 1

| Example No. | Compound name | Structure |
|---|---|---|
| Example 5 | [3-[[[(ondansetron)methoxy] carbonyl]oxy]-3-phenylpropyl] amino-chondroitin sulfate conjugate | 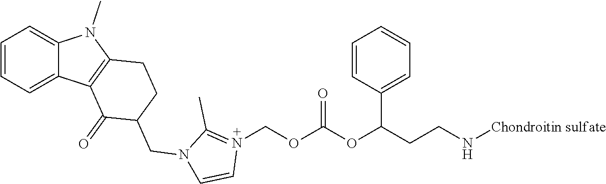 |
| Example 13 | [2-[[[(ondansetron)methoxy] carbonyl]oxy]ethyl ]amino-chondroitin sulfate conjugate | 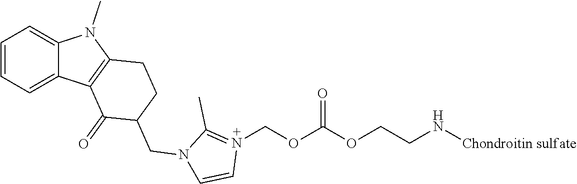 |
| Example 1 | [3-[[[(ondansetron)methoxy] carbonyl]oxy]propyl] amino-chondroitin sulfate conjugate | 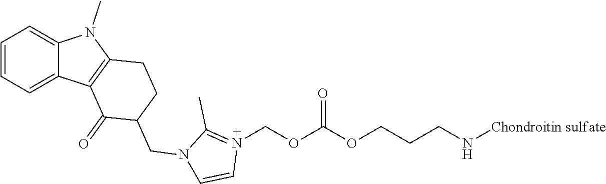 |
| Example 16 | [2-[[[1-(ondansetron)ethoxy] carbonyl]oxy]propyl] amino-chondroitin sulfate conjugate | 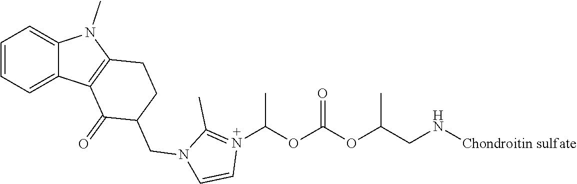 |
| Example 2 | [3-[[[(ondansetron)methoxy] carbonyl]oxy]butyl] amino-chondroitin sulfate conjugate | 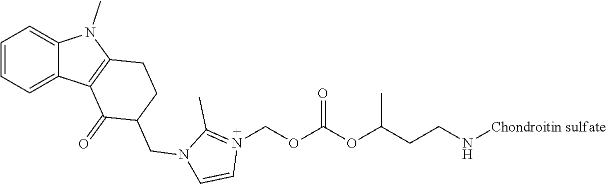 |
| Example 6 | [3-cyclohexyl-3- [[[(ondansetron)methoxy] carbonyl]oxy]propyl] amino-chondroitin sulfate conjugate | 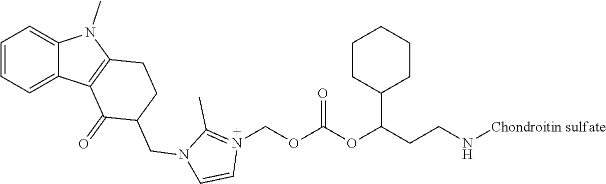 |
| Example 3 | [4,4-dimethyl-3- [[[(ondansetron)methoxy] carbonyl]oxy]pentyl] amino-chondroitin sulfate conjugate | 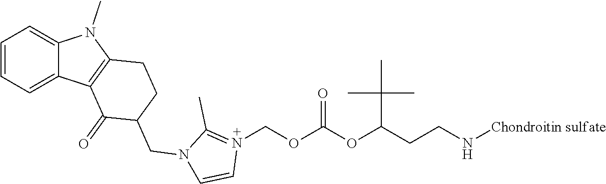 |
| Example 11 | [2-methyl-3- [[[(ondansetron)methoxay] carbonyl]oxy]propyl] amino-chondroitin sulfate conjugate | 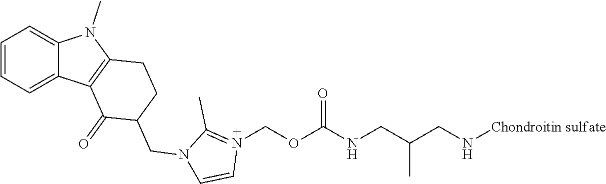 |
| Example 7 | [2- [[[(ondansetron)methoxy] carbonyl]amino]ethyl] amino-chondroitin sulfate conjugate | 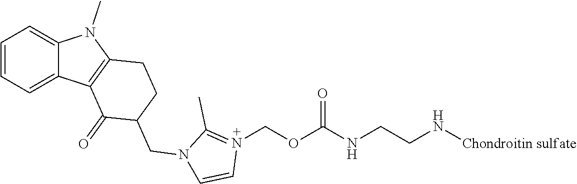 |
| Example 10 | [2-[methyl [[(ondansetron)methoxy] carbonyl]amino]ethyl] amino-chondroitin sulfate conjugate | 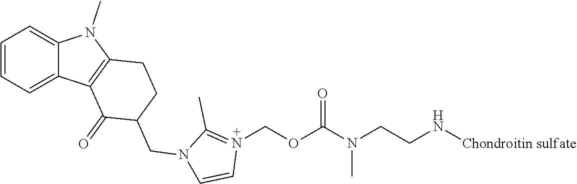 |
| Example 8 | [2-[[[2-methyl-1- (ondansetron)propoxy] carbonyl](1-methylethyl) amino]ethyl] amino-chondroitin sulfate conjugate | 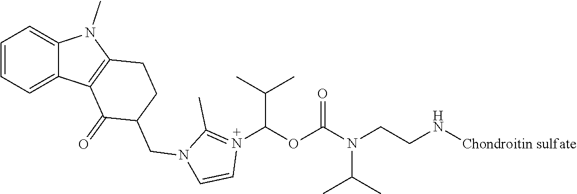 |
| Example 20 | [2-[[2-(methylthio) ethyl][[(ondansetron) methoxy]carbonyl]amino]ethyl] amino-chondroitin sulfate conjugate | 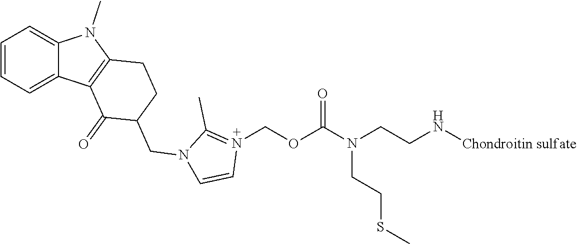 |
| Example 15 | [3-[[[(promethazine)methoxy] carbonyl]oxy]propyl] amino-chondroitin sulfate conjugate | 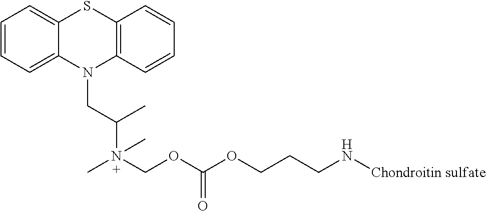 |
| Example 17 | [2-methyl-3- [[[(promethazine)methoxy] carbonyl]amino]propyl] amino-chondroitin sulfate conjugate | 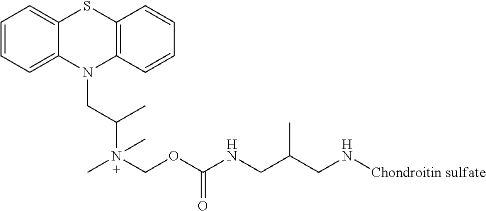 |
| Example 14 | [3-[[[(cloperastine)methoxy] carbonyl]oxy]propyl] amino-chondroitin sulfate conjugate | 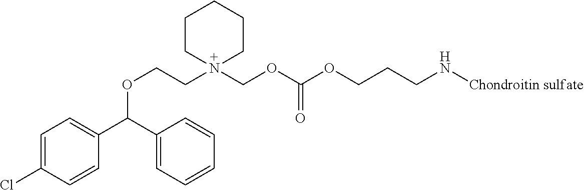 |
| Example 18 | [3-[[[(cloperastine)methoxy] carbonyl]amino]-2- methylpropyl]amino-chondroitin sulfate conjugate | 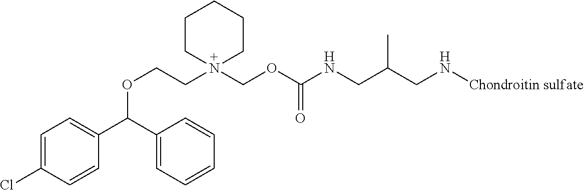 |
| Example 19 | [2-methyl-3- [[[(tropicamide)methoxy] carbonyl]amino]propyl] amino-chondroitin sulfate conjugate | 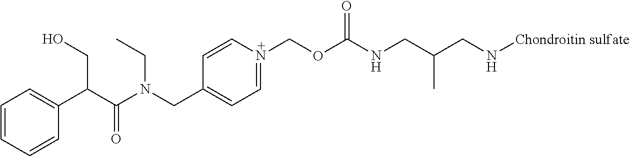 |
| Example 21 | [2-[[[(ondansetron)methoxy] carbonyl]amino]ethyl] amino-hyaluronic acid conjugate | 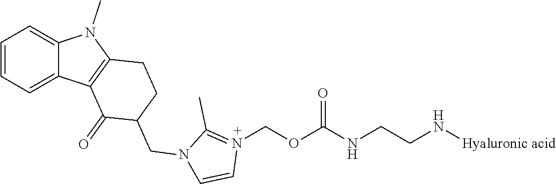 |
| Example 22 | [2-[[[(ondansetron)methoxy] carbonyl]amino]ethyl]amino- carboxymethyl cellulose conjugate | 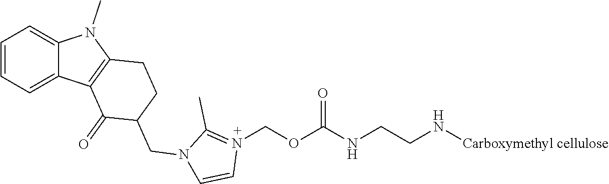 |
| Example 23 | [4,4-dimethyl-3- [[[(ondansetron) methoxy]carbonyl]oxy]pentyl] amino-hyaluronic acid conjugate | 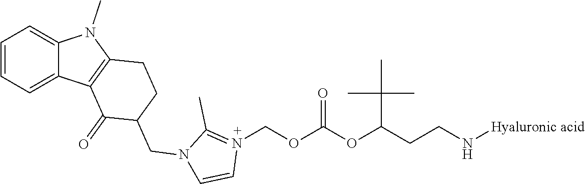 |
| Example 26 | [2-[[[(ondansetron) methoxy]carbonyl]amino]ethyl] amino-alginic acid conjugate | 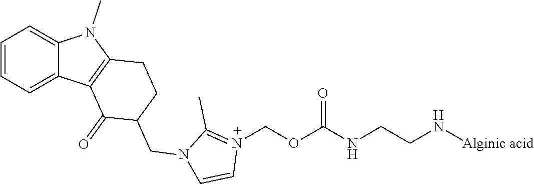 |
| Example 25 | [2-[[[(ondansetron) methoxy]carbonyl ]amino]ethyl] amino-polyglutamic acid conjugate | 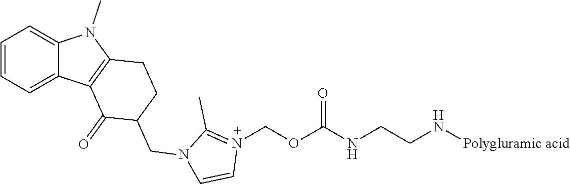 |
| Example 27 | [2-[[[(ondansetron) methoxy]carbonyl] amino]ethyl] amino-polyacrylic acid conjugate | 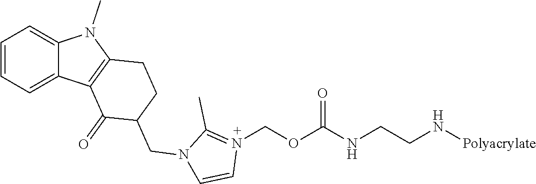 |

1. A compound represented by Formula (I) or a pharmaceutically acceptable salt thereof; 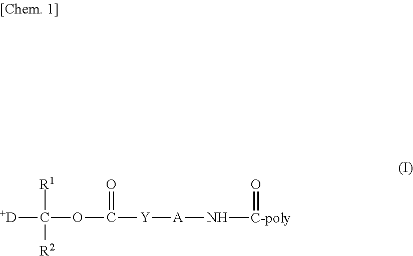
[in Formula (I), D⁺ is a structure forming a quaternary ammonium salt or an iminium salt from a tertiary amine compound or an imine compound D; a nitrogen atom of D⁺ forming the quaternary ammonium salt or the iminium salt and a carbon atom to which R¹ and R² bond are bonded to each other; Y is O or NR³; R¹, R², and R³ are each independently a hydrogen atom, a substituted or unsubstituted alkyl group, a substituted or unsubstituted cycloalkyl group, a substituted or unsubstituted alkenyl group, a substituted or unsubstituted cycloalkenyl group, a substituted or unsubstituted alkynyl group, a substituted or unsubstituted aromatic group, or a substituted or unsubstituted heterocyclic group; A is a substituted or unsubstituted bivalent hydrocarbon group, may contain one or more heteroatoms at a position except for both ends which are bonded to —Y— or —NH—, and the heteroatoms are each independently selected from the group consisting of —O—, —NH— which may have a substituent, and —S—; any two or three groups of R¹, R², R³, and A may combine together to form a ring; and Poly is a polymer residue and —C(═O)— adjacent to Poly is derived from a carboxy group of the polymer].

2. The compound according to claim 1 or a pharmaceutically acceptable salt thereof, wherein the Formula (I) is represented by the following Formula (II); 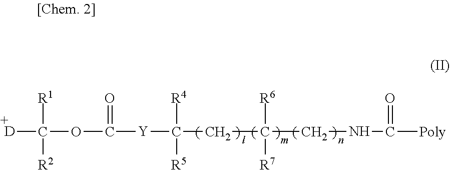
in Formula (II), D⁺, R¹, R², Y and Poly are as defined in claim 1; R⁴, R⁵, R⁶, and R⁷ are each independently a hydrogen atom, a substituted or unsubstituted alkyl group, a substituted or unsubstituted cycloalkyl group, a substituted or unsubstituted alkenyl group, a substituted or unsubstituted cycloalkenyl group, a substituted or unsubstituted alkynyl group, a substituted or unsubstituted aromatic group, or a substituted or unsubstituted heterocyclic group; any two or three groups of R¹, R², R³, R⁴, R⁵, R⁶, and R⁷ may combine together to form a ring; 1 and n are each independently 0, 1, or 2; and m is 0 or 1.

3. The compound according to claim 2 or a pharmaceutically acceptable salt thereof, wherein in Formula (I) or (II); R¹, R², R³, R⁴, R⁵, R⁶, and R⁷ are each independently a hydrogen atom; a substituted or unsubstituted linear or branched chain alkyl group having carbon number of 1 to 6; a substituted or unsubstituted cycloalkyl group having carbon number of 3 to 8; a substituted or unsubstituted linear or branched alkenyl group having carbon number of 2 to 6; a substituted or unsubstituted cycloalkenyl group having carbon number of 3 to 8; a substituted or unsubstituted linear or branched alkynyl group having carbon number of 2 to 6; a substituted or unsubstituted monocyclic or polycyclic aromatic group having carbon number of 6 to 14; or a substituted or unsubstituted 3- to 8-membered heterocyclic group containing at least one of a nitrogen atom, an oxygen atom, or a sulfur atom as a ring-constituting atom.

4. The compound according to claim 2 or a pharmaceutically acceptable salt thereof, wherein in Formula (I) or (II), a substituent of alkyl group, a substituent of cycloalkyl group, a substituent of alkenyl group, a substituent of cycloalkenyl group, a substituent of alkynyl group, a substituent of aromatic group, and a substituent of heterocyclic group in the groups represented by R¹, R², R³, R⁴, R⁵, and R⁷ are groups selected from a hydroxyl group, an alkyl group, a cycloalkyl group, an alkenyl group, a cycloalkenyl group, an alkynyl group, a halogen atom, an aromatic group, a heterocyclic group, an alkoxy group, a guanidino group, an alkylthio group, an alkoxycarbonyl group, an aryloxy group, an arylthio group, an acyl group, a substituted sulfonyl group, a heterocyclyloxy group, a heterocyclylthio group, an amide group, a ureido group, a carboxy group, a carbamoyl group, an oxo group, a thioxo group, a sulfamoyl group, a sulfo group, a cyano group, a nitro group, an acyloxy group, an azido group, a sulfonamide group, a mercapto group, an alkoxycarbonyl amino group, an aminocarbonyloxy group, a substituted sulfinyl group, a sulfamide group, an aminosulfonyloxy group, an alkoxysulfonyl amino group, a substituted sulfonyloxy group, an alkoxycarbonyl group, an alkoxycarbonyloxy group, an alkoxysulfonyl group, an imino group which may be substituted, an azo group which may be substituted, an Rx(Ry)N group, and an Rx(Ry)(Rz)N⁺ group, Rx, Ry, and Rz are each independently selected from the group consisting of a hydrogen atom, an alkyl group, a cycloalkyl group, an alkenyl group, a cycloalkenyl group, an alkynyl group, an aromatic hydrocarbon group, and a heterocyclic group, and at this time, two or more of Rx, Ry, and Rz may combine together to form a saturated or unsaturated hetero ring.

5. The compound according to claim 1 or a pharmaceutically acceptable salt thereof, wherein in Formula (I) or (II), Poly is a water-soluble polymer residue.

6. The compound according to claim 1, wherein in Formula (I) or (II), Poly is a polysaccharide residue.

7. The compound according to claim 1 or a pharmaceutically acceptable salt thereof, wherein in Formula (I) or (II), Poly is a glycosaminoglycan residue.

8. The compound according to claim 1 or a pharmaceutically acceptable salt thereof, wherein in Formula (I) or (II), Poly is a residue of chondroitin, chondroitin sulfate or hyaluronic acid.

9. The compound according to claim 1 or a pharmaceutically acceptable salt thereof, wherein a counter anion of D⁺ is an inorganic acid anion or an organic acid anion.

10. The compound according to claim 1 represented by the following Formula (XX) or a pharmaceutically acceptable salt thereof; 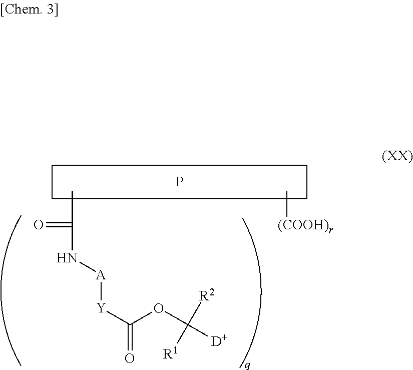
[in Formula (XX), D⁺, R¹, R², Y, and A are as defined in claim 1, and
a moiety represented by 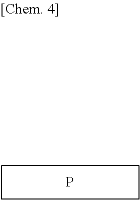
is a polymer excluding a group derived from a carboxy group and a carboxy group; q is the number of compounds condensed with the polymer; and r is the number of carboxy groups].

11. The compound according to claim 10 or a pharmaceutically acceptable salt thereof, wherein the compound represented by the above Formula (XX) is a compound represented by the following Formula (XXX): 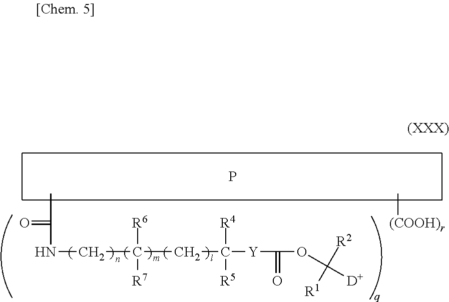
[in Formula (XXX), D⁺, R¹, and R² are as defined in claim 1; R⁴, R⁵, R⁶, and R⁷ are as defined in claim 2; and q and r are as defined in claim 10, and
a moiety represented by 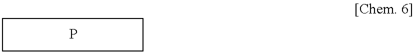
is a polymer excluding a group derived from a carboxy group and a carboxy group].

12. A method for producing a compound represented by the following Formula (I) or a pharmaceutically acceptable salt thereof, the method comprising a step of condensing a compound represented by the following Formula (III) and a polymer having a carboxy group represented by the following Formula (IV): 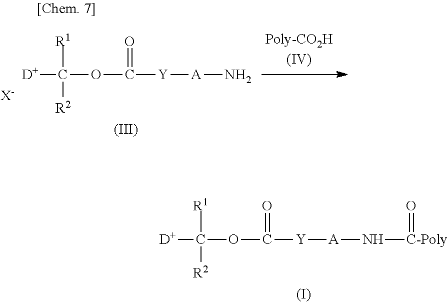
[in Formulae (I), (III), and (IV), D⁺, Y, A, R¹, R², and Poly are as defined in claim 1; X⁻ is a counter anion of D⁺, and the compound represented by Formula (III) may form a salt with an inorganic acid or an organic acid].

13. The production method according to claim 12, wherein the compound represented by the above Formula (III) is a compound represented by the following Formula (V), and the compound represented by the above Formula (I) is a compound represented by the following Formula (II): 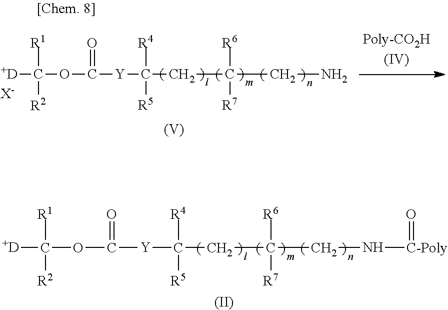
[in Formulae (II), (IV), and (V), D⁺, Y, R¹, R², and Poly are as defined in claim 1; R⁴, R⁵, R⁶, R⁷, l, n, and m are as defined in claim 2; X⁻ is a counter anion of D⁺, and the compound represented by Formula (V) may form a salt with an inorganic acid or an organic acid].

14. A method for producing a conjugate, the method comprising a step of bonding a tertiary amine compound containing a nitrogen atom capable of forming a quaternary ammonium salt or an imine compound capable of forming an iminium salt to a polymer having a carboxy group via a linker represented by the following Formula (VI): 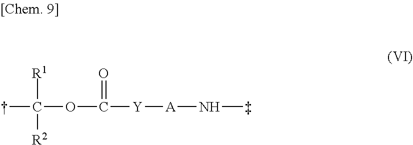
(wherein, R¹, R², Y, and A in the above (VI) are as defined in claim 1, symbol † is a node with the nitrogen atom forming the quaternary ammonium salt or the iminium salt, and symbol ‡ represents a node with carbonyl carbon derived from the carboxy group of the polymer).

15. The method for producing a conjugate according to claim 14, wherein the linker is represented by the following Formula (VII): 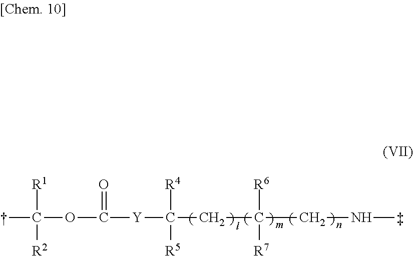
(wherein, Y, R¹, R², R⁴, R⁵, R⁶, R⁷, l, m, and n in the above (VII) are as defined in claim 2, symbol † is a node with the nitrogen atom forming the quaternary ammonium salt or the iminium salt, and symbol ‡ represents a node with carbonyl carbon derived from the carboxy group of the polymer).
